(19) United States
(12) Patent Application Publication
(10) Pub. No.: US 2010/0068150 A1
(43) Pub. Date: Mar. 18, 2010

(54) Selective Caspase Inhibitors

(75) Inventors: Matthew Bogyo, Redwood City, CA (US); Alicia B. Berger, Palo Alto, CA (US)

Correspondence Address: PETERS VERNY , L.L.P., 425 SHERMAN AVENUE, SUITE 230, PALO ALTO, CA, 94306, US

(73) Assignee: The Board of Trustees of the Leland Stanford Junior University, Palo Alto, CA (US)

(21) Appl. No.: 12/306,215

(22) PCT Filed: Jul. 6, 2007
(86) PCT No.: PCT/US07/15516

§ 371 Date: Sep. 30, 2009

Related U.S. Application Data
(60) Provisional application No. 60/819,233, filed on Jul. 7, 2006.

(51) Int. Cl. A61K 49/10; C07D 211/06; C07D 401/12; C07D 217/16; A61K 31/47; A61K 31/4436; A61K 31/4439; C12N 5/02; A61P 35/00

(52) U.S. Cl. 424/9.6; 546/226; 546/209; 546/208; 546/146; 514/307; 514/342; 514/354; 514/340; 435/325; 424/9.1

(57) ABSTRACT

Described here are novel, highly selective inhibitors and activity based probes (ABPs) for caspases 3, 7, 8, and 9 and legumain. The compounds selectively inhibit only certain caspases. A positional scanning combinatorial library (PSCL) approach was used to screen pools of peptide acyloxymethyl ketones (AOMKs) containing both natural and non-natural amino acids for activity against a number of purified recombinant caspases. These screens were used to identify structural elements at multiple positions on the peptide scaffold that could be modulated to control inhibitor specificity towards target caspases. Further disclosed are individual optimized covalent inhibitors that could also be equipped with various tags for use as activity based probes, as well as labeled substrates.

### CROSS-REFERENCE TO RELATED APPLICATIONS

This application claims priority from U.S. Provisional Patent Application No. 60/819,233 filed on Jul. 7, 2006, which is hereby incorporated by reference in its entirety.

### STATEMENT OF GOVERNMENTAL SUPPORT

This invention was made with U.S. Government support under NIH Grant No. R01 EB005011-01A1 and an NIH National Technology Center for Networks and Pathways grant U54 RR020843. The U.S. Government has certain rights in this invention.

### REFERENCE TO SEQUENCE LISTING, COMPUTER PROGRAM, OR COMPACT DISK

None

### BACKGROUND OF THE INVENTION

1. Field of the Invention

The present invention relates to the field of enzyme inhibition, more particularly to inhibition of cysteine proteases (caspases) with organic compounds, and in particular to specific inhibitors, probes and substrates which bind selectively to certain caspases.

2. Related Art

The clan CD cysteine proteases (known as caspases) plays a pivotal role in apoptosis, a tightly regulated form of programmed cell death essential for tissue homeostasis and elimination of damaged cells. Improper regulation of apoptosis is estimated to play a role in 70% of human diseases including cancer, certain neurodegenerative diseases, and reperfusion injury after ischemia (Reed, 1998). Thus tools to study caspases in both a basic and clinical setting are in high demand.

Caspases are present in the cytosol as inactive zymogens that become activated in response to specific death stimuli. Once activated, initiator caspases (caspase-8, 9, and 10) cleave and activate executioner caspases (caspases-3, 7). There are two primary pathways used to establish the cell death program. In general, the intrinsic pathway mediates response to cellular stress, such as DNA damage, and results in the activation of initiator caspase-9 while the extrinsic pathway is triggered by extracellular signals such as Fas binding to its cognate receptor, and leads to activation of initiator caspase-8. In both pathways initiator caspases cleave and activate downstream executioner caspases (Boatright et al., 2003; Denault and Salvesen, 2002; Salvesen, 2002; Thornberry and Lazebnik, 1998).

Since intrinsic and extrinsic apoptosis signals culminate in the activation of the same executioner caspases it has remained difficult to define the contribution of each pathway to apoptotic processes in vivo. Furthermore, activities of the executioner caspases increase over time causing them to dominate most non-specific caspase activity assays. This has prevented the detailed analysis of the kinetics of early activation events. In addition, surprisingly few tools are available for directly monitoring individual caspase activities in complex proteomes. Current strategies depend largely on antibody-based methods that can detect cleavage events of specific caspases. However, proteolytic cleavage is often not required for activation and a number of endogenous inhibitors exist that serve to control caspase activity through complex posttranslational mechanisms (Deveraux et al., 1999). Alternatively, caspase-targeted substrates and inhibitors can be used to directly monitor caspase activity. However, the value of virtually all commercial reagents is limited by their overall poor selectivity (James et al., 2004).

Past studies of substrate specificity of multiple caspase family members have focused on the use of positional scanning combinatorial libraries (PSCL) of fluorogenic peptide substrates (Backes et al., 2000; Thornberry et al., 1997). Our laboratory has previously used a similar positional scanning library approach to identify highly selective inhibitors of both recombinant and endogenously expressed proteases (Greenbaum et al., 2000; Greenbaum et al., 2002; Nazif and Bogyo, 2001).

### Selected Patents and Publications

Para et al., “Aspartate Ester Inhibitors of Interleukin-1β Converting Enzyme,” WO/98/16502 disclose compounds of the general formula

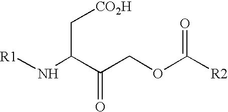

which can be seen to contain the AOMK functionality. R1 may be various substituted aryl and alkyl groups.

Keana et al., WO 99/18781, “Dipeptide Apoptosis Inhibitors and Use Thereof” discloses compounds of the general formula R1-AA-NH—CH(C—C—CO2R3)-C(O)—R2.

U.S. Pat. No. 6,531,474 to Wannamaker, et al., issued Mar. 11, 2003, entitled “Inhibitors of caspases,” discloses novel classes of compounds, which are caspase inhibitors, in particular interleukin-1β converting enzyme (“ICE”) inhibitors. These compounds are of the general formula

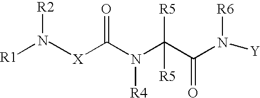

U.S. Pat. No. 6,689,84 to Bebbington, et al., issued Feb. 10, 2004, entitled “Carbamate caspase inhibitors and uses thereof,” discloses compounds of a general formula, which includes a ketone.

U.S. Pat. No. 6,800,619 to Charrier, et al., issued Oct. 5, 2004, entitled “Caspase inhibitors and uses thereof,” discloses compounds of a general formula, which also includes a ketone.

U.S. Pat. No. 6,878,743 to Choong, et al., issued Apr. 12, 2005, entitled “Small molecule inhibitors of caspases,” discloses compounds of a general formula having a ketone.

U.S. Pat. No. 6,566,338 Weber, et al., issued May 20, 2003, “Caspase inhibitors for the treatment and prevention of chemotherapy and radiation therapy induced cell death,” discloses compounds having a C-terminal aspartate-fluoro ketone (not fluoromethyl ketone) group, where “AA” is a residue of any natural or non-natural α amino acid or β amino acid or derivatives thereof.

U.S. Pat. No. 6,911,426 to Reed, et al., issued Jun. 28, 2005, entitled “Methods and compositions for derepression of IAP-inhibited caspase” discloses agents having various core peptide structures. The complete compounds have additional groups, e.g., [Boc-D-Lysine(2-Cl—Z)][Boc-D-Proline][1-adamantaneacetic acid].

Choe et al., “Substrate Profiling of Cysteine Proteases Using a Combinatorial Peptide Library Identifies Functionally Unique Specificities,” J. Biol. Chem., May 5, 2006, 281(18): 12824-12832, discloses a study of the substrate specificities of papain-like cysteine proteases (clan CA, family C1) papain, bromelain, and human cathepsins L, V, K, S, F, B, and five proteases of parasitic origin using a positional scanning synthetic combinatorial library. A bifunctional coumarin fluorophore was used that facilitated synthesis of the library and individual peptide substrates. Individual peptide substrates synthesized and tested for a quantitative determination of the specificity of the human cathepsins.

Kato et al., “Activity-based probes that target diverse cysteine protease families,” Nature Chemical Biology, 2005, 1, 33-38, discloses an Asp-AOMK probe which efficiently labeled caspase-3, caspase-6, caspase-7 and caspase-8 but not caspase-9, and probe bEVD-AOMK, which showed robust labeling of caspase-9 as well as caspase-3, caspase-7 and caspase-8. Also disclosed there is a solid phase synthetic method for the synthesis of cysteine protease inhibitors containing the acyloxymethyl ketone (AOMK) ‘warhead.’

Biotin-DEVD-AOMK is reported to be commercially available from (Merck Frosst Canada and Co.). See, Houde et al., “Caspase-7 Expanded Function and Intrinsic Expression Level Underlies Strain-Specific Brain Phenotype of Caspase-3-Null Mice,” The Journal of Neuroscience, Nov. 3, 2004, 24(44):9977-9984.

The caspase-1 and -3 inhibitors Ac-YVAD-aomk and DEVD-CHO re reported in Rami et al., “Okadaic acid-induced apoptosis in malignant glioma cells,” Neurosurg Focus, 2003, 14 (2):Article 4.

### BRIEF SUMMARY OF THE INVENTION

The following brief summary is not intended to include all features and aspects of the present invention, nor does it imply that the invention must include all features and aspects discussed in this summary.

Described below are the development and application of novel, highly selective inhibitors and activity based probes (ABPs) for caspases 3, 7, 8, and 9. A positional scanning combinatorial library (PSCL) was used to screen pools of peptide acyloxymethyl ketones (AOMKs) containing both natural and non-natural amino acids for activity against a number of purified recombinant caspases. These screens identified structural elements at multiple positions on the peptide scaffold that could be modulated to control inhibitor specificity towards target caspases. Using this screening data, we created individual optimized covalent inhibitors that could also be equipped with various tags for use as activity based probes (FIG. 1). We have developed several caspase-selective inhibitors and probes capable of specific inhibition and labeling of both recombinant and endogenous caspases. These reagents were applied to studies of the kinetics of caspase activation using a cell-free system in which intrinsic apoptosis could be activated by addition of cytochrome c and dATP. Using both general ABPs and specific inhibitors we have identified a full-length, uncleaved form of caspase-7 that becomes catalytically activated upon induction of apoptosome formation. Furthermore, the resulting inhibitors are irreversible and can also be converted to activity based probes by addition of small molecule tags such as biotin or fluorophores.

A caspase inhibitor according to the present invention may be represented by the following formula:

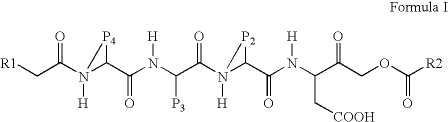

In some embodiments, P4 is omitted:

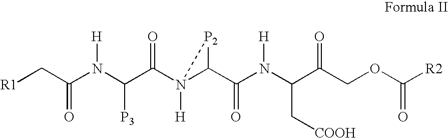

In the above formulas:

lines between P₂ and N and P₄ and N indicate bonds which exist only if P4 or P2 are Proline as set forth below;

R1 and R2 are independently H, NH2, aminocarbonyl, aryl, substituted aryl (including 2-nitro, 3-hydroxy), amino, aminocarbonyl, lower alkyl, cycloalkyl, or a label, and, referring to Formula I, P2, P3 and P4 are each a group independently selected from the possible P2, P3 and P4 groups listed in Table I:

TABLE I

| | Compound | | | |
|---|---|---|---|---|
| Primary Target | name | P4 | P3 | P2 |
| Caspase 3, 7, 8, 9 | AB28 | 6 | E | 8 |
| ″ | AB11 | D | E | P |
| Caspase 3, 7 | AB06 | D | 3 | V |
| ″ | AB13 | D | 34 | V |
| ″ | AB12 | D | 29 | V |
| Caspase 8 | AB20 | 29 | E | T |
| ″ | AB19 | 31 | E | 23 |
| | AB18 | 31 | E | T |
| Caspase 9 | — | L | E | H |
| ″ | AB38 | P | L | A |
| ″ | AB42 | I | F | P |
| ″ | AB41 | I | L | 38 |

Formula II is exemplified by compounds such as AB53, AB50, AB46, AB45, and AB37, that is, having P2 and P3, but not P4 positions. Table II is descriptive:

TABLE II

| | Compound | | |
|---|---|---|---|
| Primary Target | name | P3 | P2 |
| Caspase 3 | AB46 | E | 8 |
| ″ | AB50 | E | P |
| ″ | AB53 | 16 | P |

The above formulas may be represented by a single formula, i.e.,

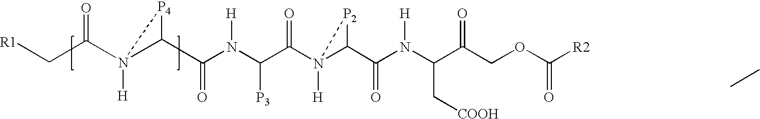

where the brackets indicate the optional inclusion of P4.

The above compounds may further comprise a label such as biotin or a fluorescent dye. This permits their use in methods where selected caspases are measured by their binding to the compound and the resulting signal, which may be detected, for example, by fluorescence within a test cell in which apoptotic activity, and increased induction of a selected caspase (e.g., caspase 3, 7, 8 or 9), is being studied. In a preferred embodiment, R1 is biotin. R1 labels may also include, e.g., fluorescein, rhodamine, digoxigenin or maleimide. R1 in unlabelled inhibitors may be, e.g., nitrophenol (NP), preferably 2-nitro, 3-hydroxy-benzyl, or amino

The above compounds are characterized by specifically selected amino acid side chains in P2, P3 and P4 positions, and, preferably, by an aspartate group in a P1 position, and irreversible binding moiety (“warhead”) comprising an AOMK group adjacent the P1 position.

In another aspect, the present invention comprises a selective fluorogenic substrate for specific caspase enzymes of the formula:

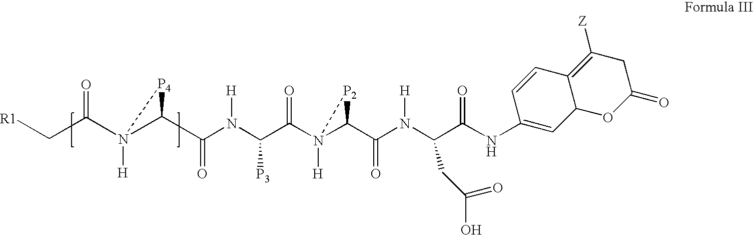

where:
dotted lines indicate bonds which exist only if P4 or P2 are proline as set forth below;
R1 is H, NH2, aminocarbonyl, aryl, substituted aryl (including 2-nitro, 3-hydroxy), amino, aminocarbonyl, lower alkyl, or cycloalkyl,
and P2, P3 and P4 are each a group independently selected from the possible P2, P3 and P4 groups listed in TABLE I, and
Z is selected from the group consisting of H, methyl and methyl acetamide [—CH2-C(═O)—NH2].

In this case, the substrate is not intended to inhibit the caspase activity. The AOMK group or other warhead is not present. The substrate may be used in conjunction with compounds or conditions intended to modulate caspase activity, and the resulting change in caspase activity (such as activity of a caspase inhibitor) can be measure by a change in fluorescence. The conjugate will normally emit light of a certain wavelength, but, upon proteolytic cleavage by the specific caspases, the free coumarin (e.g., 4-trifluoromethyl coumarin) emits a fluorescence at a different, longer wavelength that can be detected and is proportional to activity of the cognate caspase.

### BRIEF DESCRIPTION OF THE DRAWINGS

FIG. 1 shows development of caspase-specific inhibitors and active site probes. Solid phase chemistry was used to synthesize positional scanning combinatorial libraries (PSCLs) of nitrophenyl acetate (NP) capped peptide acyloxymethyl ketones (AOMKs). For all libraries, the P1 position directly adjacent to the reactive AOMK group was held constant as aspartic acid due to the strict cleavage requirements of caspases at this residue. One of the three remaining positions was also held constant (top to bottom, P2, P3 and P4, gray circles) as a single natural (a total of 19 excluding cysteine and methionine plus norleucine) or non-natural amino acid (from a set of 41 non-naturals see table below) while the other positions contained isokinetic mixtures of the natural amino acids (where positions are labeled “M” in circles). Single inhibitor compounds were selected after screening to determine the binding preference of individual caspases. Tags, such as biotin, were added in place of the nitrophenyl acetate cap of selective inhibitors to make activity based probes (ABPs);

FIGS. 2A through R are bar graphs, which show results of screening of PSCLs against recombinant caspases-3, 8 and 9. Purified recombinant caspases-3, 8 or 9 were pre-incubated with inhibitor sub-libraries followed by addition of fluorescent substrates. Fluorescence was measured at a set endpoint and residual enzyme activity was calculated from the ratio of normalized fluorescence signal of inhibited and control noninhibited samples (see Materials and Methods, below). Screening data for peptide libraries in which the constant position contains (FIG. 2A-I) natural amino acids and (FIG. 2J-R) non-natural amino acids as indicated along the horizontal axis. Cluster diagrams (also called heat maps) were also generated using a hierarchical clustering algorithm (Eisen et al., 1998) that converts residual activity values into a color format. For example, I in the P2 position shows 100% inhibition on the bar graph and, in a heat map (see Provisional priority case 60/819,233) shows 0% activity as a red or light gray color on a heat map.

FIGS. 3A and B shows analysis of inhibitor and probe selectivity by indirect competition and direct labeling of recombinant caspases. (a.) Indirect competition of a panel of inhibitors with the general caspase probe KMB01. Individual caspases-3, 7, 8 and 9 (100 nM each) were incubated with the indicated inhibitors for 30 minutes followed by a 30 minute incubation with KMB01. Samples were analyzed by SDS-PAGE and residual active site labeling was visualized by biotin blotting using streptavidin-HRP. (b.) Direct labeling of caspase active sites using specific ABPs. Equal amounts of active caspaseses-3, 8, and 9 (100 mM) were incubated together with increasing concentrations of each of the indicated biotinylated active site probes for 30 minutes. Active site labeling was visualized by SDS-PAGE analysis followed by biotin blotting using streptavidin HRP. (*) Indicates labeling of the full-length form of caspase-3 that is only observed using recombinant enzyme preparations.

FIGS. 4 A-D shows selective labeling of endogenous caspases in cell extracts and live cells with active site probes. (a.) Hypotonic 293 cytosolic extracts were induced to undergo intrinsic apoptosis by addition of cytochrome c/dATP. KMB01, bAB06 and bAB13 were added 10 minutes after activation and labeling of caspase active sites was carried out for three minutes. Samples were analyzed by SDS-PAGE followed by biotin blotting using streptavidin-HRP. (b) The identity of individual caspases was confirmed via immunoprecipitation using specific anti-sera for caspases-3, 7 and 9 (also see FIG. 5B for immunoprecipitation of caspases-3 and 7 after KMB01 labeling). Extracts (293) were activated with cytochrome c/dATP for 10 minutes, labeled by addition of indicated probes (100 nM final concentration for bAB06 and bAB13 and 10 μM final concentration for KMB01) and labeled caspases precipitated using specific anti-sera as described in the Methods section. I is input, P is pellet, S is supernatant after specific precipitation. (c.) Recombinant caspase-8 (100 nM) was either directly labeled or added to cell extracts (293) with or without cytochrome c/dATP activation and then labeled with the indicated probes (10 μM final concentration). The caspase-3 selective inhibitor AB06 (10 μM final concentration) was also added 10 minutes prior to probe addition to indicated samples. Labeling of caspases was monitored by SDS-PAGE followed by biotin blotting with streptavidin-HRP. (d.) Labeling of endogenous caspase-3 and 7 in intact Jurkat cells induced to undergo apoptosis through etoposide or anti-Fas treatment. Cells (3×10⁶) were incubated with apoptosis inducers for 15 hours and then labeled by incubation for an additional two hours with the panel of probes indicated. b-VAD-fmk, KMB01 and bAB19 were used at 10 μM concentration final. bAB06 and bAB13 were used at 1 μM final concentration. (*) denotes an endogenously biotinylated protein.

FIGS. 5 A-C show identification of novel caspase-7 activation intermediate in apoptotic cell extracts. (A) Cytosolic extracts (293) were induced to undergo intrinsic apoptosis by addition of cytochrome c and dATP for the indicated times. At the end of each time point, the general caspase probe KMB01 was added and extracts were incubated for an additional 30 minutes at 37° C. Labeled caspase active sites were visualized by SDS-PAGE analysis followed by blotting for biotin with streptavidin-HRP. The samples were analyzed by western blot using caspase-7 and 9 specific antibodies (lower panels). The identities of caspases are indicated based on immunoprecipitation experiments in (B). FL-C7 is full-length caspase-7, ΔN-C7 is full-length caspase-7 with the 23 N-terminal amino acids removed, p20 is mature large subunit of caspase-7 with N-terminal peptide removed, and p20+N-C7 is the mature large subunit of caspase-7 with the 23 residue N-peptide intact. P35-C9 is the predominant auto-processed mature form of caspase-9 large subunit, p33-C9 is an alternatively processed form of the mature large subunit of caspase-9. (b.) Immunoprecipitation of labeled caspases using specific anti-sera. Cytosolic extracts (293) were activated by addition of cytochrome c/dATP for 10 min (+cyt c/dATP) and then labeled for 30 min with the general caspase probe KMB01 or directly labeled with KMB01 without activation (-cyt c/dATP). Caspases were precipitated using specific anti-sera and analyzed by SDS-PAGE followed by blotting for biotin with streptavidin-HRP. I is input labeled extracts P is the immunoprecipitated pellet. (*) indicate cross reactive bands. (**) indicates forms of caspase-7 that are likely a result of the alternative transcription start site at methionine-45. (C) Inhibition of caspase activity by recombinant Bir3 domain. Cytosolic extracts were activated as in (A) for 5 minutes followed by addition of 1μM Bir3. KMB01 (20 μM) was added for 30 minutes to label residual caspase active sites as in (A).

FIGS. 6 A-E illustrate that full-length active caspase-7 has unique inhibitor specificity and is processed to mature forms by downstream executioner caspases. (A) The caspase-3 specific inhibitor causes accumulation of a catalytically active full-length form of caspase-7. Cytosolic extracts from 293 cells were activated with cytochrome c/dATP for the indicated times followed by labeling with KMB01 (left panel) and western blotting with a caspase-7 specific antibody (right panel) as in FIG. 5A. (B) Full-length caspase-7 does not accumulate in cells lacking active caspase-3. (B) The exact same experiment as (A) except extracts from MCF-7 cells were used in place of 293 extracts. (**) indicates forms of caspase-7 that are likely a result of the alternative transcription start site at methionine-45 (C) Quantification of the relative activity of FL-C7 in uninhibited 293 extracts (from FIG. 5A), AB06 treated 293 extracts (from (A)) and uninhibited MCF-7 extracts (from (B)) (D) Inhibitor specificity of full-length and processed forms of caspase-7. Extracts (293) were activated for 5 min with cytochrome c/dATP and then incubated with NP-capped AOMK inhibitors containing the indicated primary amino acid sequences for 5 minutes before KMB01 was added and allowed to label residual caspase active sites for 30 minutes. Samples were analyzed by SDS-PAGE followed by biotin blotting using strepavidin-HRP (left panel) or western blotting for caspase-7 (right panel). Identities of labeled caspases are indicated. (E) Specificity of AB06 after prolonged incubation times. Extracts were treated with AB06 at the indicated concentrations simultaneously with cytochrome c/dATP. At the indicated times after activation, samples were labeled with KMB01 (10 μM) for 30 minutes. Labeled caspases were resolved by SDS-PAGE analysis followed by biotin blotting using strepavidin-HRP.

FIGS. 7 A and B are cartoon representations of canonical executioner caspase activation (A) and a proposed model of caspase-7 activation via a “half-cleaved” intermediate; the peptide is removed by caspase-3 followed by cleavage of the linker region on both sides of the dimer by caspase-9 to produce the fully mature cleaved homodimer. In this model cleavage of the linker region is required to generate the catalytic active site (star); FIG. 7B shows an alternative model of caspase-7 activation in which initial processing of the uncleaved homodimer results in reorientation of the linker region and formation of a catalytically competent full-length caspase-7. This “half-cleaved” heterodimer is then a substrate for rapid processing by downstream executioner caspases-3, 6 or 7. Alternatively the N-peptide can be removed by caspase-3 followed by cleavage of the linker region to produce the “half-cleaved” complex. In both pathways a catalytically active full-length caspase-7 is produced (dashed box).

FIGS. 8 A-C show kinetics of caspase activation in 293 extracts treated with AB06 after activation of apoptosis. (a.) Extracts (293) were activated with cytochrome c/dATP as in FIGS. 5A and 6A and were treated with AB06 (10 μM final) ten minutes after activation. The general probe KMB01 was added for 30 at the indicated time points. Labeled caspases were analyzed by SDS-PAGE followed by blotting for biotin with streptavidin-HRP. The same samples were also analyzed for Caspase-7 and 9 protein levels by western blot using specific polyclonal antisera. (B) Caspase-9 immunoblots of the samples shown in FIG. 6A. (C) Caspase-9 immunoblots of the samples shown in FIG. 6A;

FIGS. 9 A-C is a schematic showing a synthetic scheme for compounds having P2, P3 and P4 positions;

FIGS. 10 A-F is a table showing structures of side chain compounds and their corresponding designations as “AB” numbers, as used in the present inhibitors, e.g., compound AB46 contains side chain “8” as shown in FIG. 10B;

FIG. 11 shows the structure of AB53 and AB53-Cy5;

FIG. 12A shows the structures of AB46, AB50 and Ab53; B shows gels of activity of these compounds in a RAW cell extract; C shows gels of activity against recombinant caspase-3;

FIG. 13 shows activity of AB46-Cy5, AB50-Cy5 and Ab53-CY5 against Raw cell extract (top three panels) and recombinant caspase (bottom three panels);

FIG. 14 shows gels from kidney (left, A) and spleen (right, B) labeled with AB46 and AB50 labeled with Cy-5;

FIG. 15A shows in vivo labeling of caspase-3 in the thymus using a scanner and in B, using blotting.

### DETAILED DESCRIPTION OF THE PREFERRED EMBODIMENT

### Overview

The detailed study of caspase activation during apoptosis requires sensitive tools that can be used to monitor proteases in a highly controlled and temporal fashion. While significant progress has been made towards understanding biochemical properties such as substrate specificity and active site topology of caspases, there remains a lack of effective small molecules to monitor specific caspase targets in the context of a complex proteome, intact cell, or whole organism. While several recent studies have made use of broad-spectrum activity based probes to monitor endogenous caspase activity in intact cells (Denault and Salvesen, 2003; Tu et al., 2006), the overall high reactivity of the probes prevented their use for real-time analysis of caspase activation. Described below are highly selective active site probes and inhibitors that could be used to dissect these specific activation events. Using a positional scanning approach with peptide acyloxymethyl ketones (AOMKs) containing both natural and non-natural amino acids we identified specificity elements that enabled the design of highly selective covalent inhibitors and active site probes. These compounds are likely to have great value for in vitro studies of caspase activation and have potential to be applied to in vivo imaging studies as has been recently reported for other classes of activity based probes (Blum et al., 2005; Joyce et al., 2004).

The reagents developed here were used to monitor caspases in cell free extracts upon activation of the intrinsic death pathway. While this system has been used extensively in the past, virtually all studies have relied on specific antibodies or exogenously added radiolabeled caspases to monitor the activation pathway. Thus it is likely that critical activation events that occur independently of proteolytic processing have been overlooked. We therefore chose to use a general probe to label all forms of active caspases at various time points after stimulation of the extracts with cytochrome c/dATP. These initial kinetic studies produced several interesting findings. Firstly, we found that the predominant active form of caspase-9 observed during activation of intrinsic apoptosis is likely the auto-catalytic p35 form that results from cleavage at the Asp 315 residue in the linker region. We also detected an active p33 form that results from alternate processing within the linker region. We did not detect the p37 form of caspase-9 that has been proposed to form through a caspase-3 mediated feedback loop (Slee et al., 1999). Furthermore, all forms of caspase-9 detected with the probes remained sensitive to inhibition by recombinant Bir3 domain suggesting that none of the caspase-9 forms observed represent a constitutively active feedback product.

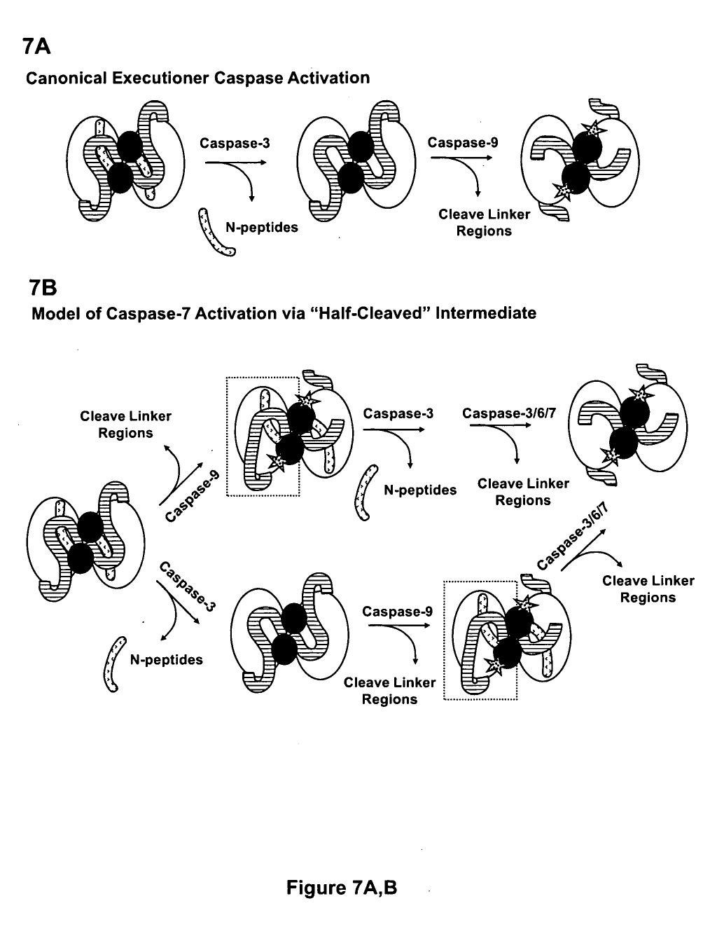

The second major finding that resulted from extract profiling studies was the appearance of a p37 full-length caspase-7 form that becomes catalytically activated in extracts upon stimulation of the intrinsic apoptosis pathway. The identification of an activated form of full-length caspase-7 without any change in total protein levels suggests that this activation results from a specific structural change in the uncleaved zymogen form. The notion that the full-length caspase-7 could become catalytically competent was surprising because executioner caspases are thought to initially form inactive homodimers that require cleavage between the large and small subunits by initiator caspases to become active (Boatright and Salvesen, 2003). In fact, high-resolution crystal structures of both zymogen and fully active mature forms of caspase-7 suggest that removal of the N-peptide and cleavage within the flexible linker region between the large and small subunits is required to orient key catalytic residues in the active site (Chai et al., 2001; Riedl et al., 2001). However, the crystal structures obtained for caspase-7 as the fully uncleaved, inactive homodimer and fully cleaved, active homodimer represent “snap shots” or local starting and endpoints in the activation process. Intermediates that could include a half-cleaved heterodimer are likely to exist but have yet to be characterized. Since the linker region where cleavage occurs is flexible, confirmation of one half of the dimeric complex could exert allosteric control over the other half resulting in its activation. Evidence for such a half cleaved caspase-7 heterodimer is supported by the work of Denault et al., In these studies various forms of caspase-7 that contain mutations that prevent either proteolytic activation or catalytic activity were used to generate half-cleaved heterodimers. These studies confirm an increase in probe labeling of the uncleaved half of the complex upon cleavage of the other half. Thus we believe that the increased labeling of the FL-C7 zymogen likely results from the partial processing of a homodimer by the apopto some leading to activation of the other half (FIG. 7). Since this activated form of full-length caspase-7 also shows distinct inhibitor binding properties, it is likely that it has a unique active site structure relative to the cleaved forms of caspase-7. This early intermediate may therefore have a specific functional role in the apoptosis pathway or it may represent a relatively transient intermediate that does not act upon substrates.

The present studies found accumulation of active FL-C7 (full length caspase 7) upon inhibition of the mature forms of caspase-3 and 7 using the newly developed selective inhibitor AB06. This is particularly interesting because active FL-C7 accumulates even at concentrations of inhibitor where the catalytic activity of caspase-9 is unaltered. Our data suggest that while initial activation of FL-C7, most likely in the form of a half-cleaved complex, is mediated by the apoptosome it cannot be efficiently processed by this complex to produce the fully cleaved homodimer. Furthermore, FL-C7 is processed with nearly normal kinetics in caspase-3 deficient MCF-7 cell extracts suggesting that processing of FL-C7 to its mature forms is not caspase-3 dependent. Taken together these results suggest that caspase-7 activation is a sequential process that involves the initial caspase-9 mediated half-cleavage of a homodimer complex that is then released and further processed primarily by downstream caspase-7 activity.

The development of highly selective inhibitors and active site probes provides a means to selectively monitor the role of each caspase during the process of apoptosis. Furthermore, the ability to monitor the temporal aspects of activation allows transient intermediates to be uncovered and their importance to be assessed.

The present invention comprises compounds, which are inhibitors of caspases selectively, e.g., inhibiting one (or at most four members [e.g., caspase 3, 7, 8, and 9] member of the human caspase family, or, in certain embodiments, legumain (asparaginyl endopeptidase) and no more than one caspase.

Table III below presents Kᵢ(app) values for select AB compounds. Kᵢ(app) values (also called Kₐₛₛ or K_{obs}/I) represent the speed of inhibitor binding to a target enzyme. Units are [M⁻¹s⁻¹]. NI indicates no inhibition at concentrations tested. Parent compounds AB09, ABo8, and AB07 that include the optimal substrate specificity sequences for caspases-3, 7, 8 and 9 respectively as determined by Thornberry and colleagues (Thornberry et al., 19970 are included between double lines.

TABLE III

| Target | | Specificity | Kᵢ(app) [M⁻¹s⁻¹] | | | |
|---|---|---|---|---|---|---|
| Caspase | Compound | Region | Caspase-3 | Caspase-7 | Caspase-8 | Caspase-9 |
| 3, 7, 8, 9 | ZVAD-fmk | V-A-D | 25922 | <5,000 | 203286 | <5,000 |
| | KMB01 | E-V-D | 577,913 | 288,213 | 164,052 | 175,210 |
| | AB11 | D-E-P-D | 2,482,333 | 199,341 | 580,547 | 47,362 |
| | AB28 | NH2-6-E-8-D | 1,020,213 | 272,619 | 817,077 | 300,767 |
| 3, 7 | AB09 | D-E-V-D | 10,922,261 | 1,529,040 | 1,077,839 | <5,000 |
| | AB06 | D-3-V-D | 7,456,511 | 968,070 | 32,909 | NI |
| | AB12 | D-29-V-D | 5,652,900 | 783,840 | 271,626 | NI |
| | AB13 | D-34-V-D | 3,416,050 | 279,519 | <5,000 | NI |
| | AB16 | 26-3-V-D | 484,495 | 24,185 | 121,650 | NI |
| | AB17 | 26-E-V-D | 781,733 | 448,155 | 126,323 | NI |
| 8 | AB08 | L-E-T-D | 127,835 | 19,424 | 599,788 | <5,000 |
| | AB20 | 29-E-T-D | 570,900 | 181,332 | 1,071,401 | 41,300 |
| | AB18 | 31-E-T-D | 216,040 | 234,945 | 572,012 | 12,320 |
| | AB19 | 31-E-23-D | 179,086 | 42,994 | 396,225 | NI |
| 9 | AB07 | L-E-H-D | 75,295 | 10,447 | 506,912 | 20,141 |
| | AB38 | P-L-A-D | 46,108 | 27,814 | 19,676 | 18,004 |
| | AB40 | I-L-A-D | 261,470 | 11,256 | 35,174 | 48,867 |
| | AB41 | I-L-38-D | 1,582,350 | 69,317 | 49,815 | 35,779 |
| | AB42 | I-F-P-D | 892,045 | 42,594 | 22,544 | 44,709 |

Finally, the data above in Table III are presented below in Table IV, which is organized by compound identifier, i.e., AB number.

TABLE IV

| Compound | | | | | | | | | | |
|---|---|---|---|---|---|---|---|---|---|---|
| | Specificity | | Caspase-3 | | Caspase-7 | | Caspase-8 | | Caspase-9 | |
| | Region | Target | Ki(app) | SD | Ki (app) | SD | Ki (app) | SD | Ki (app) | SD |
| ZVAD-fmk | V-A-D | 3, 7, 8, 9 | 25,922 | 143 | <5,000 | — | 203,286 | 8,499 | <5,000 | — |
| KMB01 | E-V-D | 3, 7, 8, 9 | 577,913 | 45,269 | 288,213 | 66,292 | 164,052 | 3,621 | 210 | 18,908 |
| AB06 | D-3-V-D | 3, 7 | 7,456,511 | 798,842 | 968,070 | 68,614 | 32,909 | 8,450 | — | |
| AB07 | L-E-H-D | 9 | 75,295 | 3,031 | 10,447 | 202 | 506,912 | 49,101 | 41 | 4,323 |
| AB08 | L-E-T-D | 8 | 127,835 | 14,476 | 19,424 | 537 | 599,788 | 92,747 | <5,000 | — |
| AB09 | D-E-V-D | 3, 7 | 10,922,261 | 1,698,557 | 1,529,040 | 77,901 | 1,077,839 | 122,447 | <5,000 | — |
| AB11 | D-E-P-D | 3, 7, 8, 9 | 2,482,333 | 105,882 | 199,341 | 28,649 | 580,547 | 20,679 | 47,362 | 3,041 |
| AB12 | D-29-V-D | 3, 7 | 5,652,900 | 239,568 | 783,840 | 33,955 | 271,626 | 1,403 | NI | — |
| AB13 | D-34-V-D | 3, 7 | 3,416,050 | 659,375 | 279,519 | 19,810 | <5,000 | — | NI | — |
| AB15 | 26-34-V-D | 3, 7 | 133,705 | 15,167 | ND | — | NI | — | NI | — |
| AB16 | 26-3-V-D | 3, 7 | 484,495 | 25,590 | 24,185 | 1,904 | 121,650 | 28,335 | NI | — |
| AB17 | 26-E-V-D | 3, 7 | 781,733 | 110,714 | 448,155 | 27,317 | 126,323 | 21,861 | NI | — |
| AB18 | 31-E-T-D | 8 | 216,040 | 3,111 | 234,945 | 30,823 | 572,012 | 158,775 | 12,320 | 3,986 |
| AB19 | 31-E-23-D | 8 | 179,086 | 8,237 | 42,994 | 1,325 | 396,225 | 92,743 | NI | — |
| AB20 | 29-E-T-D | 8 | 570,900 | 60,825 | 181,332 | 11,206 | 1,071,401 | 340,849 | 41,300 | 1,038 |
| AB28 | 6-E-8-D | 3, 7, 8, 9 | 1,020,213 | 293,979 | 272,619 | 34,569 | 817,077 | 99,766 | 300,767 | 26,860 |
| AB29 | D-E-11-D | None | 341,429 | ND | ND | — | 90,503 | 17,462 | NI | — |
| AB30 | D-30-11-D | None | 448,179 | 138,195 | ND | — | NI | — | NI | — |
| AB31 | D-30-V-D | None | 64,873 | 12,220 | ND | — | NI | — | NI | — |
| AB38 | P-L-A-D | 9 | 46,108 | 6,799 | 27,814 | 2,163 | 19,676 | 2,973 | 18,004 | 2,820 |
| AB40 | I-L-A-D | 9 | 261,470 | 30,600 | 11,256 | 903 | 35,174 | 2,790 | 48,867 | 5,435 |
| AB41 | I-L-38-D | 9 | 1,582,350 | 84,782 | 69,317 | 10,329 | 49,815 | 11,370 | 35,779 | 2,544 |
| AB42 | I-F-P-D | 9 | 892,045 | 544 | 42,594 | 6,225 | 22,544 | 2,482 | 44,709 | 2,465 |
| bAB06 | D-3-V-D | 3, 7 | 2,528,900 | 336,017 | 412,413 | 55,719 | 29,124 | 6,460 | NI | — |
| bAB13 | D-34-V-D | 3, 7 | 6,829,900 | 365,574 | 456,884 | 40,740 | <1.000 | | NI | — |
| bAB19 | 31-E-23-D | 8 | 192,225 | 87,193 | 40,011 | 5,210 | 152,956 | 32,590 | NI | — |
| bAB38 | P-L-A-D | 9 | 28,809 | 2,347 | 18,685 | 6,274 | 24,000 | 291 | 39,872 | 396 |

The following Table V exemplifies compounds described further below by AB number and structure:

TABLE V

| Com- | | | |
|---|---|---|---|
| pound | | | Mol |
| Name | Linker | Strucutre | Wt. |
| AB02 | Np-L-E- 36-D-DMBA | 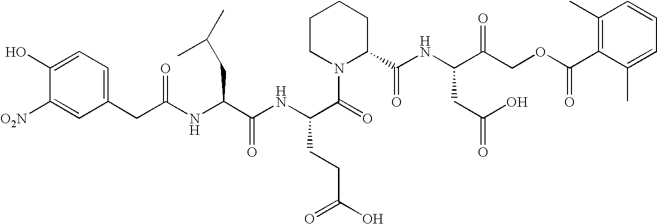 | 811.8 |
| AB03 | Np-28-16- 26-D-DMBA | 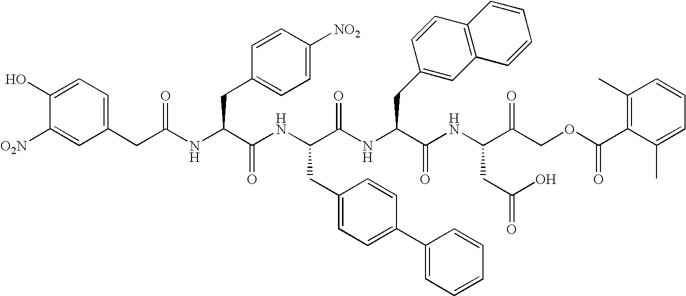 | 1072 |
| AB04 | Np-L-E- 16-D-DMBA | 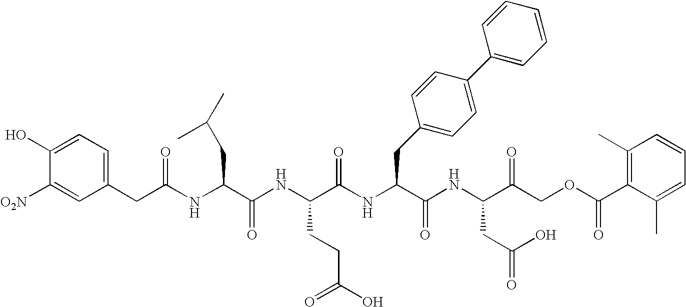 | 924 |
| AB05 | Np-38-3- 5-D-DMBA | 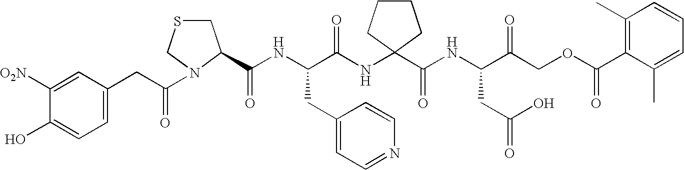 | 832.9 |
| AB06 | Np-D-3- V-D-DMBA | 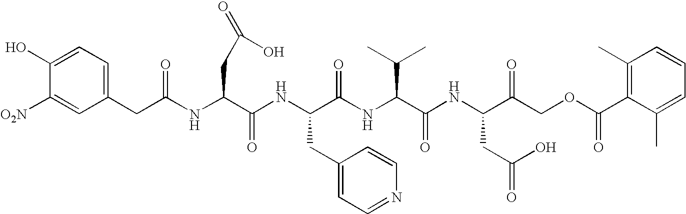 | 820.8 |
| AB07 | Np-L-E- H-D-DMBA | 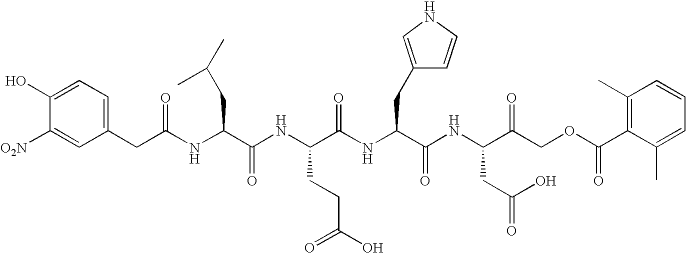 | 836.8 |
| AB08 | Np-L-E- T-D-DMBA | 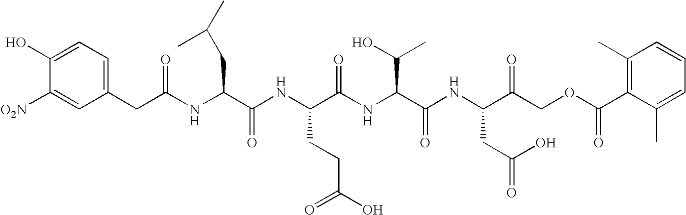 | 801.8 |
| AB09 | Np-D-E- V-D-DMBA | 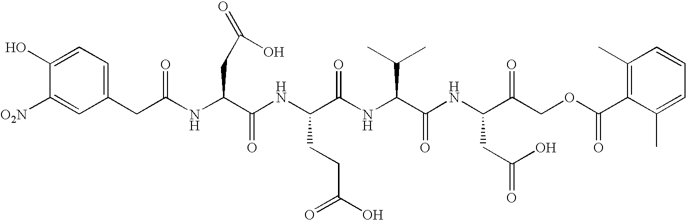 | 801.8 |
| AB10 | Np-27-31- 35-D-DMBA | 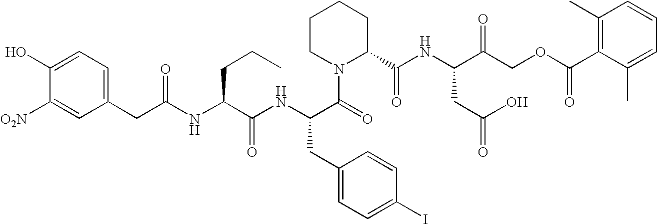 | 941.8 |
| AB11 | NP-D-E- P-D-DMBA | 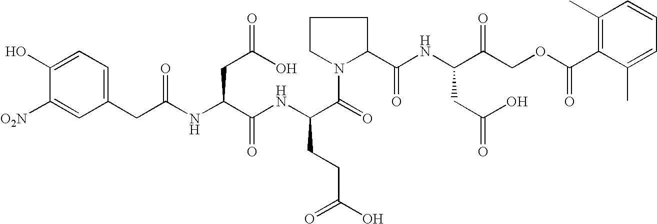 | 799.7 |
| AB12 | Np-D-E- 29-V-D-DMBA | 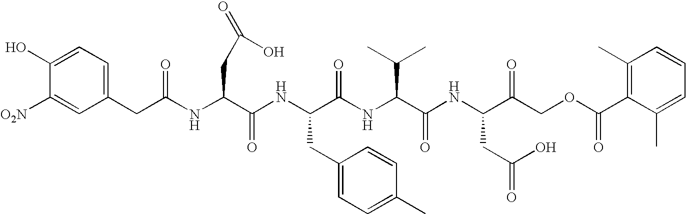 | 833.8 |
| AB13 | Np-D-E- 34-V-D-DMBA | 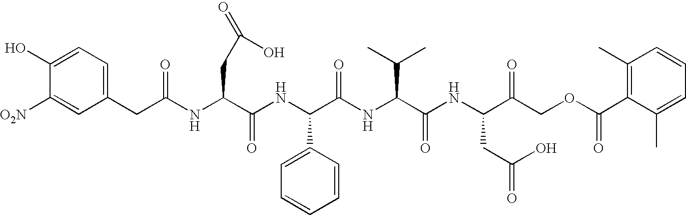 | 805.8 |
| AB14 | Np-D-E- 34-35-D- DMBA | 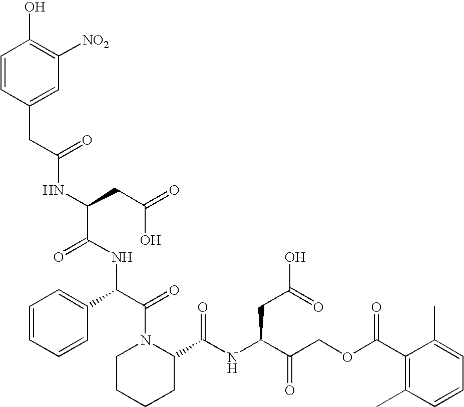 | 817.8 |
| AB15 | Np-26-34- V-D-DMBA | 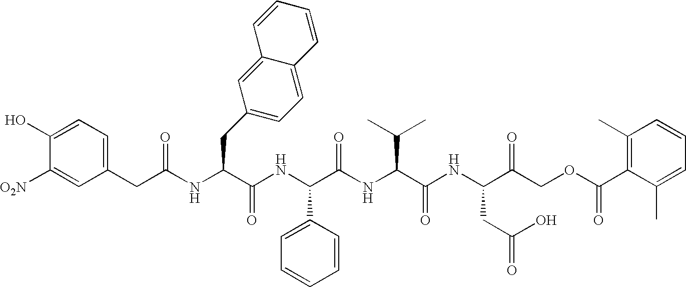 | 887.9 |
| AB16 | Np-26-3- V-D-DMBA | 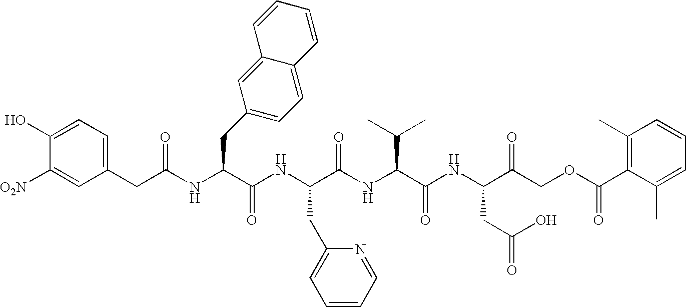 | 902.9 |
| AB17 | Np-26-E- V-D-DMBA | 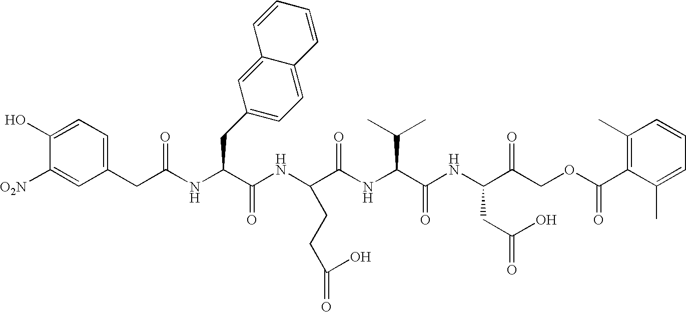 | 883.9 |
| AB18 | NP-31-E- T-D-DMBA | 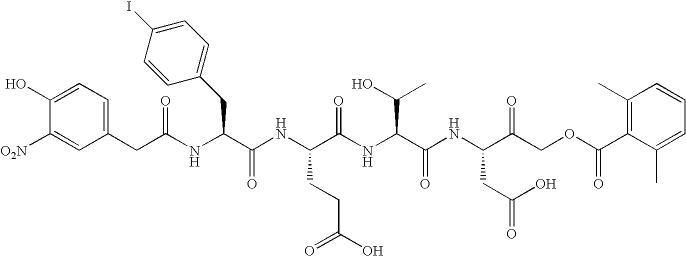 | 961.7 |
| AB19 | NP-31-E- 23-D-DMBA | 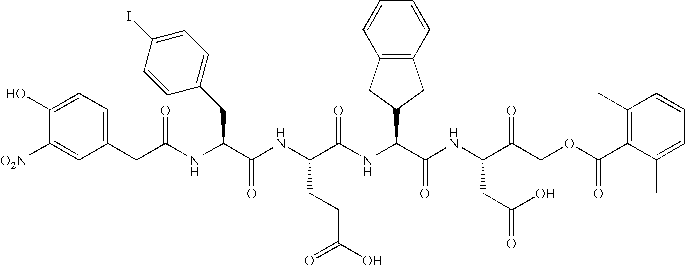 | 1034 |
| AB20 | Np-29-E- T-D-DMBA | 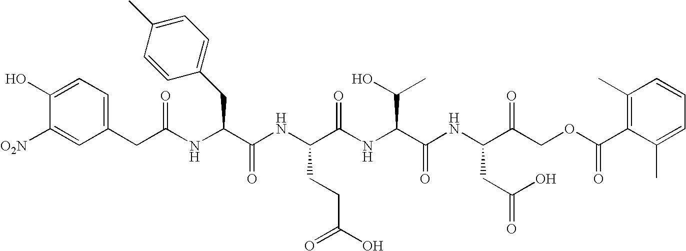 | 849.8 |
| AB21 | Np-32-E- T-D-DMBA | 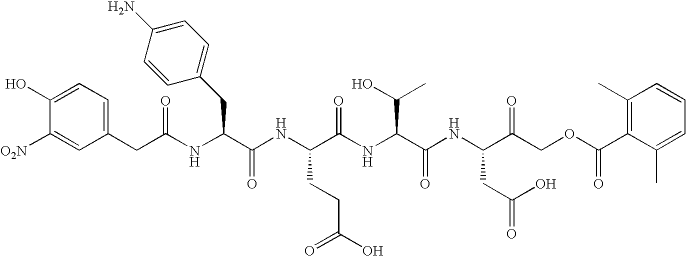 | 850.8 |
| AB22 | Np-21-41- 35-D-DMBA | 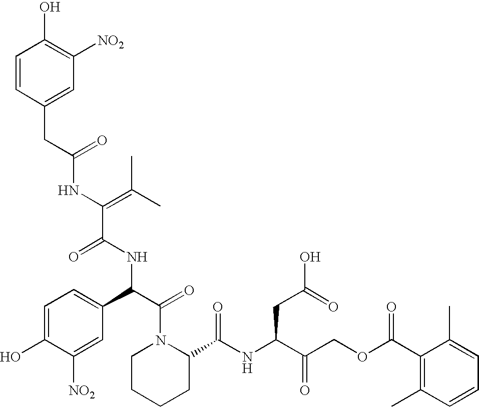 | 860.8 |
| AB23 | Np-21-40- 35-D-DMBA | 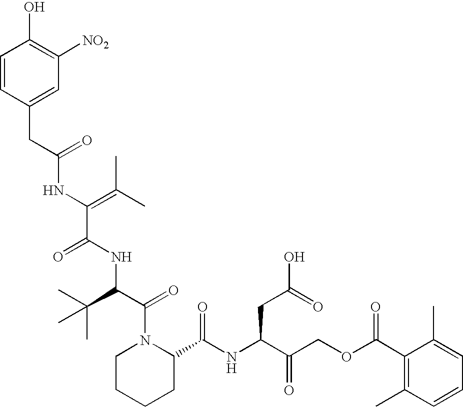 | 779.8 |
| AB24 | Np-21-41- 18-D-DMBA | 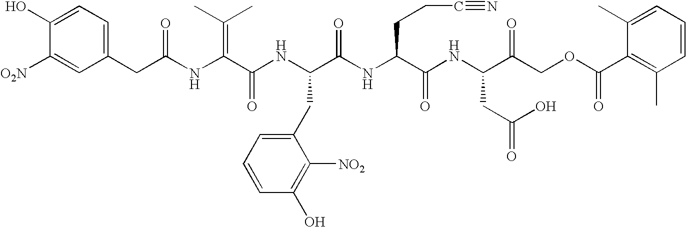 | 873.8 |
| AB25 | Np-31-41- 35-D-DMBA | 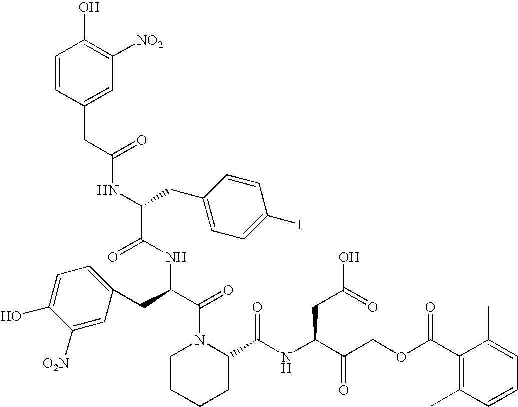 | 1051 |
| AB26 | Np-31-41- A-D-DMBA | 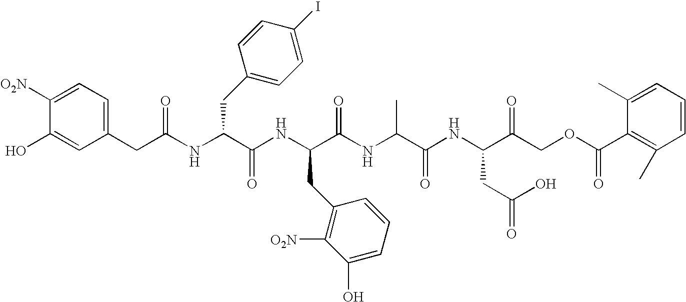 | 1011 |
| AB27 | Np-6-E- 35-D-DMBA | 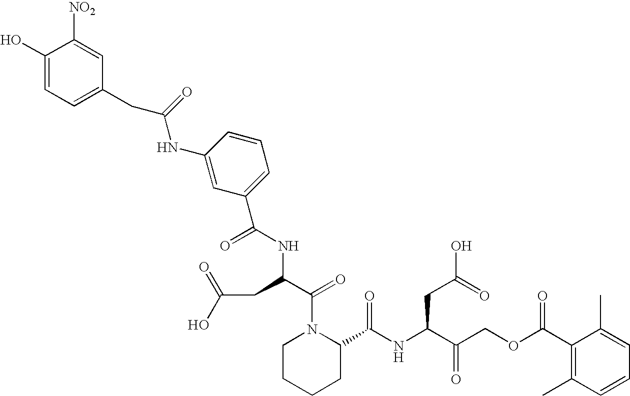 | 803.8 |
| AB28 | NH2-6-E- 8-D-DMBA | 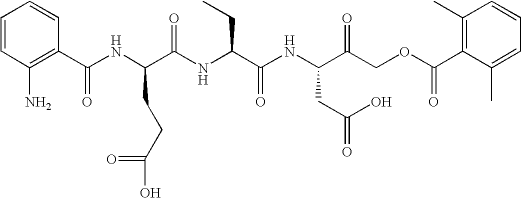 | 612.6 |
| AB29 | Np-D-E- 11-D-DMBA | 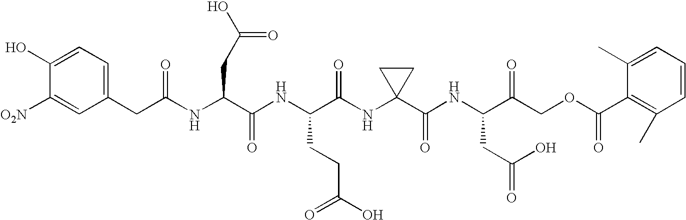 | 785.7 |
| AB30 | Np-D-30- 11-D-DMBA | 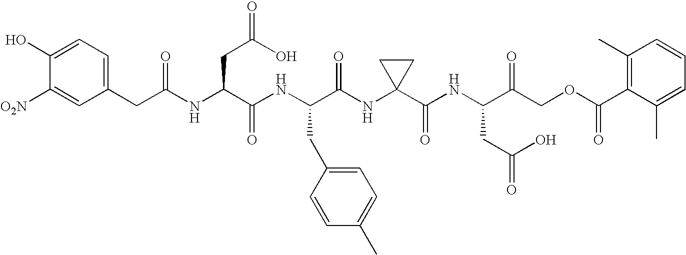 | 817.8 |
| AB31 | Np-D-30- V-D-DMBA | 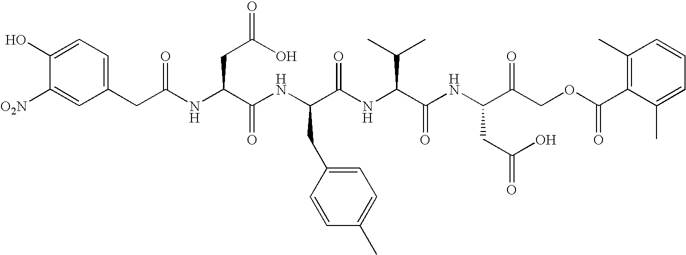 | 833.8 |
| bAB32 | bio-hex-27- 1-35-D- DMBA | 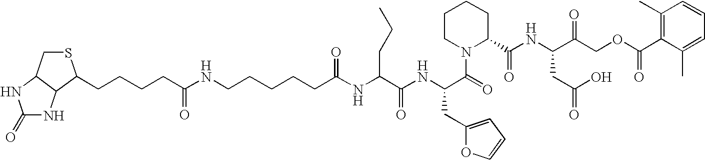 | 966.2 |
| bAB33 | bio-hex-27- | Not purified | |
| | 30-35-D- | | |
| | DMBA | | |
| bAB34 | bio-hex-27- 34-35-D- DMBA | 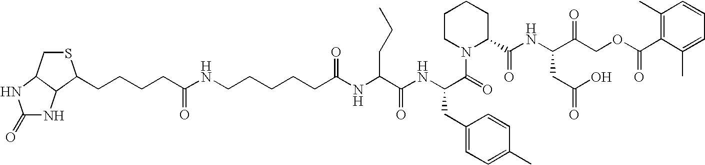 | 990.2 |
| bAB35 | bio-hex-27- 29-35-D- DMBA | 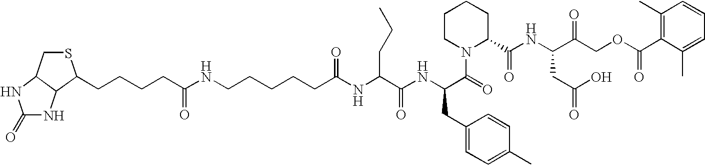 | 990.2 |
| AB37 | Cbz-3-V- D-AOMK | 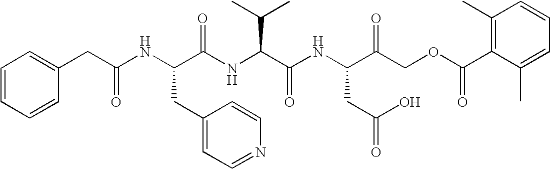 | 644.7 |
| AB38 | Np-P-L- A-D-AOMK | 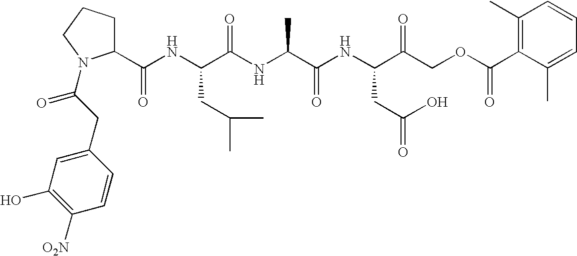 | 739.8 |
| AB39 | Np-P-I- E-D-AOMK | 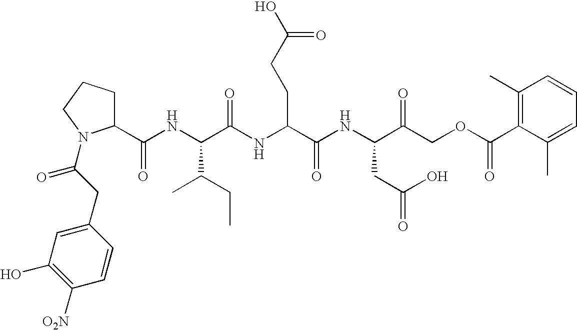 | 797.3 |
| AB40 | Np-I-L- A-D-AOMK | 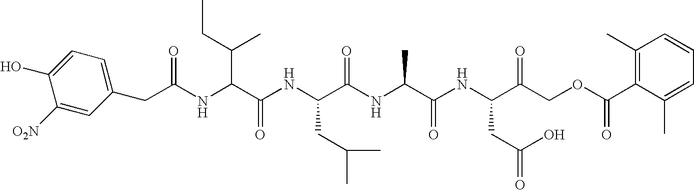 | 755.3 |
| AB41 | Np-I-L- 38-D-AOMK | 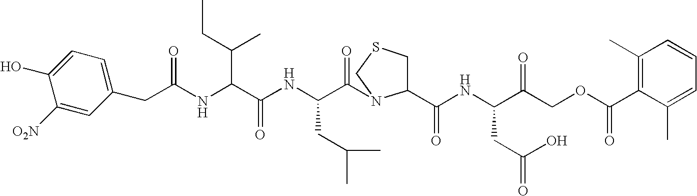 | 799.3 |
| AB42 | NP-I-F- P-D-AOMK | 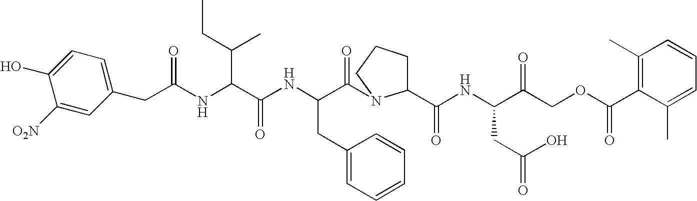 | 815.3 |
| AB43 | Np-39-L- A-D-AOMK | 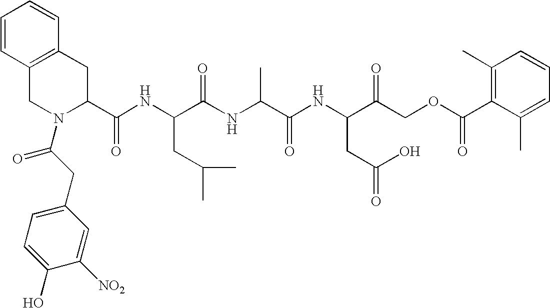 | 801.3 |
| AB44 | Np-36-L- A-D-AOMK | 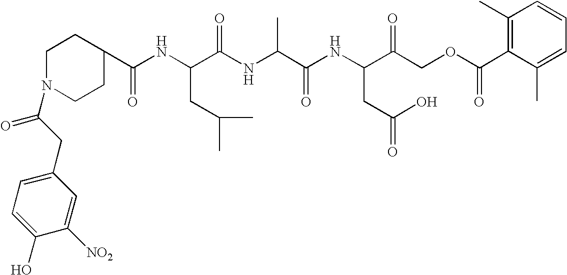 | 753.3 |
| AB45 | Np-E-V- D-AOMK | 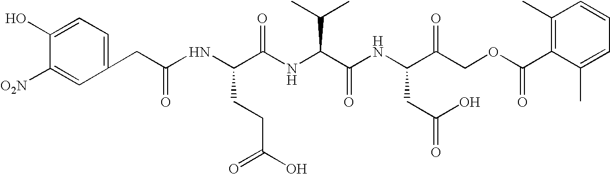 | 686.2 |
| AB46 | Np-E-8- D-AOMK | 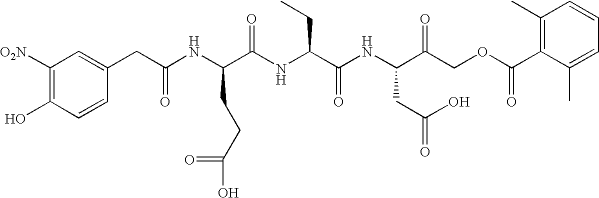 | 672.6 |
| AB47 | Np-W-E- H-D-AOMK | 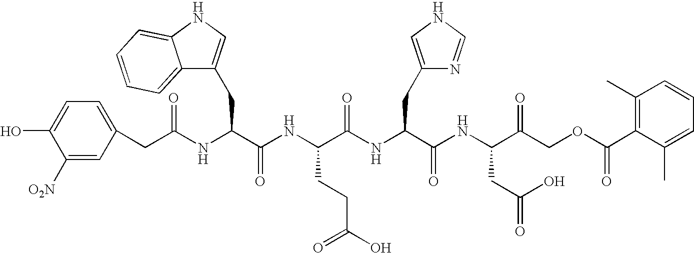 | 910.9 |
| AB48 | Np-W-E- 8-D-AOMK | 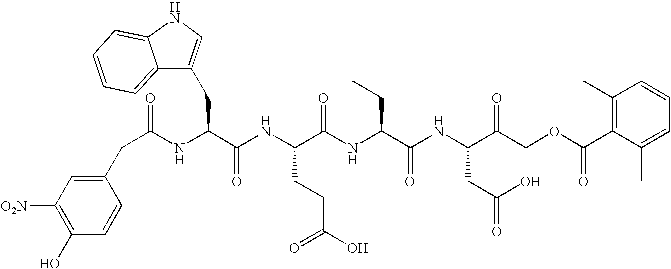 | 858.9 |
| AB49 | NP-P- D-AOMK | 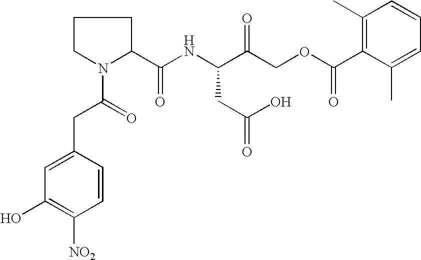 | 555.6 |
| AB50 | NP-E-P- D-AOMK | 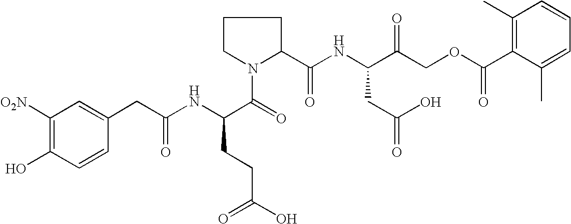 | 684.7 |
| AB51 | Np-3-P- D-AOMK | 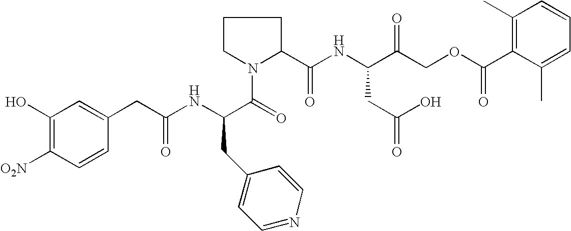 | 703.7 |
| AB52 | E-8-D | 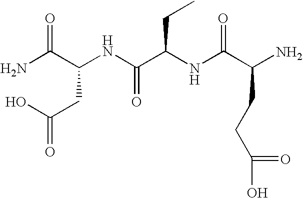 | |
| AB53 | NP-16-P- D-AOMK | 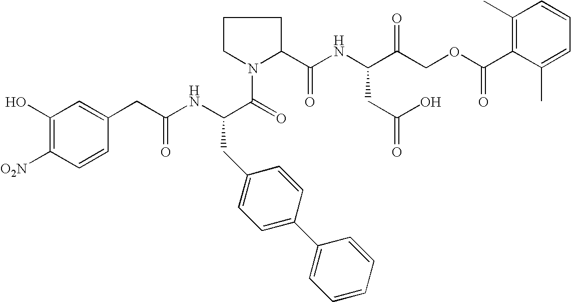 | 778.8 |
| AB54 | Np-25-P- D-AOMK | 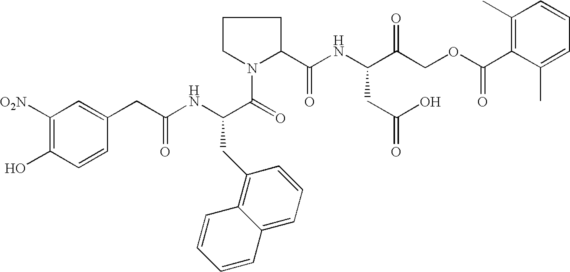 | 752.8 |
| AB55 | Np-26-P- D-AOMK | 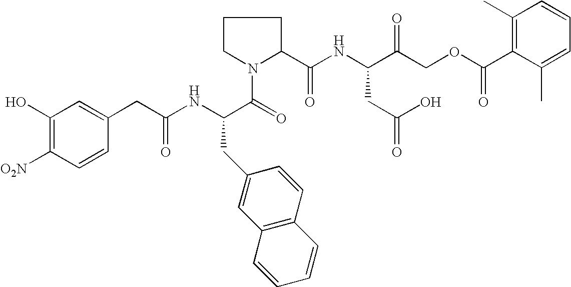 | 752.8 |
| AB56 | Np-F-P- D-AOMK | 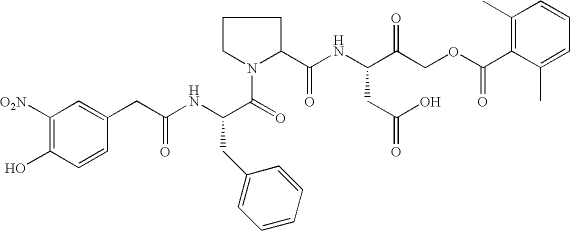 | 702.7 |
| AB57 | Np-19-P- D-AOMK | 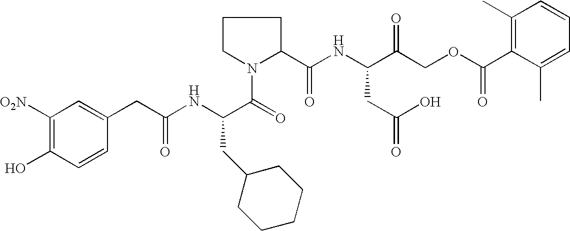 | 708.8 |
| AB58 | Np-17-P- D-AOMK | 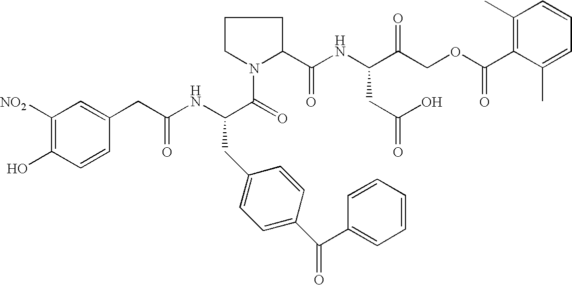 | 806.8 |
| AB59 | Np-31-P- D-AOMK | 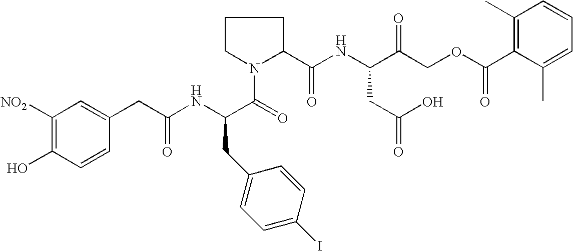 | 828.6 |
| AB60 | Np-para- Bromo-phe- P-D-AMOK | 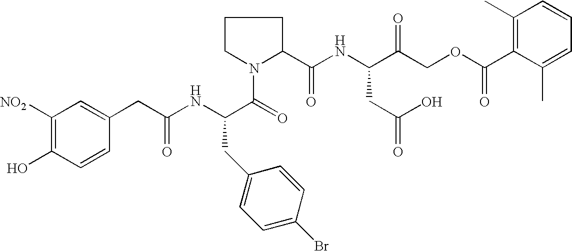 | 781.6 |
| AB61 | Np-para- nitro-phe- P-D-AMOK | 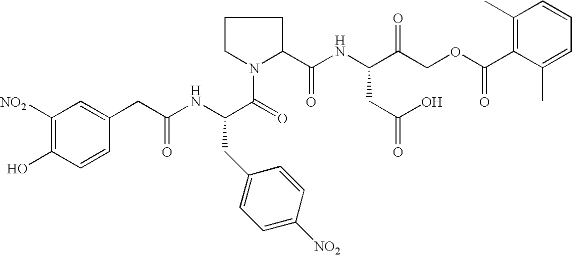 | 747.7 |
| AB62 | Np-para- chloro-phe- P-D-AMOK | 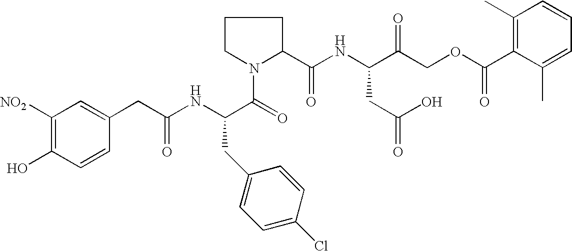 | 737.2 |
| AB63 | Np-4- methyl-Phe- P-D-AMOK | 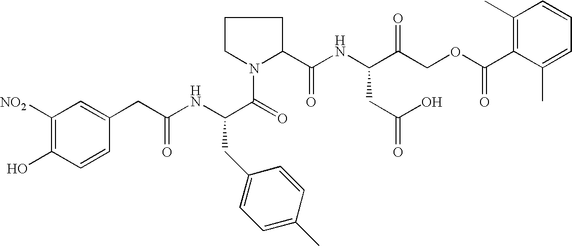 | 716.7 |
| AB64 | Np-4- styryl- alanine- P-D-AMOK | 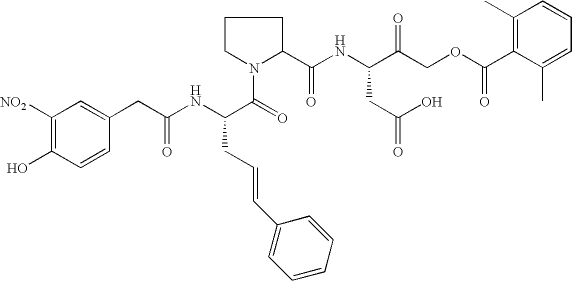 | 761.8 |
| AB65 | 4-[2-(Boc- amino) ethoxy]-L- phenyl- alanine- P-D-AOMK | 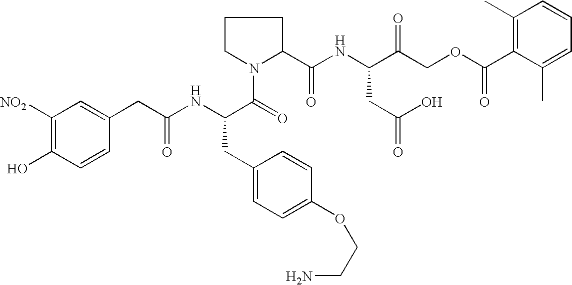 | |
| AB66 | NP-23-P- D-AOMK 23 = Igl | 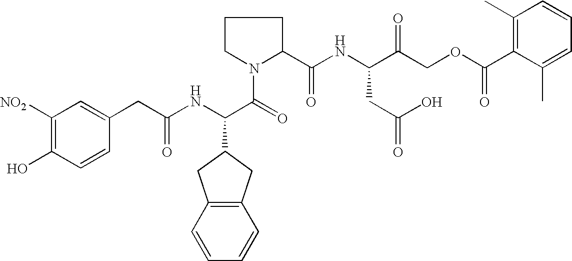 | 728.7 |
| AB67 | Np-L- homoCha- P-D-AMOK | 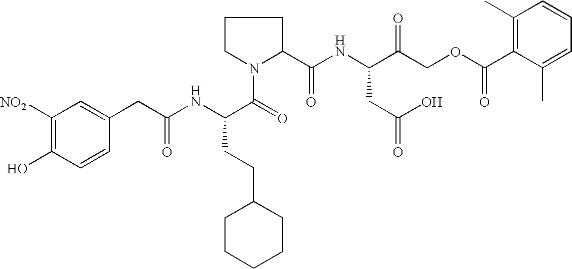 | 722.3 |
| AB68 | Np-4- (tert- butoxy- carbonyl- methoxy)- L- phenyl- alanine- P-D-AOMK | 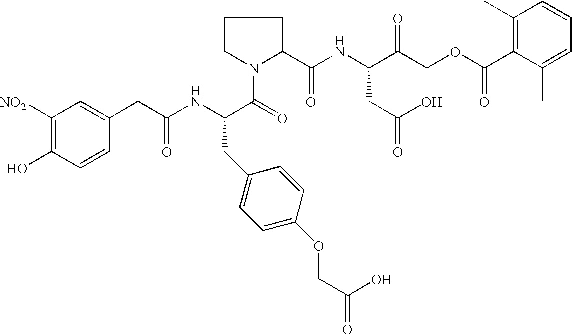 | 776.6 |

### Naming Conventions

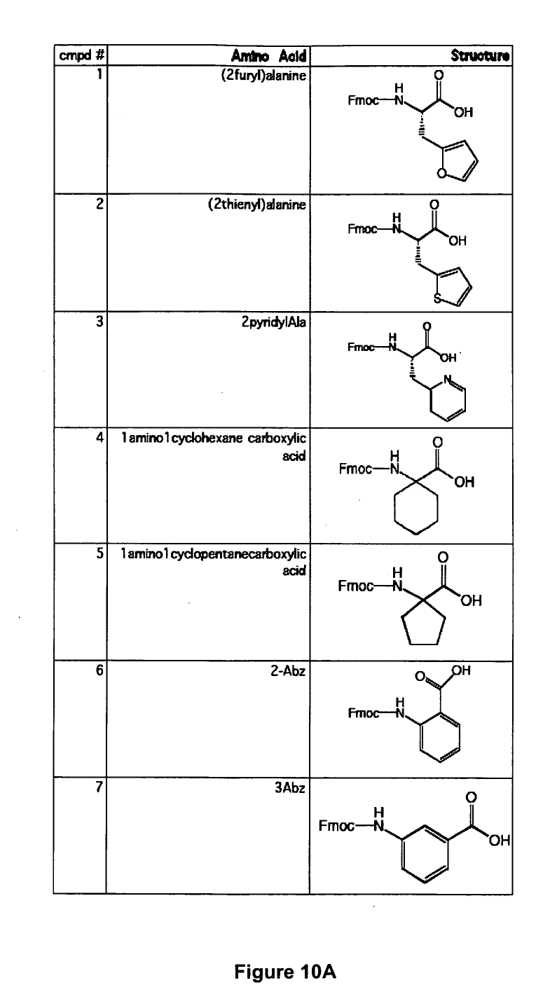

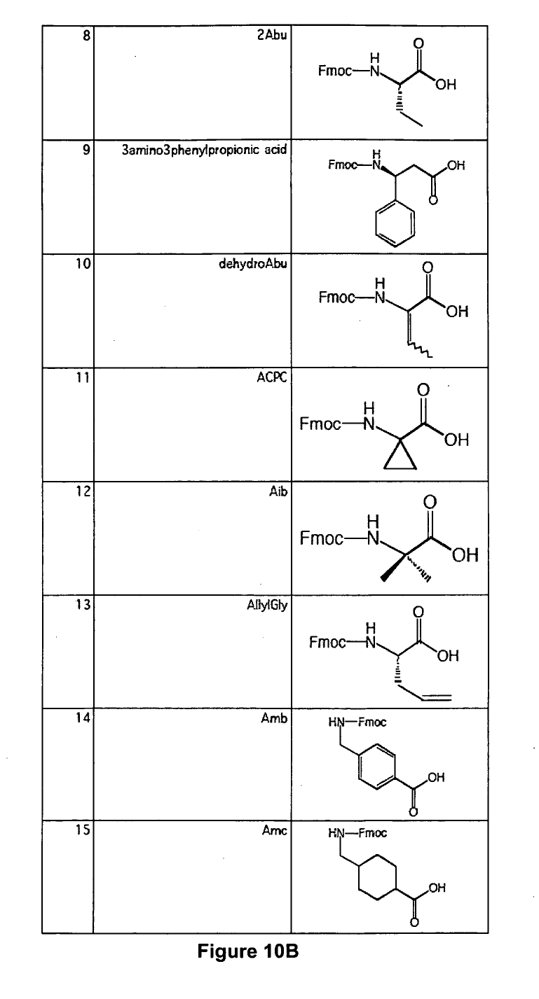

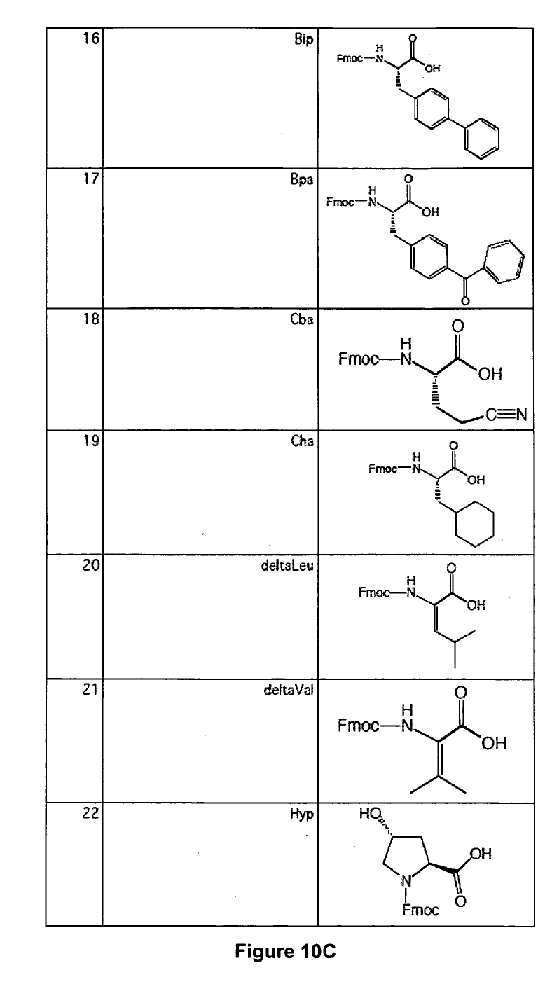

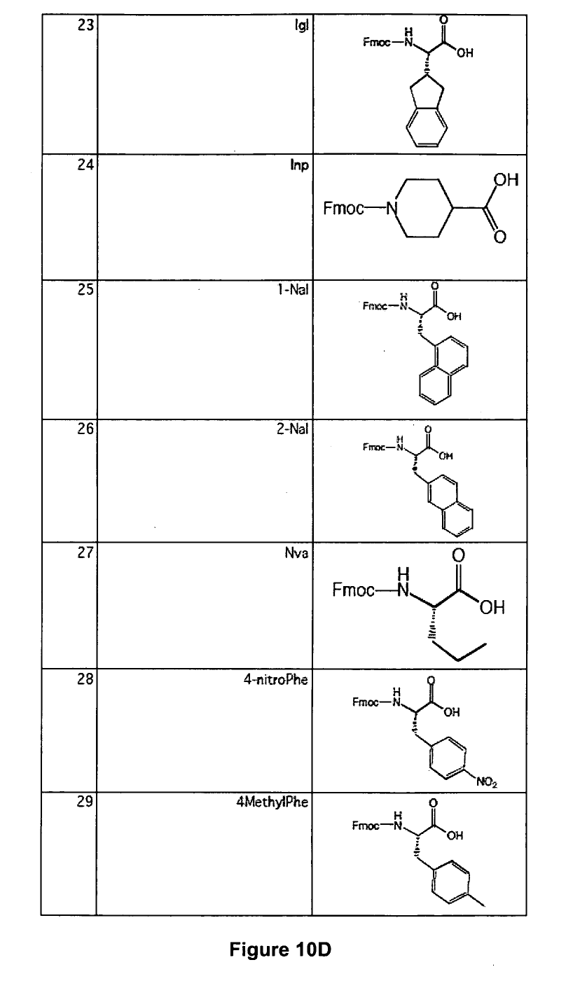

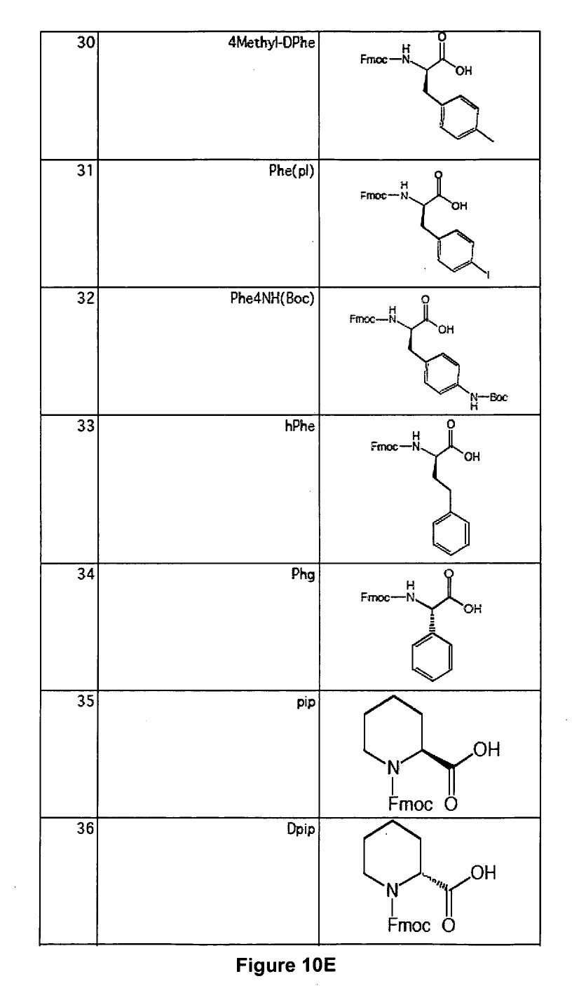

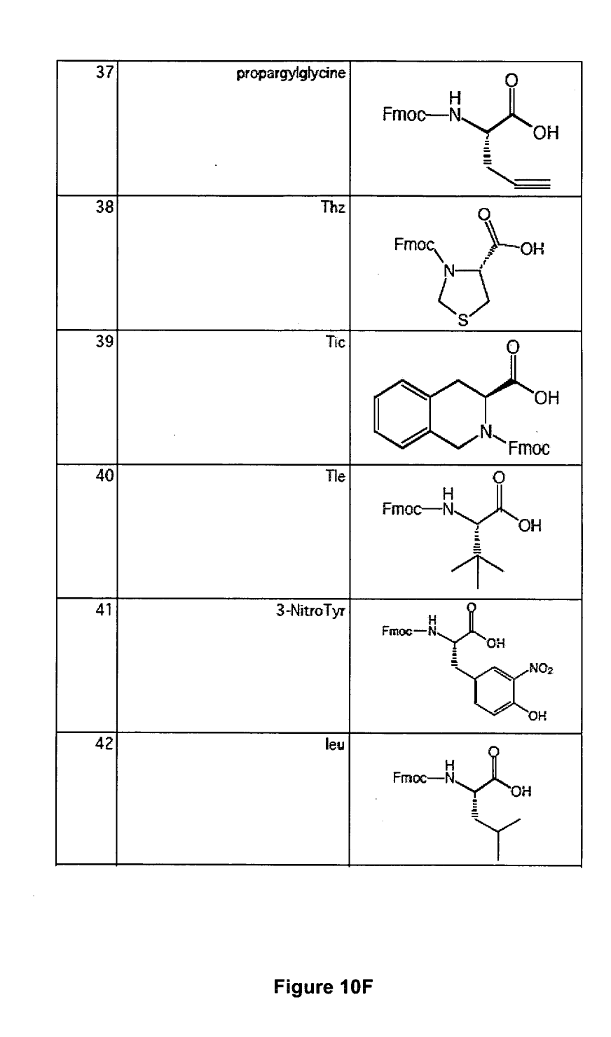

The exemplary compounds described above are identified in the first column, headed “Short Name,” by an arbitrary designation beginning with “AB.” In the second column of the above table, headed “Compound Name,” a group listing, such as “Cbz-E-8-D-DMBA,” seen for AB46 immediately above, is given. In this notation, Cbz refers, as is known, to benzyl carbamate (cf. “Np” or nitrophenol), E refers to the standard amino acid code for glutamate, 8 refers to non-natural amino acid #8 in FIG. 10, D refers to the standard single letter amino acid code for aspartate and DMBA refers to the dimethyl benzoic acid cap. The structures may be read from left to right, R1, P3, P2, (aspartate) and R2. In some instances, the structures contain only R1, P3, P2, aspartate and R2 (Formula II). Otherwise, the naming contains R1, P4, P3, P2, R2.

### DEFINITIONS

All terms used herein are used in their generally accepted scientific sense unless specifically defined other wise.

The term “caspase” is used in its generally accepted sense, i.e., the “c” refers to a cysteine protease mechanism, and “aspase” refers to the group's ability to cleave aspartic acid, the most distinctive catalytic feature of this protease family. Each of these enzymes is synthesized as a proenzyme, proteolytically activated to form a heterodimeric catalytic domain. Group I caspases are involved in the inflammatory response and similar pathways. Group II caspases are upstream/apical caspases and critical components in the apoptosis signaling pathway. Group III caspases are downstream/effector caspases. Caspases cleave C-terminal to an aspartic acid residue in a polypeptide and are involved in cell death pathways leading to apoptosis (see Martin and Green, Cell 82:349-352 (1995)). The caspases previously were referred to as the “Ice” proteases, based on their homology to the first identified member of the family, the interleukin-1β. (IL-1 beta) converting enzyme (Ice), which converts the inactive 33 kiloDalton (kDa) form of IL-1 beta to the active 17.5 kDa form. The Ice protease was found to be homologous to the Caenorhabditis elegans ced-3 gene, which is involved in apoptosis during C. elegans development, and transfection experiments showed that expression of Ice in fibroblasts induced apoptosis in the cells (see Martin and Green, supra, 1995). Specific protein sequences for members of the caspase gene family are given in Wang et al., “Functional Divergence in the Caspase Gene Family and Altered Functional Constraints: Statistical Analysis and Prediction,” Genetics, Vol. 158, 1311-1320, July 2001 as follows: 1) casp-3, U13737 (human 3-), U13738 (human 3-B), U49930 (rat 3-), U58656 (rat 3-B), Y13086 (mouse), U27463 (hamster), AF083029 (chicken), D89784 (frog); (2) casp-7, U37448 (human), Y13088 (mouse), AF072124 (rat), U47332 (hamster); (3) casp-6, U20536 (human), AF025670 (rat), Y13087 (mouse), AF082329 (chicken); (4) casp-8, AF102146 (human), AF067841 (mouse); (5) casp-10, U60519 (human 10a), U86214 (human 10/b), AF111345 (human 10/d); (6) casp-9, U60521 (human); (7) casp-2, U13021 (human), U77933 (rat), Y13085 (mouse), U64963 (chicken); (8) casp-14, AF097874 (human), AJ007750 (mouse); (9) casp-1, X65019 (human), AF090119 (horse), L28095 (mouse), U14647 (rat), D89783 (frog ICE-A), D89785 (frog ICE-B); (10) casp-4, Z48810 S78281 (human); (11) casp-5, X94993 (human); (12) casp-13, AF078533 (human); (13) casp-11, Y13089 (mouse); (14) casp-12, Y13090 (mouse); (15) invertebrate caspase, P42573 (C. elegans CED-3), Y12261 (Drosophila melanogaster), U81510 (armyworm, Spodoptera frugiperda).

Additional polypeptides sharing homology with Ice and ced-3 have been identified and are referred to as caspases, each caspase being distinguished by a number. For example, the originally identified Ice protease now is referred to as caspase-1, the protease referred to as caspase-3 previously was known variously as CPP32, YAMA and apopain, and the protease now designated caspase-9 previously was known as Mch6 or ICE-LAP6. The caspase family of proteases are characterized in that each is a cysteine protease that cleaves C-terminal to an aspartic acid residue and each has a conserved active site cysteine comprising generally the amino acid sequence QACXG, where X can be any amino acid and often is arginine. The caspases are further subcategorized into those that have DEVD cleaving activity, including caspase-3 and caspase-7, and those that have YVAD cleaving activity, including caspase-1.

The caspases are generally classified in family C14, but an inhibitor as described below may inhibit selectively a member of a similar family, such as C14, which is legumain. These families are part of a general class of Cysteine Peptidases (see http://www.expasy.org for classifications). For example, the selective inhibitor in certain cases is selective to one family member only, and, in certain embodiments, described below, may target legumain and a caspase, but only when a caspase is activated. When caspase is not activated, only legumain is targeted.

As can be determined by the description below, the term “selective cysteine protease inhibitor” refers to an inhibitor which binds to and inhibits an activated cysteine protease of a specific family member, e.g., caspase 3 (EC 3.4.22.56), caspase 7 (EC 3.4.22.60), caspase 8 EC 3.4.22.61), caspase 9 (EC 3.4.22.62), legumain (EC 3.4.22.34), etc. and not generically other cysteine proteases. Certain inhibitors may be specific for more than one family member. Certain inhibitors may have lesser activity for other proteases, but the primary target will be one or more of caspase 3, 7, 8 or 9. Off target inhibition will be generally no more than about ⅕ of the activity against the specific caspase.

As used herein, the term “amino” refers to a monovalent group of formula —NR³₂ where each R³ is independently a hydrogen, alkyl, or aryl group. In a primary amino group, each R³ group is hydrogen. In a secondary amino group, one of the R³ groups is hydrogen and the other R³ group is either an alkyl or aryl. In a tertiary amino group, both of the R³ groups are an alkyl or aryl.

As used herein, the term “aminocarbonyl” refers to a monovalent group of formula —(CO)NR⁴₂ where each R⁴ is independently a hydrogen, alkyl, or aryl.

As used herein, the term “aromatic” refers to both carbocyclic aromatic compounds or groups and heteroaromatic compounds or groups. A carbocyclic aromatic compound is a compound that contains only carbon atoms in an aromatic ring structure. A heteroaromatic compound is a compound that contains at least one heteroatom selected from S, O, N, or combinations thereof in an aromatic ring structure.

As used herein, the term “aryl” refers to a monovalent “aromatic” (including heteroaromatic) carbocyclic radical. The aryl can have one aromatic ring or can include up to 5 carbocyclic ring structures that are connected to or fused to the aromatic ring. The other ring structures can be aromatic, non-aromatic, or combinations thereof. Examples of aryl groups include, but are not limited to, phenyl, biphenyl, terphenyl, anthryl, naphthyl, acenaphthyl, anthraquinonyl, phenanthryl, anthracenyl, pyrenyl, perylenyl, and fluorenyl. The term aryl includes “substituted aryl” groups in which ring carbon atoms have additional substituents, such as methyl or other lower alkyl, amine, sulfur oxy, hydroxyl or nitrogen containing groups. Specifically included is dimethyl benzyl, as illustrated here below. Also included specifically is 2-nitro, 3-hydroxy benzyl.

As used herein, the term “lower alkyl: refers to straight or branched chain alky compounds of C1-C10, optionally substituted with a hydroxyl, nitrogen, nitroxy, sulfhydryl or sulfide group.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

### Inhibitor Library Design

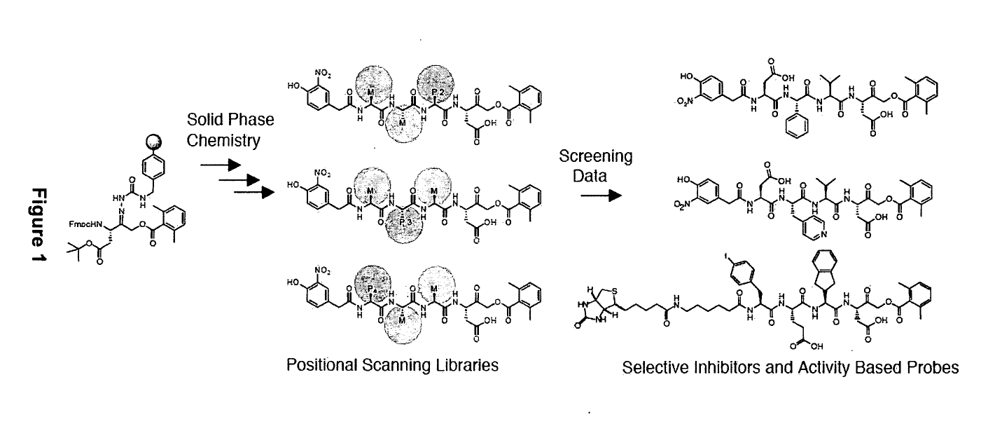

Initial libraries of 3-nitro-4-hydroxy phenyl acetyl (NP) capped tetra-peptide acyloxymethyl ketones were synthesized using solid phase methods originally developed by Ellman an co-workers (Amstad et al., 2001) and optimized for extended peptide AOMKs by our group (Kato et al., 2005) (FIG. 1). The robust nature of the solid phase synthesis method allowed the incorporation of a set of non-natural amino acids that contained a range of diverse hydrophobic, aromatic or bulky side chains. For all libraries, the P1 position directly adjacent to the reactive AOMK group was held constant as aspartic acid in order to satisfy the strict P1 specificity requirements of caspases (Stennicke et al., 2000). In all libraries one of the three remaining positions was held constant as a single natural (a total of 19 excluding cysteine and methionine plus norleucine) or non-natural amino acid (from a set of 41 non-naturals—see supplemental data) while the other positions contained mixtures of the natural amino acids. Thus, screening of all 60 amino acids was accomplished by the synthesis of three PSCLs composed of 180 sub-libraries that contained 361 compounds each. All inhibitors and probes contain the dimethylbenzoic acid acyloxymethylketone (AOMK) warhead that has been described as optimal for caspase-targeted ABPs (Kato et al., 2005; Thornberry et al., 1994).

As shown in FIG. 1, a representative compound solid phase chemistry was used to synthesize positional scanning combinatorial libraries (PSCLs) of nitrophenyl acetate (NP) capped peptide acyloxymethyl ketones (AOMKs). For all libraries, the P1 position directly adjacent to the reactive AOMK group was held constant as aspartic acid due to the strict cleavage requirements of caspases at this residue. One of the three remaining positions was also held constant (gray circles, M or P2, P3 or P4, top to bottom) as a single natural (a total of 19 excluding cysteine and methionine plus norleucine) or non-natural amino acid (from a set of 41 non-naturals—listed in FIG. 10) while the other positions contained isokinetic mixtures of the natural amino acids (gray circles around “M”). Single inhibitor compounds were selected after screening to determine the binding preference of individual caspases. Tags, such as biotin, were added in place of the nitrophenyl acetate cap of selective inhibitors to make activity based probes (ABPs).

Details of synthetic methods are given below in Methods and Materials.

### Inhibitor Library Screening Using Purified Recombinant Caspases

The complete set of PSCLs (positional scanning combinatorial libraries) of peptide AOMKs were screened in triplicate using a simple fluorogenic peptide substrate assay. Purified recombinant caspases-3, 8 or 9 were pre-incubated with inhibitor sub-libraries followed by addition of an optimal fluorescent substrate (DEVD-AFC, IETD-AFC, and LEHD-AFC for caspases 3, 8, and 9 respectively). Production of the fluorescent byproduct was measured at a set endpoint. Residual enzyme activity was calculated from the ratio of normalized fluorescence signal of inhibited and control non-inhibited samples. Caspase-7 was not used in the initial kinetic screen as it shares a common extended specificity with caspase-3 (Thornberry et al., 1997) thus making it likely that the overall specificity patterns would be similar to those observed for caspase-3.

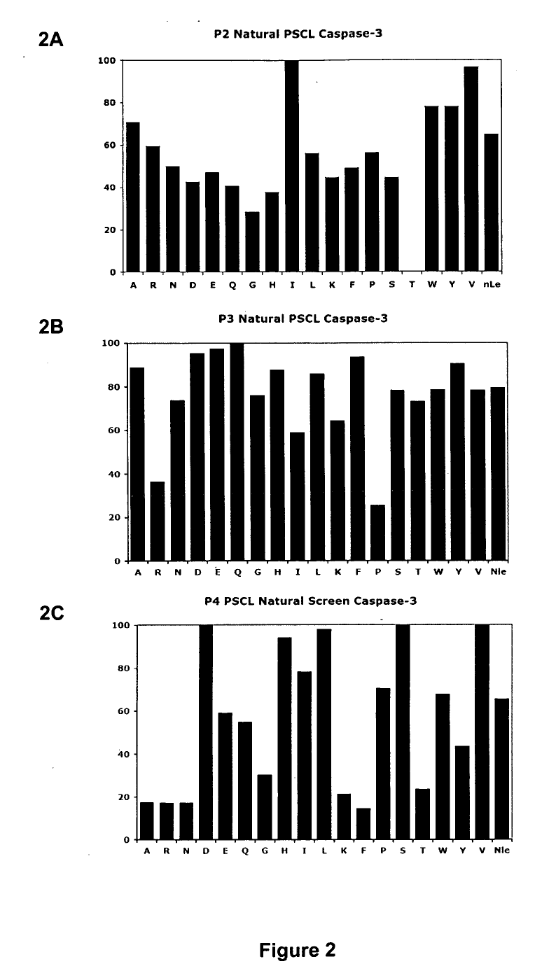

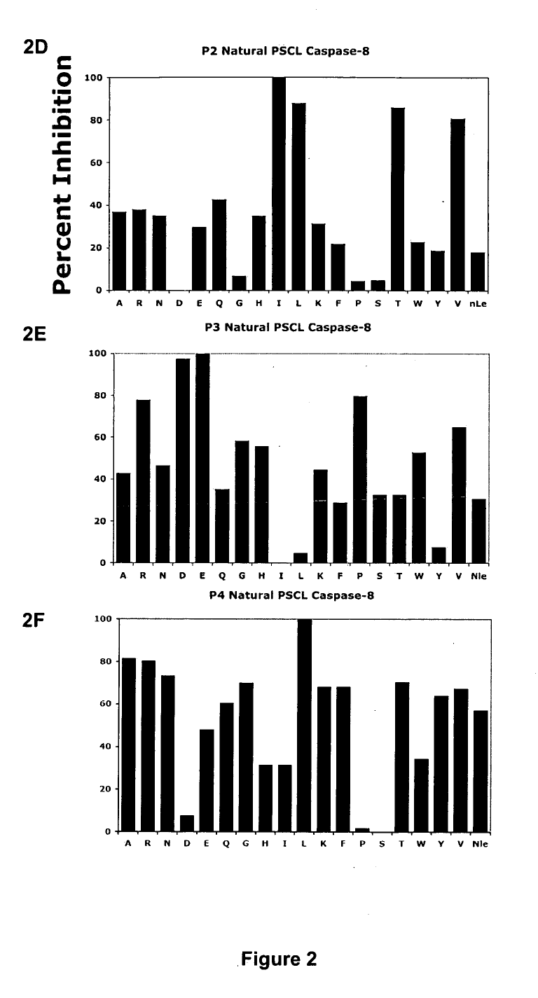

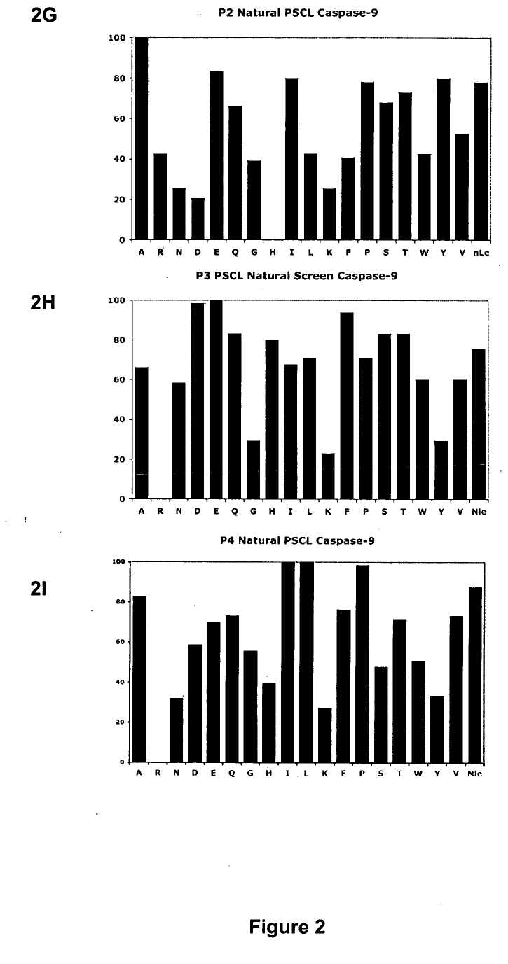

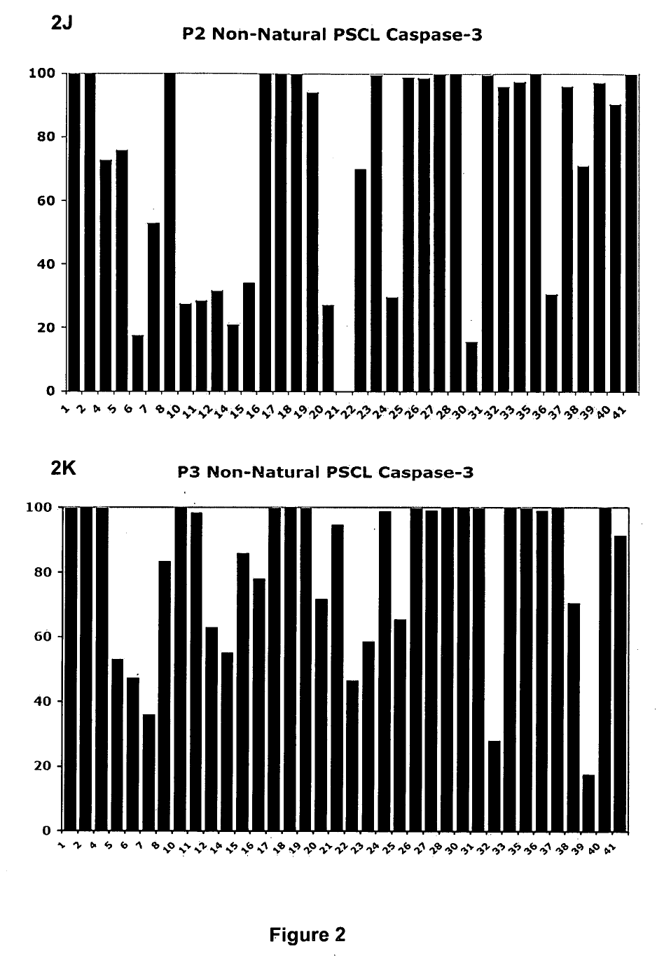

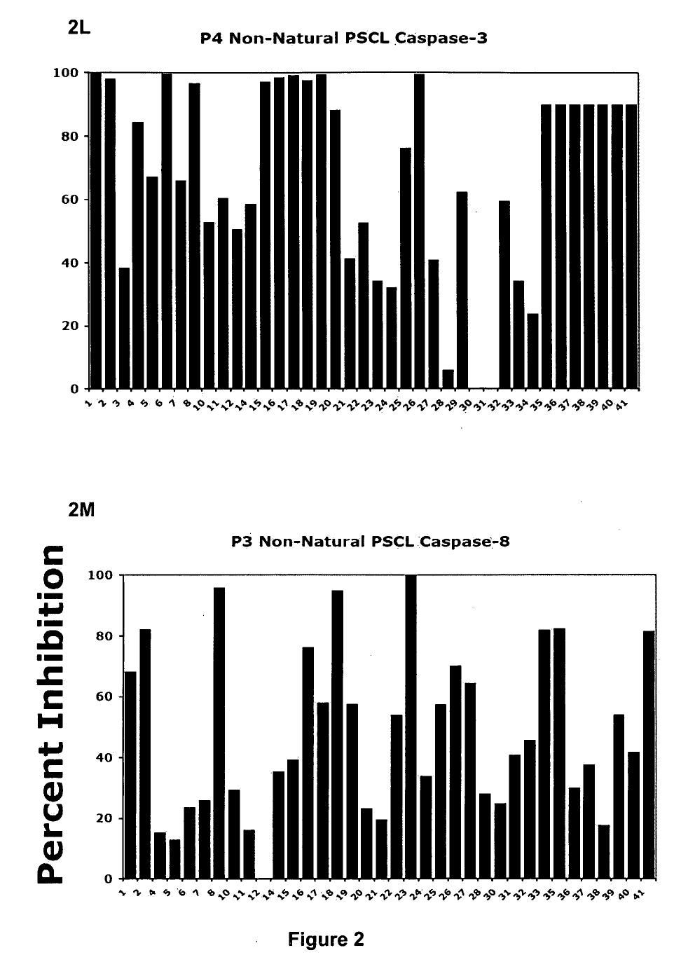

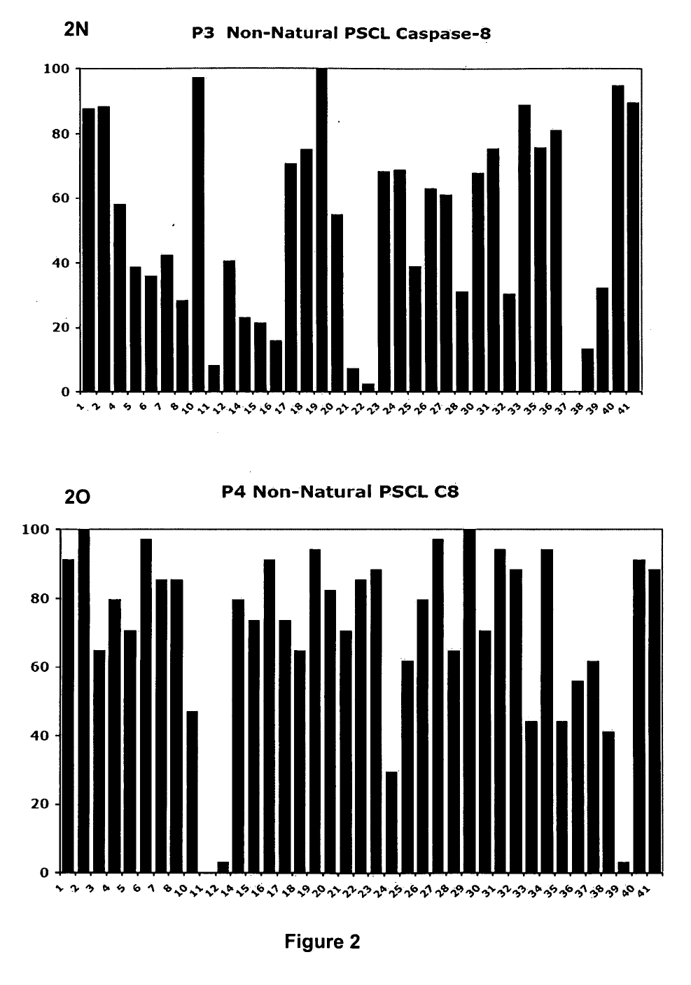

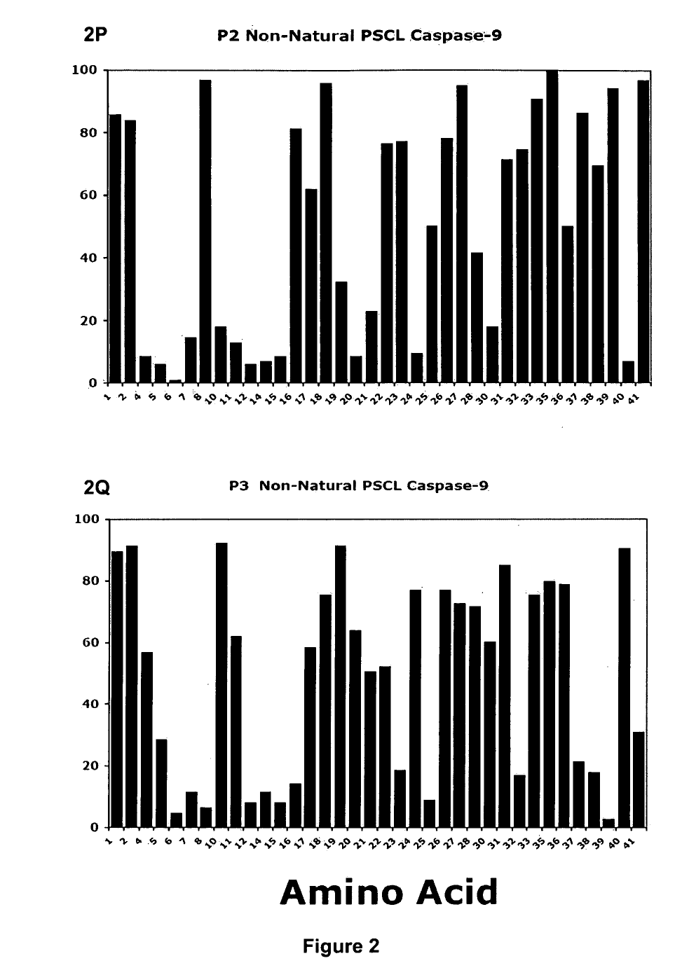

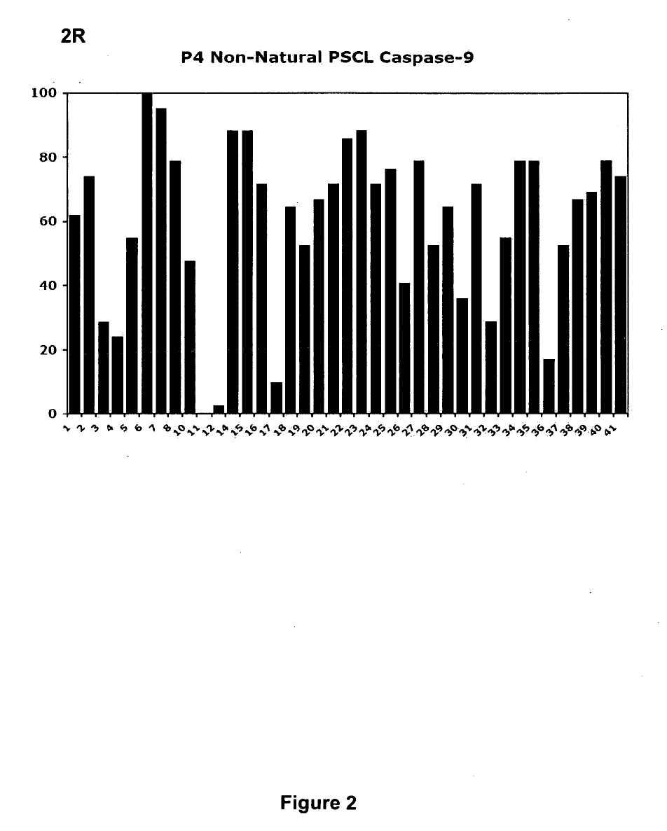

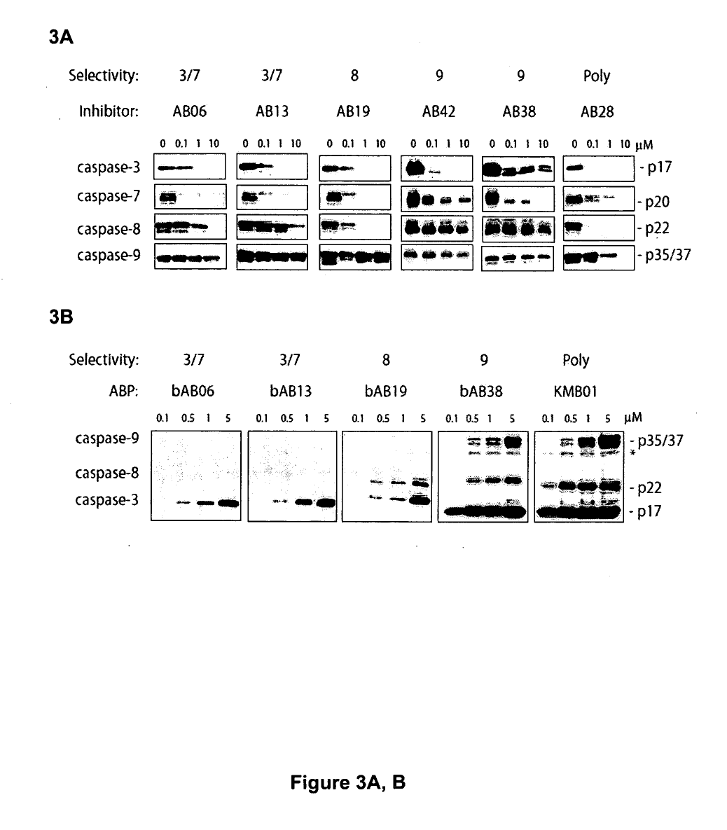

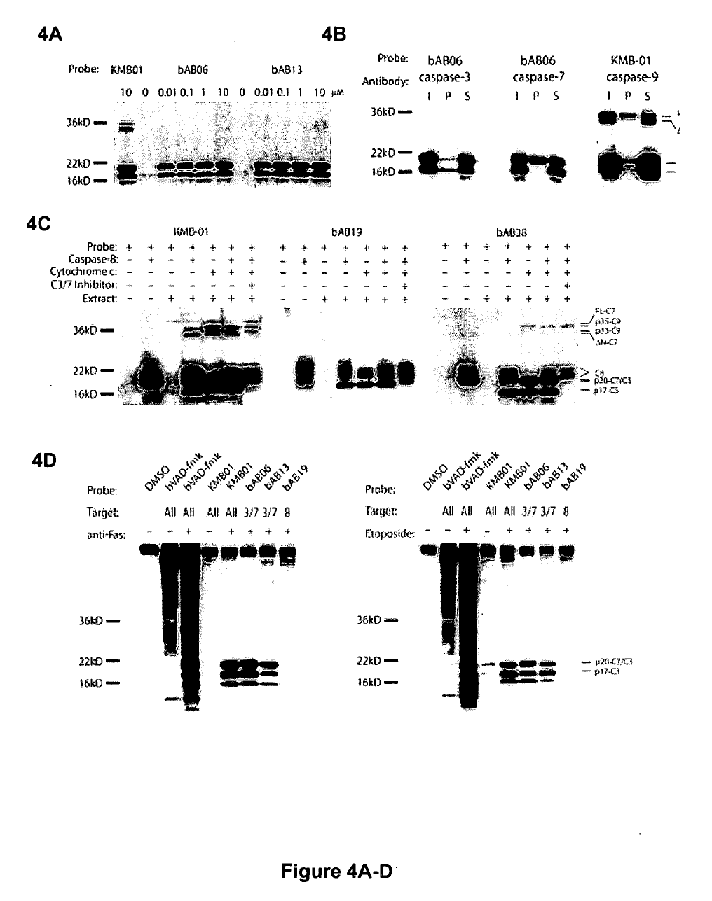

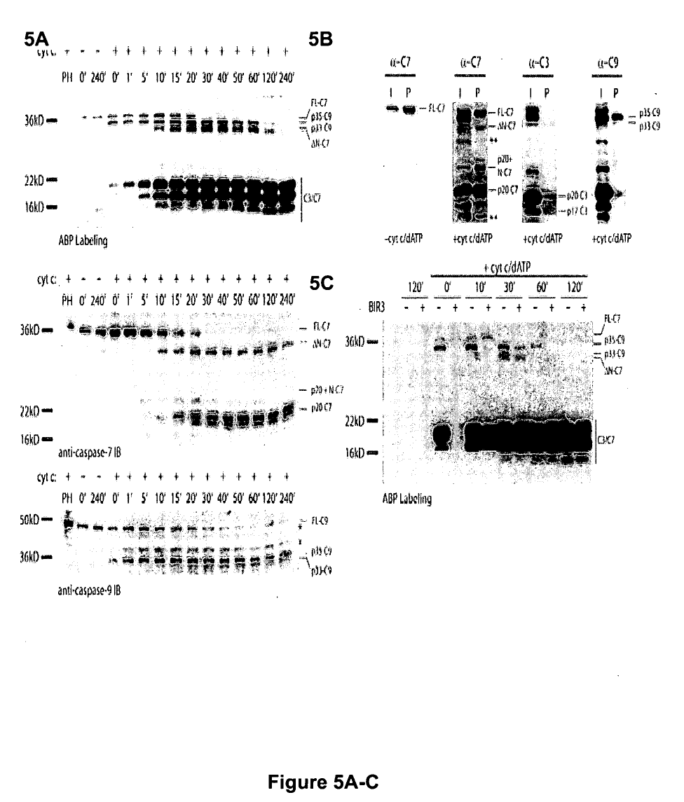

To aid in data analysis, residual enzyme activity values were organized using a hierarchical clustering algorithm (Eisen et al., 1998) that converts residual activity values into a color format, or heat map, where red and blue colors represent 0% and 100% residual activity respectively. Heat maps (represented as bar graphs in FIG. 2) were generated to provide “affinity fingerprints” (Greenbaum et al., 2002) of the preferred amino acids in the inhibitor specificity region for each of the caspases and were used to design selective inhibitors. Further details on this methodology may be found in Greenbaum et al., 2000; Greenbaum et al., 2002; Nazif and Bogyo, 2001.

Inhibitor specificity for the natural amino acid sub-libraries agreed very closely with previous reported substrate specificity data for the caspases (Thornberry et al., 1997). This suggests that the covalent AOMK-based inhibitors bind in manner similar to a substrate. However, the optimal histidine P2 identified by substrate library screening was not optimal in the context of the peptide AOMK. Further structural studies may help to explain the observed inhibitor specificity profiles.

Overall, several general specificity themes became apparent upon analysis of library screening data. Inhibitors containing alanine, proline and the proline-like non-natural amino acid 35 in the P2 position favored caspase-9 while amino acids containing aromatic rings such as non-naturals 3 and 34 in the P3 position directed specificity towards caspase-3 and away from caspases 8 and 9. Additionally, caspase-8 preferred phenylalanine analogs such as non-natural amino acids 29 and 31 in the P4 position and had a stricter requirement for glutamic acid in the P3 position relative to the other caspases. These general specificity themes were used to build optimal inhibitors and activity based probes.

### Design and Evaluation of Selective Inhibitors and Activity Based Probes

### Substrates

We initially set out to test the utility of reported optimal substrate sequences for the design of selective inhibitors. We therefore synthesized AOMK versions of the amino acid sequences reported for substrates that have also been used in a number of commercially available “selective” inhibitors.

### Inhibition Curves for AB07, AB08 and AB09

The inhibitors AB07 (NP-LEHD-AOMK, caspase-9), AB08 (NP-LETD-AOMK, caspase-8), and AB09 (NP-DEVD-AOMK, caspase-3) were synthesized and kinetic inhibition constants (Kiapp) were obtained for all compounds for caspases 3, 7, 8 and 9 using the progress curve method for pseudo-first order reactions kinetics (Salvesen, 1989) (structures shown in Table I). Further information is give in Table IV, which shows Ki(app) values for AB compounds. Ki(app) values (also called Kass or Kobs/I) represent the speed of inhibitor binding to a target enzyme. Units are [M-1s-1]. NI indicates no inhibition at concentrations tested, ND indicates data not determined, SD indicates standard deviation.

### Preparation of Selective Caspase Inhibitors

In general the compounds designed based on predicted optimal substrate sequences lacked selectivity. In particular NP-LEHD-AOMK, which was designed to target caspase-9 showed more rapid inhibition of caspase-8. Similarly, NP-DEVD-AOMK, which was designed to target caspase-3 showed strong activity towards caspase-8. These data highlight the lack of selectivity of the optimal natural peptide sequences and suggest that great care should be taken when using commercial “selective” inhibitors of the caspases.

One of the primary limitations of the PSCL approach is the inability of the libraries to predict the importance of collaborative binding interactions for multiple specificity sites on a given inhibitor. Thus, it is often difficult to combine specificity data from multiple sub-sites to create a single optimized compound with additive selectivity properties. This is particularly difficult when using non-natural amino acids in the context of peptides containing natural amino acids. Therefore, we chose to replace one, two, or all three of the P2-P4 positions in the parent substrate based sequences with optimal residues from our screening data. In addition we made use of residues that selected against binding to a subset of caspase targets thereby increasing selectivity.

Caspase 3 Inhibitors AB06, AB13, AB12

For caspase-3 we chose to focus on changes in the P3 position of the optimal DEVD sequence as there were a number of both natural and non-natural residues that were well tolerated by caspase-3 and 7 and not caspases 8 and 9. We selected non-natural amino acids NN29 (p-methyl phenylalanine), NN3 (2pyridylalanine) and NN34 (phenylglycine) to generate the sequences D3VD (AB06), D34VD (AB13) and D29VD (AB12). (See non natural (NN) amino acid structures in FIG. 10). We also attempted to replace the charged P4 aspartic acid with a bulky hydrophobic non-natural 26 (napthylalanine) in the hopes of improving cell permeability without loosing significant 3/7 selectivity. Interestingly, placement of NN3 and NN34 in the P3 position was sufficient to generate highly potent and selective caspase-3 and 7 inhibitors, while the use of NN29 in the P3 position and NN26 in the P4 position reduced selectivity by increasing reactivity with caspase-8. Thus, we selected AB06 and AB13 as our optimal caspase-3 inhibitors. Both of these compounds were converted to biotin-labeled probes (bAB06, bAB13) by replacement of the NP cap with a long-chain biotin moiety. Importantly, these probes retained their selectivity for caspase-3 and 7 (Table IV).

### Caspase 3 Inhibitors AB46, AB50 and AB53

A diverse positional scanning library with the core sequence NP-X-Mix-D-AOMK was screened against recombinant caspase-3 and RAW extracts to identify P3 residues that were optimal for caspase 3 but poor for legumain. The residue “X” represents a selected non-natural amino acid and “Mix” is a mixture of all natural amino acids minus cysteine and methionine and plus norleucine.

A heat map was created presenting the percent residual activity of legumain or caspase-3 after treatment with inhibitor sub-libraries. As is known in a heat map, red squares indicate 0% residual activity while blue squares represent 100% residual activity. Table VI represents activity found as a heat map, where R=red, B=Blue, and W=white, or approximately 50%.

TABLE VI

| NN | Legumain | Caspase-3 |
|---|---|---|
| 4 | B | B |
| 5 | B | B |
| 16 | R | B |
| 19 | R | B |
| 30 | B | B |
| 12 | W | R |
| 11 | R | R |
| 22 | R | R |
| 21 | B | R |
| 6 | B | R |
| 39 | B | R |
| 36 | B | R |
| 2 | R | R |
| 18 | R | R |
| 3 | R | R |
| 33 | R | R |
| 27 | R | R |
| 28 | R | R |
| 1 | R | R |
| 34 | R | R |
| 32 | R | R |
| 29 | R | R |
| 7 | R | R |
| 14 | R | R |
| 15 | R | R |
| 38 | R | R |
| 40 | R | R |
| 8 | R | R |
| 20 | R | R |
| 10 | R | R |
| 37 | R | R |
| 41 | R | R |
| 24 | R | R |
| 35 | R | R |
| 17 | R | W |
| 26 | R | W |
| 25 | R | B |
| 23 | R | W |
| 31 | R | W |

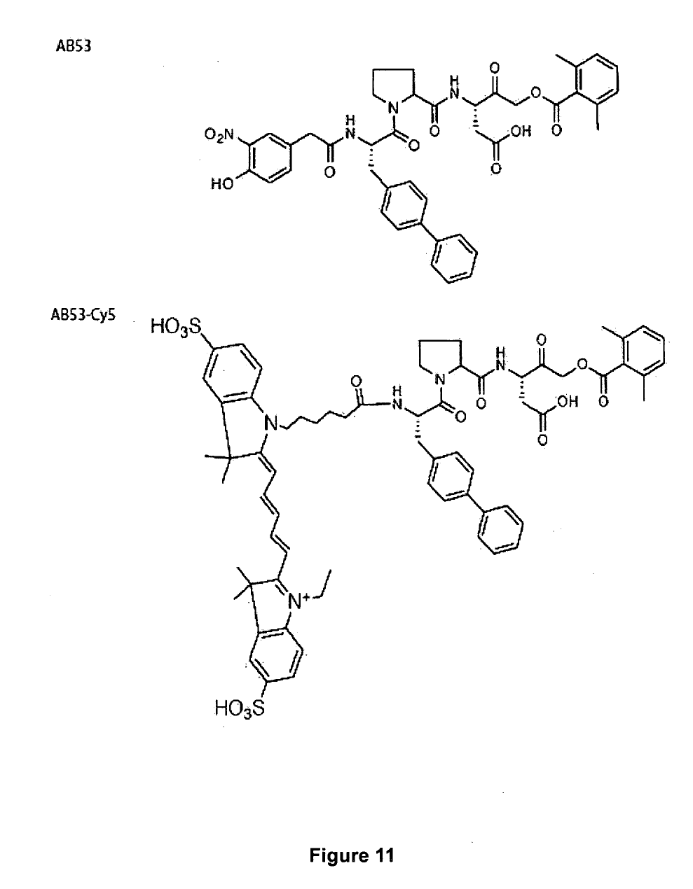

The most “blue” for legumain was clustered with the most “red” for caspase-3. Residue 16 (bolded) showed the lowest/highest activity against legumain and caspase-3, respectively, and is comprised in AB53 and AB53-Cy5. Also selectively active were 19 and 30. Inversely, bolded residues 6, 39 and 36 resulted in high inhibition of legumain and low inhibition of caspase-3. The percent residual activity of legumain was determined in RAW extract (RAW cells are macrophages with high cathepsins B and legumain activity) by pre-treating the extract with inhibitor sub-library and then adding the probe AB50-Cy5 to label residual legumain active sites. The extracts were then analyzed on a SDS-PAGE gel and scanned for fluorescence using a Typhoon scanner. The caspase-3 screen data was previously published and was used as a comparison to the legumain data (Berger et al, 2006). Non-natural amino acid 16 was found to give the most optimal specificity for caspase-3 (see above). AB53 incorporates the non-natural 16 into a scaffold containing a P2 proline previously found to direct selectivity away from Cathepsin B. AB 53 and AB53-cy (also highly active) are shown in FIG. 11.

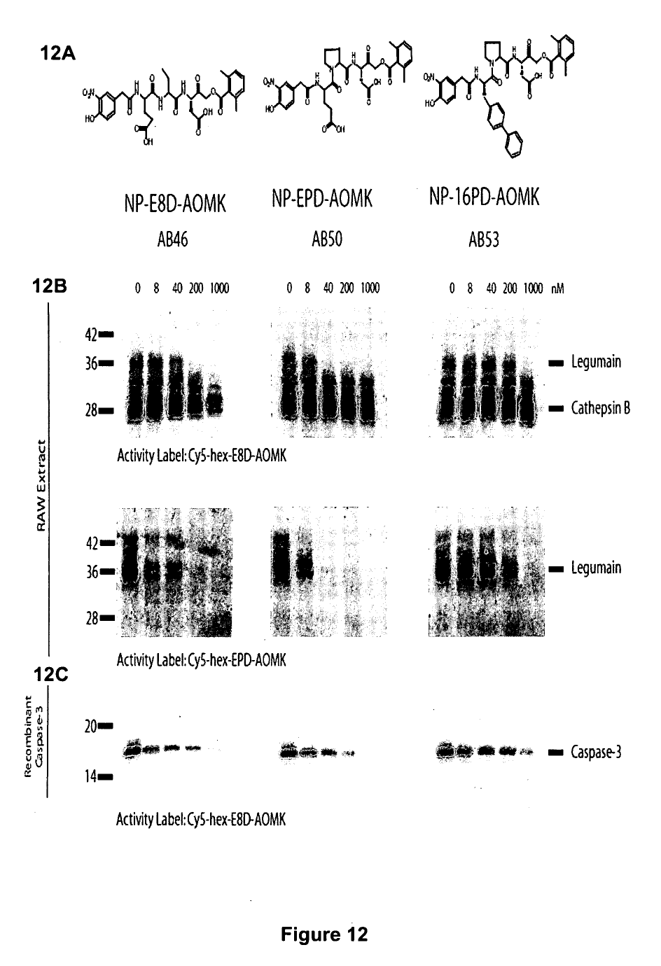

Referring now to FIG. 12, the specificity of AB46, AB50 and AB53 was measured by comparison of residual activity of cathepsin B, legumain and caspase-3 after treatment with varying concentrations of the compounds. Specificity for cathepsin B and legumain was evaluated in a macrophage (RAW) cell extract that contains high levels of both protease off targets (FIG. 12B). Activity against caspase-3 was tested using purified recombinant protein (FIG. 12C). In FIG. 12B, extracts are pre-treated with inhibitor and then labeled with AB46-Cy5 so cathepsin B labeling can be seen. The lower panel is the same experiment but with AB50-Cy5 (indicated as Cy5-hex-EPD-AOMK) labeling in order to emphasize the competition with legumain. These data show that inserting a proline into the P2 position of our inhibitors directs selectivity away from cathepsin B. Further addition of the non-natural non-natural 16 (biphenylalanine) into the P3 position we are able to direct selectivity away from legumain. All inhibitors have similar reactivity towards recombinant caspase-3 (FIG. 12C).

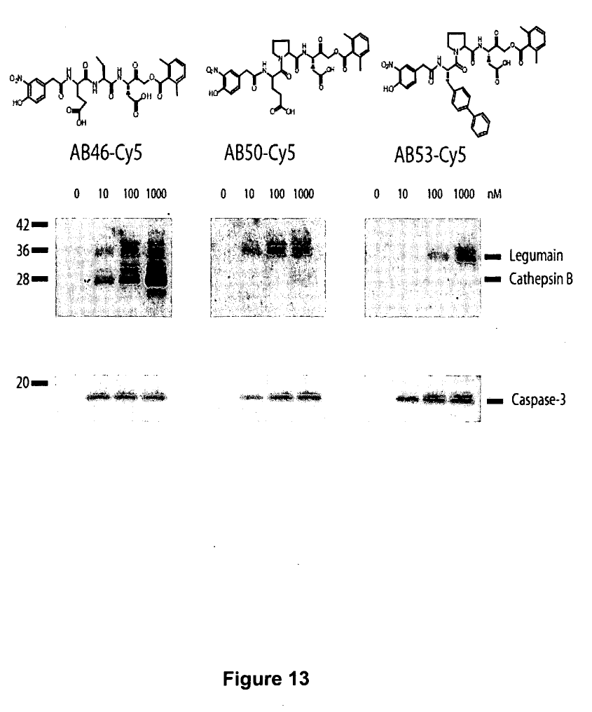

Cy5 fluorescently labeled versions of the three best caspase probes were compared in direct labeling experiments using recombinant caspase-3 (FIG. 13, bottom panels) and RAW cell extracts (FIG. 13, top panels). AB46-Cy5 labels legumain and cathepsin B, AB50-Cy5 only labels legumain, and AB53-Cy5 labels legumain only at the highest concentrations tested. All probes have similar reactivity towards recombinant caspase-3.

Caspase 8 Inhibitor AB20, AB19, bAB19

To design compounds that could discriminate between the intrinsic initiator caspase-9 and extrinsic initiator caspase-8 we initially chose to focus on the P2 and P4 positions because caspase-8 had a narrow selectivity preference in the P3 position. Substitution of the P4 Leu for the caspase-8 optimal NN29 resulted in a compound (AB20) with the highest kinetic inhibition constants for caspase-8 that we have measured so far. However this increase in potency came at the price of reduced selectivity relative to both caspase-9 and 3. Further substitution of the P2 position of AB20 with the non-natural 23 resulted in compound (AB19) that retained relatively good potency for caspase-8 and showed no measurable activity for caspase-9. While this compound still retained a reasonable level of activity against caspase-3, this was not a major concern as this cross-reactivity could be blocked by pre-treatment of a sample with a more selective caspase-3 inhibitor allowing specific labeling of caspase-8. AB19 therefore served as our optimal lead and was converted to the biotin labeled probe (bAB19) that retained its caspase8 selectivity (Table IV).

### Caspase 9 Inhibitors AB38, AB42

Development of caspase-9 selective inhibitors was much more challenging. The initial substrate-optimized sequence LEHD showed greater potency for caspase-8 than 9. We thus decided to begin by completely redesigning the peptide sequence using optimal natural amino acid residues in the P2-P4 positions. In addition NN38 was used in the P2 position as a result of its potentially high degree of selectivity for caspase-9 over caspase-8. In particular we focused on the P3 position since caspase-8 highly favors the acidic Glu residue in this position while caspase-9 tolerates a range of residues including leucine and phenylalanine. Of the 7 compounds selected as optimal caspase-9 inhibitors only a few showed activity against caspase-9 and many actually showed preference for caspase-8. This is most likely the result of vast differences in the overall catalytic efficiencies of the two enzymes. Recombinant caspase-9 is a generally weak enzyme leading to weak inhibition by small molecules. Thus, the most effective inhibitors still require high concentrations for complete inhibition resulting in cross reactivity with caspase-8. We therefore believe that screening of recombinant enzymes may not be optimal and a more thorough analysis using endogenous caspase-9 that has been activated in a cytosolic extract may be required to develop fully optimized caspase-9 selective inhibitors and probes. Nonetheless we were able to improve upon the current LEHD sequence and generate compounds with some degree of selectivity for caspase-9 (AB38 and AB42).

### General Caspase Inhibitors AB28, AB11

Finally, we selected several general caspase compounds based on optimal sequences for all caspases tested. Synthesis of the predicted optimal 6E8D sequence yielded only the free amino product (NH2-6E8D; AB28) due to difficulty in coupling of the NP cap to the hindered NN6 amino acid. However this free amine inhibitor (AB28) showed broad inhibition of all caspases. Similarly, the DEPD sequence (AB11) showed comparable inhibition kinetics to the previously reported general probe KMB01.

### Selectivity of Caspase Inhibitors and Activity Based Probes (ABPs) for Recombinant and Endogenous Caspases

Having selected a series of optimal caspase-3, 8 and 9 selective inhibitors we set out to further demonstrate overall selectivity and potency by indirect competition for labeling using a broad spectrum ABP to measure residual activity. We initially focused on recombinant caspase3, 7, 8, and 9. Each caspase was individually pre-incubated with our optimal series of selective inhibitors at a range of concentrations and residual activity was measured by addition of the general caspase probe KMB01. Overall, the competition data mirrored the kinetic data with caspase-3 and 8-specific compounds showing specific competition of the desired target.

This was shown by analysis of inhibitor and probe selectivity by indirect competition and direct labeling of recombinant caspases. Indirect competition of a panel of inhibitors with the general caspase probe KMB01 was carried out with individual caspases-3, 7, 8 and 9 (100 nM each), which were incubated with the specific inhibitors for 30 minutes followed by a 30-minute incubation with KMB01. Samples were analyzed by SDS-PAGE and residual active site labeling was visualized by biotin blotting using streptavidin-HRP. Also, the direct labeling of caspase active sites was carried out using specific ABPs. Equal amounts of active caspaseses-3, 8, and 9 (100 mM) were incubated together with increasing concentrations of each of the indicated biotinylated active site probes for 30 minutes. Active site labeling was visualized by SDS-PAGE analysis followed by biotin blotting using streptavidin HRP.

### AB06, AB13, AB19 and AB38 in Mixture of Recombinant Caspases

The caspase-9 specific compounds AB38 and AB42 showed a minimal degree of selectivity for caspase-9 over caspase-8 while the other caspase-9 specific compounds AB40 and AB41 showed no specific inhibition (Data not shown). The general inhibitor AB28 blocked labeling of all four caspases targets with caspase-9 requiring the highest concentration to obtain complete inhibition.

Based on competition and kinetic data, we synthesized biotinylated versions of the most promising selective inhibitors that included AB06, AB13, AB19 and AB38. To test the overall selectivity of our sequences and to determine their utility as selective activity based probes we monitored direct labeling of a mixture of recombinant caspases. Biotinylated probes were added at a range of concentrations to mixtures of recombinant caspase-3, 8, and 9 whose activities were normalized based on active site titration. Both bAB06 and bAB13 selectively labeled caspase-3 while bAB19 labeled caspase-8 with no labeling of caspase-9 even at concentrations as high as 10 μM, in the experiments described in the paragraph above. Not surprisingly, bAB38 labels both caspase-8 and caspase-9.

### Testing in Proteome

These encouraging results prompted us to assess the selectivity of our probes against endogenous caspase targets in a complex proteome. The intrinsic apoptosis pathway can be activated in cytosolic extract by addition of cytochrome c and dATP. This system allows temporal control of the apoptotic pathway and leads to activation of caspase-9 as well as caspase-3 and 7 (Liu et al., 1996). Upon activation of cell-free apoptosis for 10 min. the general probe KMB01 labeled a 35 kDa caspase-9 species and the two primary mature forms of caspase3 at 17 and 20 kDa as well the 33 kDa full length N-peptide processed and 20 kDa mature forms of caspase-7. The identities of these labeled species were confirmed by immunoprecipitation experiments using specific antisera as described in the paragraph below. The two caspase-3 selective probes bAB06 and bAB13 efficiently and selectively labeled the caspase-3 and 7 species at probe concentrations ranging from 10 nM to 10 μM.

Selective labeling of endogenous caspases in cell extracts and live cells with active site probes was carried out as follows: Hypotonic 293 cytosolic extracts were induced to undergo intrinsic apoptosis by addition of cytochrome c/dATP. KMB01, bAB06 and bAB13 were added 10 minutes after activation and labeling of caspase active sites was carried out for three minutes. Samples were analyzed by SDS-PAGE followed by biotin blotting using streptavidin-HRP. The identity of individual caspases was confirmed via immunoprecipitation using specific anti-sera for caspases 3, 7 and 9. Extracts (293) were activated with cytochrome c/dATP for 10 minutes, labeled by addition of indicated probes (100 nM final concentration for bAB06 and bAB13 and 10 μM final concentration for KMB01) and labeled caspases precipitated using specific anti-sera as described in Methods and Materials. Recombinant caspase-8 (100 nM) was either directly labeled or added to cell extracts (293) with or without cytochrome c/dATP activation and then labeled with the indicated probes (10 μM final concentration). The caspase-3 selective inhibitor AB06 (10 μM final concentration) was also added 10 minutes prior to probe addition to indicated samples. Labeling of caspases was monitored by SDS-PAGE followed by biotin blotting with streptavidin-HRP. Labeling of endogenous caspase-3 and 7 in intact Jurkat cells induced to undergo apoptosis through etoposide or anti-Fas treatment was carried out as follows: Cells (3×106) were incubated with apoptosis inducers for 15 hours and then labeled by incubation for an additional two hours with the panel of probes indicated. b-VAD-fmk (fluoromethyl ketone), KMB01 and bAB19 were used at 10 μM concentration final. bAB06 and bAB13 were used at 1 μM final concentration.

Thus, the caspase-3 selective probes proved to be valuable for use against endogenous caspase targets in complex proteomes.

Since cytosolic extracts induced to undergo intrinsic apoptosis through addition of cytochrome c/dATP do not contain detectable amounts of active endogenous caspase-8 we evaluated selectivity of the caspase-8 and 9 selective probes by adding exogenous active recombinant caspase-8 to the extracts in conjunction with cytochrome c/dATP activation. We used a concentration of active site titrated caspase-8 (100 nM) that was in the range of the reported endogenous level of active caspase-8 in Fas-receptor activated Jurkat cells (Boatright et al., 2003). The general probe KMB01 showed strong labeling of the endogenous caspases-3, 7, and 9 as well as the exogenously added caspase-8 upon addition of cytochrome c/dATP to the extracts (FIG. 4C). As expected, addition of caspase-8 to un-stimulated extracts led to activation of downstream caspase-3 and 7 and trace amounts of caspase-9. When the caspase-3 selective inhibitor AB06 was added to the extracts in conjunction with cytochrome c/dATP caspases-3 and 7 were selectively inhibited allowing caspases-8 and 9 to be selectively labeled. Similar labeling experiments using high concentrations of the caspase-8 selective probe bAB19 confirmed that it efficiently labeled the exogenous active caspase-8 and to a lesser extent caspase-3 while showing no labeling of caspase-9 even after stimulation with cytochrome c/dATP. The caspase-9 selective probe bAB38 showed labeling of caspase-9 with cross-reactivity towards caspases-3 and 8. Overall these data suggest that the caspase-3 and 8 selective probes have the potential to label endogenous caspase targets and can discriminate between the various intrinsic and extrinsic caspases.

Labeling of Whole Cells with Caspase 8 Specific Compounds bAB06 and bAB13

As a final test of the utility of the probes we examined their ability to label endogenous caspase targets in intact cells induced to undergo apoptosis by either extrinsic (anti-Fas antibody) or intrinsic (etoposide) signals. The biotinylated probes KMB01, bAB06 and bAB13 produced robust labeling of downstream caspases-3 and 7 in anti-Fas and etoposide treated Jurkat cells. Furthermore this labeling was achieved at relatively low concentrations of probe (1 μM) suggesting that they can gain direct access to the cytosol of cells and have high potential for use as imaging agents. Surprisingly, the related caspase-8 specific compound bAB19, which like bAB06 and bAB13 contains two negatively charged residues, did not show labeling of any caspases in the intact cell system. Similarly, KMB01 did not show labeling of caspase-9 even after etoposide treatment. These findings may be in part due to issue of cell permeability of the probe (for bAB19) or the potentially low levels and rapid turnover of active caspase-8 and 9 under these activation conditions. Active site probes equipped with hydrophobic fluorophores are expected to enhance cell permeability.

### Application of Activity Based Probes to Kinetics Studies of Caspase Activation in Apoptotic Proteomes

Caspase cleavage in cell-free apoptotic proteomes has been studied extensively using antibody-based detection methods and exogenous radiolabeled caspases (Liu et al., 1996; Orth et al., 1996; Rodriguez and Lazebnik, 1999; Slee et al., 1999; Srinivasula et al., 1998). However these studies have not been able to directly monitor the activation of specific endogenous caspases. A cell free extract system allows temporal monitoring of both initiator and executioner caspase activity upon stimulation of the intrinsic apoptosis pathway. Thus, our newly developed caspase-3 specific inhibitors coupled with activity based profiling could be used to directly monitor the kinetics of endogenous caspase activation.

We began by monitoring caspase activation in 293 cell extracts over a period of several hours after addition of cytochrome c/dATP. The general probe KMB01 was added to extracts at various time intervals after activation as described in the paragraph below. The same samples were immunoblotted for caspase-7 and 9 protein levels using specific polyclonal antisera. Within the first 5-10 min. of cytochrome c/dATP addition robust labeling of the highly active downstream executioner caspases 3 and 7 (in the 17-22 kDa range) was observed. This activity peaked at 20-30 minutes and remained high throughout the duration of the experiment. In addition, a number of higher molecular weight bands around 35 kDa in size appeared early in the activation pathway. Immunoprecipitation of these labeled proteins identified the p37 and p32 species as forms of caspase-7 and the p35 and p33 as forms of caspase-9 as described in the paragraph below. This was further confirmed by the location of various intermediates of caspase-7 and 9 observed in the western blots of the same samples.

Identification of novel caspase-7 activation intermediate in apoptotic cell extracts was demonstrated in the following experiments: (a.) Cytosolic extracts (293) were induced to undergo intrinsic apoptosis by addition of cytochrome c and dATP for times from 0 to 240 min. At the end of each time point, the general caspase probe KMB01 was added and extracts were incubated for an additional 30 minutes at 37° C. Labeled caspase active sites were visualized by SDS-PAGE analysis followed by blotting for biotin with streptavidin-HRP. The samples were analyzed by western blot using caspase-7 and 9 specific antibodies. The identities of caspases are indicated based on immunoprecipitation experiments in (b) below. Immunoprecipitation was done with FL-C7, which is full-length caspase-7, ΔN-C7, which is, full-length caspase-7 with the 23 N-terminal amino acids removed, p20, which is mature large subunit of caspase-7 with N-terminal peptide removed, and p20+N-C7, which is the mature large subunit of caspase-7 with the 23 residue N-peptide intact. P35-C9 is the predominant auto-processed mature form of caspase-9 large subunit, p33-C9 is an alternatively processed form of the mature large subunit of caspase-9. (b.) Immunoprecipitation of labeled caspases using specific anti-sera. Cytosolic extracts (293) were activated by addition of cytochrome c/dATP for 10 min (+cyt c/dATP) and then labeled for 30 min with the general caspase probe KMB01 or directly labeled with KMB01 without activation (-cyt c/dATP). Caspases were precipitated using specific anti-sera and analyzed by SDS-PAGE followed by blotting for biotin with streptavidin-HRP. I is input labeled extracts P is the immunoprecipitated pellet. (c.) Inhibition of caspase activity by recombinant Bir3 domain. Cytosolic extracts were activated as in (a.) for 5 minutes followed by addition of 1 μM Bir3. KMB01 (20 μM) was added for 30 minutes to label residual caspase active sites as in (a).

Thus we could assign the identity of the p37 band as the full-length caspase-7 with intact N-terminus (FL-C7) and the p32 species as the full-length caspase-7 with loss of the N-terminal peptide (ΔN-C7) (Denault and Salvesen, 2003).

The labeling of a full-length caspase-7 was unexpected as this executioner caspase is thought to be activated in vivo only after removal of the N-peptide by caspase-3 and processing of the zymogen to the large and small subunits by activity of the initiator caspases (Denault and Salvesen, 2003; Yang et al., 1998). However, we find that the full-length form of caspase-7 is capable of binding the active site probe and that the labeling of this species is enhanced by greater than 10-fold upon activation of the intrinsic death pathway (data not shown). This unexpected result suggests that caspase-7 activation involves a catalytically active intermediate that was previously overlooked due to the inability to measure activity of the full-length zymogen in cytosolic extracts.

The caspase-9 species labeled by KMB01 were assigned as the dominant p35 form of caspase-9 that results from auto-processing of the zymogen at Asp315 (p35-C9) and a p33 form of caspase-9 that results from processing of the zymogen at an alternate residue in the linker region between the large and small subunits (p33-C9). In support of this assignment, a 33 kDa (p33) form of caspase-9 has also been observed in the recombinant enzyme as a result of cleavage within the E305/D306/E307 sequence in the linker region (Stennicke et al., 1999). Surprisingly, we did not label the p37 form of caspase-9 that has been postulated to form as the result of a caspase 3-mediated cleavage at Asp330 in the linker region. This processing is thought to produce an active form of caspase-9 that is refractory to inhibition by the Bir3 domain of XIAP (X-linked Inhibitor of Apoptosis Protein) (Srinivasula et al., 2001). We may have failed to label this species in our extracts because it only exists early in the activation process and may be rapidly converted to other forms of caspase-9. However, this possibility seems unlikely as we performed similar experiments using short time points after cytochrome c/dATP addition and still did not observe a p37 form of caspase-9 by western blot (data not shown).

To further confirm our assignment of the labeled species we treated samples at various activation times with purified recombinant Bir3 domain of XIAP. This polypeptide specifically binds to and inhibits caspase-9 but not caspase-3 or 7. Labeling of both the p35 and p33 forms of caspase-9 was blocked by pre-incubation with the Bir3 protein while neither the p37 nor p33 forms of caspase-7 were inhibited by Bir3. These results confirm that the p33 form of caspase-9 retains sensitivity to Bir3 inhibition and does not likely result from processing of the p37 form of caspase-9. We cannot rule out the possibility that p33 caspase-9 is the result of cleavage by some other protease or itself after it has already been labeled by KMB01 or inhibited by Bir3.

### Application of Selective Inhibitors to Kinetics Studies of Caspase Activation in Apoptotic Proteomes

With the identities of all of the primary forms of active caspases in the extract systems assigned, we next examined the effects of inhibition of downstream caspases 3 and 7 on the activation of precursor caspase forms. In particular, we were interested in investigating the possibility that the processing of active full-length caspase-7 is mediated by the mature forms of caspase-3 and 7. To determine the role of each executioner caspase in the processing of upstream intermediates we performed profiling experiments using the general probe KMB01 in 293 extracts in the presence or absence of the caspase-3 and 7 specific inhibitor AB06 and in caspase-3 deficient MCF-7 extracts. The use of MCF-7 cells, which lack active caspase-3 (Janicke et al., 2001) allowed us to separate processing events mediated by caspase-7 from those medicated by caspase-3.

Profiling of caspase activity in 293 extracts that had been treated with AB06 at the same time as cytochrome c/dATP confirmed the selectivity of our inhibitor. Labeling of all mature downstream caspases (in the 17-22 kDa size range) was completely blocked, while labeling of the processed forms of caspase-9 and precursor forms of caspase-7 was unaltered. Interestingly, there was a dramatic change in the kinetics of activation of the full-length caspase-7 intermediate and complete loss of labeling of the ΔN-C7 form that results from removal of the N-peptide. In particular, FL-C7 showed a late and prolonged activation with a peak at 20 minutes that lasted until the end of the assay at 240 minutes. Similar accumulation of FL-C7 was observed in extracts that were treated with AB06 10 minutes after addition of cytochrome c/dATP. Activation of FL-C7 in MCF-7 cell extracts of the same protein concentration was slightly delayed but showed similar rates of active FL-C7 accumulation and disappearance as those observed in un-inhibited 293 extracts. Together, these data suggest that activation of FL-C7 occurs through a process that is dependent on formation of the apoptosome but independent of the activation of mature forms of caspase-3 and 7. This hypothesis is supported by the fact that rates of formation of FL-C7 are relatively similar regardless of the status of mature executioner caspases. Interestingly, ΔN-C7 was not formed in extracts treated with AB06 nor in MCF-7 extracts consistent with the notion that this N-terminal processing is mediated by caspase-3.

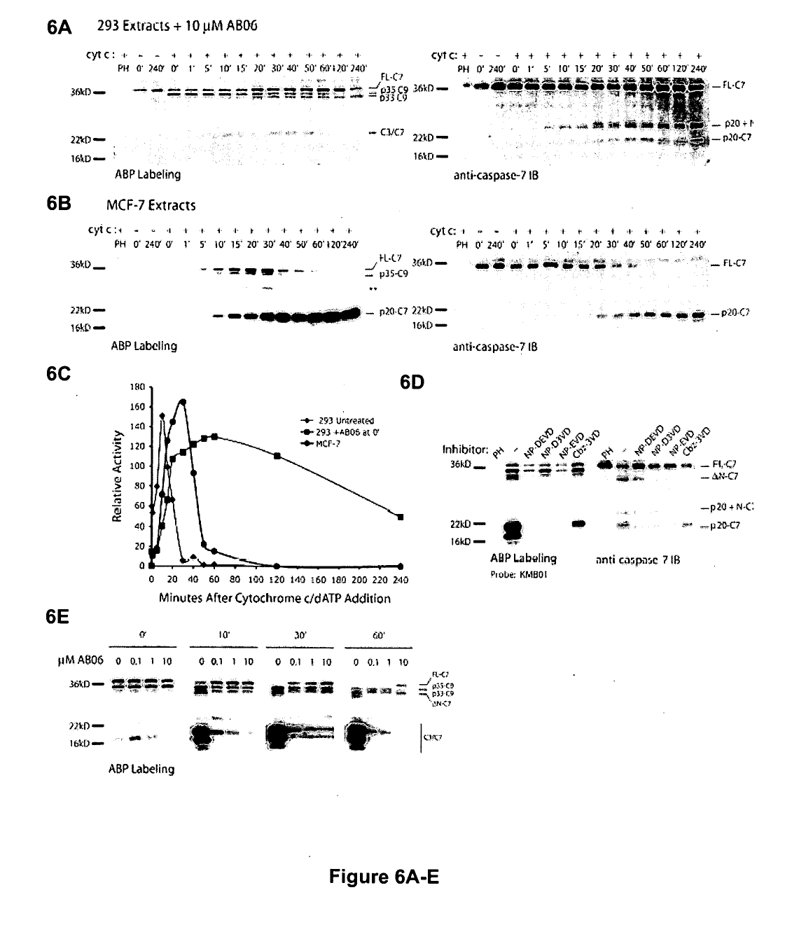

An additional surprising finding from the inhibitor studies was the overall lack of inhibition of the FL-C7 species by AB06 relative to mature p20 forms of caspase-7. This was surprising since the sequence of AB06 (NP-D3VD-AOMK) differs from the KMB01 probe sequence (Bio-EVD-AOMK) only at the P3 residue. We reasoned that this data suggested that FL-C7 has a distinct active site topology that excludes binding of the more bulky P3 NN3 (2pyridylalanine) residue. We therefore wanted to use inhibitors to compare the ability of different P3 elements to bind to full-length and p20 mature forms of caspase-7. Extracts that had been activated by addition of cytochrome c/dATP for 10 min were treated with inhibitors NP-DEVD-AOMK (AB09), NP-D3VD-AOMK (AB06), NP-EVD-AOMK and Cbz-3VD-AOMK for 5 min and then labeled with the general probe KMB01 for 30 min. Samples were analyzed for active site labeling and protein levels of both full-length and p20 forms of caspase-7 were monitored by western blot (FIG. 6D). All four inhibitors efficiently blocked labeling of the mature p20 forms of caspase-7 with the 3VD sequence showing incomplete inhibition. In contrast, FL-C7 was relatively insensitive to inhibition by both inhibitors that contain P3 NN3 and almost totally inhibited by the two inhibitors that contained a P3 Glu residue. Interestingly, this pattern of specificity more closely matched the initiator caspase-9 that also is insensitive to inhibition by P3 NN3 containing probes and inhibitors. We believe these data support the hypothesis that the uncleaved caspase-7 zymogen contains an active site that allows restricted access to substrates.

The accumulation of the FL-C7 species upon treatment of extracts with AB06 suggested that this activation intermediate was likely being processed by downstream executioner caspases rather than the initiator caspase-9. To confirm that our inhibitor AB06 was not reducing caspase 9 activity resulting in slow processing of caspase-7 and accumulation of a partially processed intermediate we treated extracts with a range of AB06 concentrations and monitored caspase-9 inhibition at various time points after cytochrome c/dATP addition. These results confirmed the lack of cross reactivity of AB06 even at concentrations as high as 10 μM for up to 30 minutes. While we did observe some inhibition of caspase-9 by AB06 at the highest concentration and longest time points tested the accumulation of the FL-C7 species was clearly observed at time points and inhibitor concentrations where there was no inhibition of caspase-9. Thus we are confident that the FL-C7 species is an intermediate in the activation pathway that is normally rapidly processed by downstream caspases 3 and 7 and is an inefficient substrate for caspase-9.

### Materials and Methods

### Synthesis Methods for AOMK Inhibitors, Labels and Substrates

All inhibitors and activity based probes were synthesized using solid phase synthesis methods previously reported for P1 Asp-AOMK compounds. All positional scanning peptide libraries were synthesized as reported previously (Greenbaum et al., 2002; Nazif and Bogyo, 2001, hereby incorporated by reference). Briefly, Fmoc-Asp-AOMK loaded resin was elongated by removal of the FMOC protecting group followed by coupling with either a single amino acid for constant positions or an isokinetic mixture of 19 natural amino acids (all natural amino acids minus cysteine with norleucine in place of methionine to prevent oxidation) for mixture positions. Each of the P2-P4 (or P2-P3) positions was scanned with 19 natural amino acids and 40 non-natural amino acids (see FIG. 10) for structures of non-natural amino acids used). All libraries were synthesized on a 50 μmol scale and assayed as crude mixtures after cleavage from the resin. Individual inhibitors and active site probes were synthesized on a 100 μmol scale and purified using a C18 reverse phase HPLC column (Delta-Pak, Waters Corp). Compound identity and purity was assessed by LC-MS analysis using an Agilant HPLC coupled to an API 150 mass spectrometer (Applied Biosystems/SCIEX) equipped with an electrospray interface.

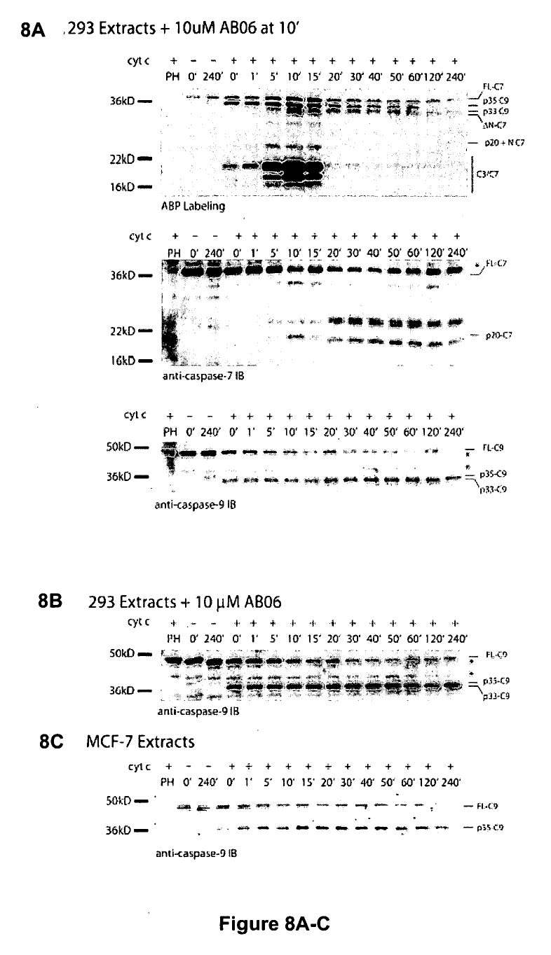

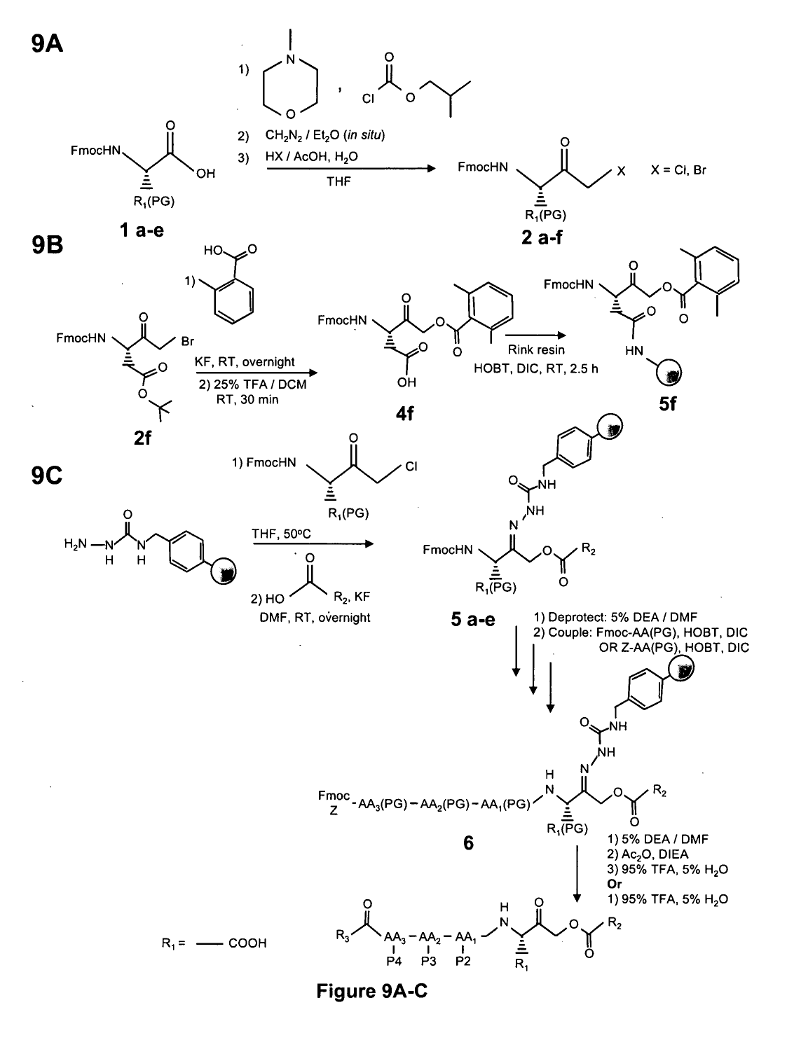

The reaction scheme in FIG. 9 illustrates a solid phase reaction scheme, which may be used for the present AOMK inhibitors. Step (a) shows the synthesis of Fmoc-protected chloromethyl and bromomethyl ketones (2a-f) containing a range of amino acid side chains R1(PG). Step (b) shows solid-phase synthesis of an example of 2a-f, PI asparagine AOMK peptides. The Fmoc protected Asp-AOMK (4f) was synthesized from the corresponding BMK (2f) and was linked directly to a Rink amide resin through its side chain carboxylate (5f). Solid-phase peptide synthesis and resin cleavage methods outlined in step (c) c were used to produce a PI asparagine AOMK shown at the bottom of the reaction scheme, having the structure including AA3-AA2-AA1-, and R1=aspartate (—C—COOH). Step (c) shows solid-phase synthesis of peptide AOMKs using a hydrazine resin. Peptide chloromethyl ketones (2a-e) were linked to the resin through a hydrazone linkage (5a-e) and extended using the indicated optimized solid-phase peptide synthesis method (6). Exemplary compounds in the scheme below are numbered and assigned lowercase letters based on the identity of the PI side chain: a, glycine; b, arginine; c, leucine; d, lysine; e, aspartic acid; f, asparagine. The side chains used will be those given in Tables I-IV above. AcOH, acetic acid; DCM, dichloromethane; DIC, N,N′-diisopropylcarbodiimide; Fmoc, 9-fluorenyl methoxycarbonyl; HOBT, 1-hydroxybenzotriazole; TFA, trifluoroacetic acid; THF, tetrahydrofuran; RT, room temperature; Z, benzyloxycarbonyl. In the scheme on the page below, R2 will be as defined above. R3 will be the same as R1.

The synthesis scheme of the AOMK peptides as shown in FIG. 9 is taken from Kato et al., Unless otherwise noted, all resin and reagents were purchased from commercial suppliers and used without further purification. All solvents used were of HPLC grade. All water-sensitive reactions were performed in anhydrous solvents and under a positive pressure of argon. Reactions were analyzed by thin-layer chromatography on Whatman 0.25 μm silica plates with fluorescent indicator. Flash chromatography was carried out with EMD 230-400 mesh silica gel. Reverse-phase HPLC was conducted on a C₁₈ column using the ÄKTA explorer 100 (Amersham Pharmacia Biotech). LCMS data were acquired using an API 150EX LC/MS system (Applied Biosystems). High-resolution MS analyses were performed by Stanford Proteomics and Integrative Research Facility using a Bruker Autoflex MALDI TOF/TOF mass spectrometer.

The halomethyl ketone precursors (compounds 2a-f above) and their solid support bound derivatives via carbazate linker (5a-e) were synthesized with modification to the procedure as described below. Unless otherwise noted, reactions were conducted in 12-mL polypropylene cartridges (Applied Separations, Allentown, Pa.) with 3-way nylon stopcocks (BioRad Laboratories, Hercules, Calif.). The cartridges were connected to a 20 port vacuum manifold (Waters, Milford, Mass.) that was used to drain solvent and reagents from the cartridge. The resin was gently shaken on a rotating shaker during solid-phase reactions.

General Method for Synthesis of Halomethyl Ketone Derivatives of N-α-Fmoc-Protected Amino Acids (2a-f).

A 0.2 M solution of the corresponding N-α-Fmoc amino acid (1a-e, 5 mmol) in anhydrous THF was stirred in an ice/acetone bath at −10° C. To this solution, N-methylmorpholine (6.25 mmol, 1.25 equiv) and isobutylchloroformate (5.75 mmol, 1.15 equiv) were sequentially added. Immediately after the addition of the latter compound, a white precipitate formed. The reaction mixture was maintained at −10° C. for 25 min. Diazomethane was generated in situ using the procedure described in the Aldrich Technical Bulletin (AL-180). Ethereal diazomethane (16.6-21.4 mmol) was transferred to the stirred solution of the mixed anhydride at 0° C. The reaction mixture was warmed to room temperature over the course of 3 hours. To obtain the corresponding chloromethyl ketones (2a-e), 15 mL of a 1:1 solution of concentrated hydrochloric acid and glacial acetic acid was added dropwise to the reaction mixture at 0° C. Immediately after the evolution of nitrogen gas stopped, the reaction mixture was diluted with ethyl acetate and transferred to a separatory funnel. The reaction mixture was washed sequentially with water, brine solution, and saturated aqueous NaHCO₃. The organic layer was dried over MgSO₄. The solvent was removed under reduced pressure. Alternatively, the bromethyl ketone (2f) was obtained by dropwise addition of 10 mL of a 1:2 solution of hydrogen bromide (30 wt. % solution in acetic acid) and water to the reaction mixture at 0° C. Workup was carried out as described for the chloromethyl ketone synthesis. Chloromethyl ketones 2a (glycine), 2c (leucine), 2d (lysine), 2e (aspartic acid) were obtained as a white solid (quantitative yield) and the bromomethyl ketone 2f (aspartic acid) was obtained as a yellow oil (quantitative yield), and used without any purification. Other amino acid residues were substituted as described above. Crude chloromethyl ketone 2b (arginine) was purified by column chromatography (50-60% ethyl acetate in hexane) to obtain a white solid (3.13 mmol, 62%).

### Synthesis of Carbazate Linker on Aminomethylpolystyrene Resin.

Aminomethylpolystyrene resin (1.1 mmol/g) was dried in vacuo overnight in a 12-mL polypropylene cartridge. The resin was presolvated with DMF for 30 min and another 30 min with CH₂Cl₂. A 1 M solution of N,N′-Carbonyldiimidazole (6 equiv) in CH₂Cl₂ was added to the resin, and the resin was shaken at room temperature for 3 h. The reagent was drained and the resin was washed with CH₂Cl₂ followed by DMF. A 10 M solution of hydrazine (60 equiv) in DMF was added to the resin, and the resin was shaken at room temperature for 1 h. The resin was washed with DMF followed by CH₂Cl₂, dried in vacuo, and stored at −4° C.

Loading of Chloromethyl Ketone Derivatives and Synthesis of 2,6-dimethylbenzoyloxymethyl Ketone Derivatives (5a-e).

A 0.5 M solution of the chloromethyl ketone derivative of the corresponding N-α-Fmoc-L-amino acid (2a-e) in DMF was added to the resin. The cartridge was tightly sealed and shaken at 50° C. for various time periods depending on the chloromethyl ketone. The glycine CMK (2a) was incubated for 10 min; all others (2b-e) were incubated for 3 h. After the reaction the solution was removed, and the resin was washed with DMF. Formation of the AOMK on resin was performed using KF as reported for solution phase synthesis of AOMKs. This method allowed the use of a reduced amount of the carboxylic acid. Specifically a 0.5 M solution of 2,6-dimethylbenzoic acid (5 equiv) and potassium fluoride (10 equiv) in DMF were added to the resin. The resin was shaken at room temperature overnight. After the solution was removed, the resin was washed with DMF followed by CH₂Cl₂, and dried in vacuo. The resin load was estimated by UV absorption of free Fmoc.

Synthesis of 2,6-dimethylbenzoyloxymethyl Ketone Derivative of N-α-Fmoc-L-Aspartic Acid on Rink Resin (5f).

A 0.2 M solution of bromomethyl ketone derivative of N-α-Fmoc-L-aspartic acid-β-tert-butyl ester (2f) in DMF was stirred at 0° C., and potassium fluoride (3 equiv) was added as a solid. After 1 min stirring at 0° C., 2,6-dimethylbenzoic acid (1.2 equiv) was added as a solid, the reaction mixture was warmed to room temperature. After overnight stirring, the reaction mixture was diluted with ethyl acetate, and transferred to a separatory funnel. The reaction mixture was worked up sequentially with water, brine solution, and saturated aqueous NaHCO₃. The organic layer was dried over MgSO₄. The solvent was removed under reduced pressure. The product was purified by flash chromatography (˜17% ethyl acetate in hexane) yielding a yellow oil (98% yield).

A 0.2 M solution of the product 2,6-Dimethylbenzoyloxymethyl ketone derivative of N-α-Fmoc-L-Aspartic Acid-β-tert-butyl ester (3f) was dissolved in 25% v/v TFA/CH₂Cl₂ and allowed to stand for 30 min with occasional shaking. The reaction mixture was diluted with CH₂Cl₂. The cleavage solution was removed by coevaporation with toluene. The product was further dried in vacuo. The crude product (4f; 96% yield) was used without further purification.

Rink resin (0.75 mmol/g) was presolvated by shaking in DMF for 1 h. The Fmoc-protecting group on the resin was removed with 20% piperidine/DMF for 15 min. The resin was washed with DMF followed by CH₂Cl₂. A 0.5 M solution of 2,6-dimethylbenzoyloxymethyl ketone derivative of N-α-Fmoc-L-aspartic acid (4f, 1.25 equiv) and HOBT (1.25 equiv) was added to the resin followed by DIC (1.25 equiv). After shaking for 2.5 h, the resin was washed with DMF, yielding the loaded resin (5f). Resin load was determined by UV absorption of free Fmoc.

### Optimization of Base Deprotection of Peptide AOMKs.

Before solid phase peptide synthesis could be carried out for extended peptides a survey of optimal bases for deprotection of the Fmoc group was performed to identify conditions that allowed Fmoc removal without displacement of the AOMK group. Eighteen aliquots of N-α-Fmoc-L-leucine 2,6-dimethylbenzoyloxymethyl ketone loaded resin (5c, ˜1 mg, ˜3.7×10⁻⁴ mmol) were solvated with DMF for 30 min. DMF solutions of each of the bases were added to each well, and the reactions were shaken for 20 min. The resins were washed with DMF followed by CH₂Cl₂. Acetic anhydride (10 equiv) and DIEA (15 equiv) in 250 μL DMF were added to each well to acylate the deprotected free amine. The reactions were shaken for 15 min and the resin washed with DMF followed by CH₂Cl₂. The reaction block was placed under vacuum for ˜15 min. 200 μL of cleavage cocktail (95% TFA, 5% H₂O) was added to the resin. After 1 h the cleavage mixture were collected, diluted in methanol, and analyzed by direct infusion ion-spray mass spectrometry.

Solid Phase Peptide Synthesis on aminomethylpolystyrene

N-Fmoc-protected 2,6-dimethylbenzoyloxymethyl ketone derivatives linked to aminomethylpolystyrene or Rink resin (5a-f) were presolvated in DMF for 30 min. N-terminal Fmoc group was removed by treatment with a 5% diethylamine solution in DMF for 15 min followed by another 15 min treatment with fresh solution. The resin was washed with DMF followed by CH₂Cl₂. A 0.2 M solution of N-Fmoc-protected amino acid (3 equiv) (Z-protected amino acid for 8, 9 a-c), HOBT (3 equiv) in DMF and DIC (3 equiv) were sequentially added to the resin. The resin was shaken at room temperature for 2 h, and washed with DMF followed by CH₂Cl₂. For each subsequent step of the solid phase peptide synthesis, the same deprotection and coupling reactions were followed. Deprotection and coupling reactions were monitored by the ninhydrin test for primary amine. Capping of the N-terminal amine for the final compound was achieved by shaking the resin with a 0.5 M solution of acetic anhydride (10 equiv) and DIEA (15 equiv) in DMF. After shaking at room temperature for 15 min, the resin was washed with DMF followed by CH₂Cl₂, and dried in vacuo.

General Method of Cleavage from Aminomethylpolystyrene Resin.

The 3-way nylon stopcocks were replaced with TFA-resistant polypropylene needle valve (Waters). A solution of 95% TFA/5% H₂O was added to the resin. After standing at room temperature for 1.5 h, the cleavage mixture was collected, and the resin was washed with fresh cleavage solution. The combined mixture was precipitated in cold ether at −20° C. for 2 h. The precipitated peptide was collected by centrifugation at 3,000 rpm at −10° C. for 15 min. The pellet was dried by positive flow of argon, dissolved in a minimal amount of DMSO. The product was purified on a C₁₈ reverse phase HPLC (Waters, Delta-Pak) using a linear gradient of 0-100% water-acetonitrile. Fractions containing product were pooled, then lyophilized to dryness. The identity of the product was confirmed by mass spectrometry.

The General Method of Cleavage from Rink Resin.

The same procedure as for aminomethylpolystyrene resin was followed except that a solution of 20% TFA/2.5% triisopropylsilane in CH₂Cl₂ was added to the resin, and the reaction time was shortened to 15 min.

### Characterization of Compounds.

All final compounds used for biological studies were purified by HPLC and characterized by high-resolution mass spectrometry (HRMS) using a Bruker Autoflex TOF/TOF mass spectrometer.

### Library Screening

Library screening was carried out using recombinant caspases-3, 8, and 9 in caspase reaction buffer (100 mM Tris, 10 mM DTT, 0.1% CHAPS, 10% sucrose, pH 7.4). Caspases were pre-activated by incubation in caspase reaction buffer for 15 minutes at 37° C. before screening. Caspase-3 (10 nM), caspase-8 (20 nM), and caspase-9 (100 nM) were incubated at 37° C. with inhibitor libraries. Concentrations of inhibitor libraries were selected such that they provided a spectrum of residual activity values ranging from 10% to 80% before normalization. For caspase-3 all libraries were screened at 50 nM final concentrations. For caspase-8 natural and non-natural P2 and P4 libraries were screened at 50 nM while natural and non-natural P3 libraries were screened at 500 nM final concentration. For caspase-9 all libraries were screened at 500 nM final concentration. After a 30-minute incubation with the inhibitor libraries, 100 μM fluorescent substrate (DEVD-AFC for caspase-3, LETD-AFC for caspase-8, and LEHD-AFC for caspase-9, Calbiochem) was added and reactions incubated for 15 minutes. Endpoint fluorescent readings (Abs 495 nm/Emis 515 nm) were measured using a Spectramax M5 plate reader (Molecular Devices). Relative fluorescence values were converted to percentages of residual activity relative to uninhibited controls. Values were internally normalized such that lowest percent residual activity was adjusted to 0% and highest percent residual activity was adjusted to 100%. Residual activity values were compared using hierarchical clustering as described (Greenbaum et al., 2000; Greenbaum et al., 2002; Nazif and Bogyo, 2001).

### Kinetic Compound Screening

Compound screening was completed using the progress curve method as described (Salvesen, 1989). All screening was carried out in caspase reaction buffer. The caspase-9 specific compounds AB38, 40, 41 and 42 were screened in caspase reaction buffer and in caspase buffer with 1M sodium citrate instead of sucrose as described (Pop, 2006) The concentrations of active caspases used is as follows: 5 nM active caspase-3, 5 nM active caspase-7, 10 nM active caspase-8, and 50 nM active caspase-9. To ensure full activation, caspases 3/7, 8 and 9 were preincubated at 37° C. for 5, 10, or 40 minutes respectively before kinetic measurements were made.

### Biotin and Caspase-7 and 9 Immunoblots

All protein samples were quenched in SDS sample buffer and boiled for 5 minutes at 90° C. before SDS-PAGE analysis. Samples were separated on 10-20% Tris-Glycine gradient gels (Novex, Invitrogen) as indicated. Proteins were transferred to nitrocellulose (BioRad) membranes. All biotin and caspase-9 blots were blocked for 1 hour in PBST-5% Milk solution and all caspase-7 blots were blocked in PBST-3% BSA. Biotin blots were subsequently washed for 30 minutes in PBST followed by a 45 min incubation in 1:3500 dilution of Streptavidin-HRP (Sigma) in PBST. Caspase-9 blots were incubated overnight in a 1:3000 dilution of the poly-clonal caspase-9 antibody AR-19B (Burnham Institute for Medical Research) (Stennicke et al., 1999) or a 1:2000 dilution of poly-clonal caspase-7 (Cell Signaling Technologies, cat #9492) in PBST-5% Milk solution or PBST-3% BSA. After 2×30 min washes in PBST, antibody blots were incubated in 1:3000 dilution of secondary anti-rabbit (Santa Cruz) in PBST-5% Milk or PBST-3% BSA for 30 minutes. All blots were washed 3×5 min in PBST and visualized using Supersignal West Pico Chemiluminescent Substrate (Pierce).

### Competition and Direct Labeling of Recombinant Caspases

For all competition and direct labeling experiments recombinant caspases were pre-incubated in caspase reaction buffer for 15 minutes at 37° C. For competition experiments, 100 nM of active site titrated caspase was incubated for 30 minutes at 37° C. with appropriate inhibitor and then residual active sites were labeled with 5 μM KMB01 for an additional 30 minutes. For direct labeling, 100 nM caspase-3, 8, and 9 were incubated together in the presence of appropriate ABPs at the indicated concentrations for 30 min at 37° C.

### Cell Culture

Jurkat and MCF-7 cells were cultured in RPMI 1640 supplemented with 10% fetal calf serum (FCS), 2 mM glutamine, 100 U/ml penicillin, 100 μg/ml streptomycin and maintained in 5% CO₂ at 37° C. 293 cells were cultured as above except DMEM was used in place of RPMI 1640.

### Hypotonic Extract Preparation

Hypotonic 293 and MCF-7 extracts were prepared as described previously (Liu et al., 1996).

### Direct Labeling of Endogenous and Exogenous Caspases in Apoptotic 293 and MCF-7 Extracts

Protein concentration of hypotonic extracts was measured using a standard Bradford Protein Assay (BioRad). 293 extracts were obtained at a total protein concentration of 4.7 μg/μL and MCF-7 extracts were diluted to this concentration. Cytochrome c (100 μM final) and dATP (1 mM final) were added to extracts (73 μg of total protein in a final volume of 20 μL) at time zero and incubation was continued at 37° C. for 10 minutes. Activity based probes (at final concentrations indicated) were added and labeling continued for additional 30 minutes. Samples (13.5 μg of total protein) were analyzed on 10-20% Tris-Glycine gradient gels (Novex Invitrogen). Alternatively, recombinant caspase-8 (100 nM) was added to 293 extracts (as above) in conjunction with cytochrome c/dATP (as above) where appropriate. Extracts were labeled with 10 μM of KMB01, bAB19, and bAB38 10 minutes post activation/caspase-8 addition for 30 minutes at 37° C. Samples (as above) were analyzed as above by SDS-PAGE using 10-20% gradient gels (as above).

### Direct Labeling of Endogenous Caspase Activity in Live Jurkat Cells

Cells (3×10⁶) in media (1 ml) were treated with etoposide (2.5 μg; Calbiochem) or anti-Fas antibody (0.5 μg; clone CH11, Upstate Signaling Solutions) for 15 hours. Cells were then incubated for 2 hours with 10 μM final concentrations of KMB01, bVAD-fmk (Calbiochem), bAB19 or 1 μM final concentrations of bAB06 or bAB13 for two hours. Cells were washed 3× in cold PBS and lysed by boiling in 4×SDS sample buffer for 5 minutes at 90° C. Labeled proteins were analyzed by SDS-PAGE and blotting as described above.

### Intrinsic Apoptosis Assay in Cell Extracts

Hypotonic 293 or MCF-7 extracts (4.7 μg/μl total protein concentration; 73 μg of total protein per time point) were activated by addition of cytochrome c (100 uM final) and dATP (1 mM final) for a range of times from 0-240 minutes as indicated. KMB01 (20 uM final) or vehicle control (DMSO) was added at the end of the indicated activation time and labeling was carried out for an additional 30 minutes at 37° C. Samples were quenched by addition of 4×SDS sample buffer followed by boiling for 5 minutes. A portion (13.5 μg total protein) from each time point was analyzed by SDS-PAGE using 10-20% gradient gels followed by blotting for biotin as described above.

### Intrinsic Apoptosis Assay in Cell Extracts in the Presence of Exogenous Bir3

Hypotonic 293 extracts (73 μg of total protein) were activated as described above and allowed to incubate for the indicated times. Recombinant, purified Bir 3 (1 μM) was added to extracts where appropriate and incubation continued for an additional 5 minutes before addition of KMB01 (20 μM final). Labeling was carried out for an additional 30 minutes and samples (13.5 μg total protein) were analyzed by SDS-PAGE and biotin blotting as described above.

### Quantification of Labeling of FL-C7 in Hypotonic 293 Extracts

The intensity of KMB01 labeling of FL-C7 in timecourse assays was quantified using the publicly available program ImageJ (http://rsb.info.nih.gov/ij/).

### Inhibitor Specificity of FL-C7 in Hypotonic 293 Extracts

Hypotonic 293 extracts (73 μg of total protein) were activated at 37° C. for 5 minutes as described above. NP-EVD-AOMK, NP-DEVD-AOMK (AB09), NP-D3VD-AOMK, or NP-3VD-AOMK (20 μM final) were added to extracts for 5 min before KMB01 (20 μM final) was added and allowed to incubate for 30 minutes at 37° C. Samples (13.5 μg total protein) were analyzed by SDS-PAGE and biotin blotting as described above

### Titration of AB06 in Hypotonic 293 Extracts

Hypotonic 293 extracts were activated as described above and AB06 was added after 5 minutes to the final concentrations indicated. After a 5-minute incubation KMB01 (20 μM final) was added and allowed to incubate for 30 minutes. All reactions were carried out at 37° C.

### Immunoprecipitation

Protein A/G agarose beads (40 μL) were preincubated with 5 μg of the indicated antibody overnight in 300 μL IP Buffer (1×PBS pH 7.4, 0.5% NP-40, 1 mM EDTA) at 4°. Antibodies used were as follows: H-277 caspase-3 poly-clonal (cat #: sc-7148, Santa Cruz), caspase-7 mono-clonal (cat# 556541, BD-Pharmingen), caspase-9 AR-19B (Stennicke et al., 1999). After 3× wash in IP Buffer, beads were re-suspended in 300 μL IP-Buffer and sample was added and allowed to incubate with shaking overnight at 4° C. Beads were washed 3× in IP Buffer followed by 3× in 0.9% NaCl. Beads were boiled in 1× sample buffer for 15 minutes. All supernatant samples were acetone precipitated for 2 hours at −80° C., dried, and resuspended in 1× sample buffer. All samples were subjected to SDS-PAGE followed by biotin blot as described above.

### Fluorogenic Substrates

As noted above, the present invention comprises fluorogenic substrates, which have the specificity of the compounds listed in Tables 1-3, based on the selection of residues listed at P2, P3 and P4. These substrates do not necessarily possess the AOMK structure, but may be exemplified by the structure shown in Formula III given above where the D (aspartate) residue is immediately adjacent an amide-linked coumarin or coumarin derivative. Synthesis of these substrates may proceed by a different route than the AOMK inhibitors. Synthetic methods for various peptide-fluorogenic substrates are known. Exemplary synthetic methods are given in, e.g., U.S. Pat. No. 6,680,178 to Harris, et al., issued Jan. 20, 2004, entitled “Profiling of protease specificity using combinatorial fluorogenic substrate libraries,” hereby incorporated by reference. The patent describes a method of preparing a fluorogenic peptide or a material including a fluorogenic peptide. The method includes: (a) providing a first conjugate comprising a fluorogenic moiety covalently bonded to a solid support, the conjugate having a structure according to a specific formula; (b) contacting the first conjugate with a first protected amino acid moiety (pAA¹) and an activating agent, thereby forming a peptide bond between a carboxyl group of pAA¹ and the aniline nitrogen of the first conjugate; (c) deprotecting the pAA¹, thereby forming a second conjugate having a reactive AA¹ amine moiety; (d) contacting the second conjugate with a second protected amino acid (pAA²) and an activating agent, thereby forming a peptide bond between a carboxyl group of pAA² and the reactive A¹ amine moiety; and (e) deprotecting the pAA², thereby forming a third conjugate having a reactive AA² amine moiety; (f) contacting the third conjugate with a third protected amino acid (pAA³) and an activating agent, thereby forming a peptide bond between a carboxyl group of pAA³ and the reactive AA² amine moiety; and (g) deprotecting the pAA³, thereby forming a fourth conjugate having a reactive AA³ amine moiety. For amino acids that are difficult to couple (Ile, Val, etc), free, unreacted aniline may remain on the support and complicate subsequent synthesis and assay operations. A specialized capping step employing the 3-nitrotriazole active ester of acetic acid in DMF efficiently acylates the remaining aniline. The resulting acetic acid-capped coumarin that may be present in unpurified substrate solutions is generally not a protease substrate. P1-substituted resins that are provided by these methods can be used to prepare any ACC-fluorogenic substrate. The following patent, hereby incorporated by reference, also describes synthetic methods which may be adapted to the preparation of the present fluorogenic substrates, and which further provide description of fluorogenic moieties: U.S. Pat. No. 6,372,895 to Bentsen, et al., issued Apr. 16, 2002, entitled “Fluorogenic compounds.”

The present compounds may also serve as fluorogenic substrates when coupled with coumarin and related compounds, including the labels described above, as discussed in the Summary of the Invention, above. The use of the present peptide-coumarin substrates will be analogous to other coumarin substrates, for example The Caspase-6 Assay Kit, produced by Sigma Aldrich, Inc. This fluorometric assay is based on the hydrolysis of the peptide substrate Acetyl-Val-Glu-Ile-Asp-7-amido-4-methyl coumarin [Ac-VEID-AMC] by caspase 6 that results in the release of the fluorophore 7-amido-4-methyl coumarin [AMC]. The present substrates will be advantageous in that they are specific for the caspases listed in the Tables herein.

Other fluorogenic compounds may be used in place of coumarin or chromene, for example, as disclosed in Monsigny et al., “Assay for proteolytic activity using a new fluorogenic substrate (peptidyl-3-amino-9-ethyl-carbazole); quantitative determination of lipopolysaccharide at the level of one pictogram,” EMBO J. 1982; 1(3): 303-306. Another example is Lee et al., “DEVDase detection in intact apoptotic cells using the cell permeant fluorogenic substrate, (z-DEVD)-2-cresyl violet,” BioTechniques, November 2003, 35:1080-1085, which teaches the use of the fluorophore cresyl violet with the peptide sequence DEVD. Again, the present peptide sequences have been shown to have greater affinity for their target caspase than prior art peptide sequences. As another example of a substrate using the present peptide sequences, one may prepare the present peptides as rhodamine-derivatized dimers, where the fluorophore fluorescence is quenched 90-99%. When a protease cleaves the peptide backbone of this complex, the cyclic structure incorporating the fluorophores is broken and two highly fluorescent substituted peptide fragments are generated. See, Komoriya et al., “Assessment of Caspase Activities in Intact Apoptotic Thymocytes Using Cell-permeable Fluorogenic Caspase Substrates,” The Journal of Experimental Medicine, Jun. 5, 2000, Volume 191, Number 11, 1819-1828.

One may also employ the coumarin analogs in the present fluorogenic synthetic enzyme substrates derived from coumarin derivatives 4-methylumbelliferone (4-MU) or 7-amino-4-methylcoumarin (7-AMC).

### Labeled Compounds

The present compounds may be labeled, e.g., with fluorescent dyes, biotin, labels such as quantum dots, radiolabels, etc. Since the present compounds have amino acid-like side chains, methods used to label peptides may be applied to label the present compounds. Examples are given here in the form of biotin labeled compounds bAB06, bAB13, bAB19, and bAB38.

The compounds may contain a fluorescent molecule, i.e., one that emits electromagnetic radiation, especially of visible light, when stimulated by the absorption of incident radiation. The term includes fluorescein, one of the most popular fluorochromes ever designed, which has enjoyed extensive application in immunofluorescence labeling. This xanthene dye has an absorption maximum at 495 nanometers. A related fluorophore is Oregon Green, a fluorinated derivative of fluorescein. The term further includes bora-diaza-indecene, rhodamines, and cyanine dyes. The term further includes the 5-EDANS (Nucleotide analogs adenosine 5′-triphosphate [g]-1-Naphthalenesulfonic acid-5(2-Aminoethylamide) (ATP[g]-1,5-EDANS) and 8-Azidoadenosine 5′-triphosphate [g]-1-Naphthalenesulfonic acid-5(2-Aminoethylamide) (8N3ATP[g]-1,5-EDANS).

Other suitable labels include “bora-diaza-indecene,” i.e., compounds represented by 4,4-difluoro-4-bora-3a,4a-diaza-s-indacene, known as BODIPY® dyes. Various derivatives of these dyes are known and included in the present definition, e.g., Chen et al., “4,4-Difluoro-4-bora-3a,4a-diaza-s-indacene (BODIPY) dyes modified for extended conjugation and restricted bond rotations,” J Org Chem. 2000 May 19; 65(10):2900-6. These compounds are further defined in reference to the structures set out below under the heading “FLUOROPHORES.” In the exemplified BODIPY TMR-X, R1 in fluorophores=benzyl methoxy; the structure is further shown in Scheme 1. The linker is an amide bond to the lysine side chain chosen as part of the dipeptide starting material. Other suitable labels include “rhodamine,” i.e., a class of dyes based on the rhodamine ring structure. Rhodamines include (among others): Tetramethylrhodamine (TMR): a very common fluorophore for preparing protein conjugates, especially antibody and avidin conjugates; and carboxy tetramethyl-rhodamine (TAMRA), used for oligonucleotide labeling and automated nucleic acid sequencing. Rhodamines are established as natural supplements to fluorescein based fluorophores, which offer longer wavelength emission maxima and thus open opportunities for multicolor labeling or staining. The term is further meant to include “sulfonated rhodamine,” series of fluorophores known as Alexa Fluor dyes.

Also suitable are a family of cyanine dyes, Cy2, Cy3, Cy5, Cy7, and their derivatives, based on the partially saturated indole nitrogen heterocyclic nucleus with two aromatic units being connected via a polyalkene bridge of varying carbon number. These probes exhibit fluorescence excitation and emission profiles that are similar to many of the traditional dyes, such as fluorescein and tetramethylrhodamine, but with enhanced water solubility, photostability, and higher quantum yields. Most of the cyanine dyes are more environmentally stable than their traditional counterparts, rendering their fluorescence emission intensity less sensitive to pH and organic mounting media. In a manner similar to the Alexa Fluors, the excitation wavelengths of the Cy series of synthetic dyes are tuned specifically for use with common laser and arc-discharge sources, and the fluorescence emission can be detected with traditional filter combinations.

The cyanine dyes are readily available as reactive dyes or fluorophores coupled to a wide variety of secondary antibodies, dextrin, streptavidin, and egg-white avidin. The cyanine dyes generally have broader absorption spectral regions than members of the Alexa Fluor family, making them somewhat more versatile in the choice of laser excitation sources for confocal microscopy.

Useful labels include metals, which are bound by chelation to the peptide inhibitors of the present invention. In particular, these include radionuclides having decay properties that are amenable for use as a diagnostic tracer or for deposition of medically useful radiation within cells or tissues. Conjugated coordination complexes of the present caspase inhibitors may be prepared with a radioactive metal (radionuclide). The radioactive nuclide can, for example, be selected from the group consisting of radioactive isotopes of Tc, Ru, In, Ga, Co, Pt, Fe, Os, Ir, W, Re, Cr, Mo, Mn, Ni, Rh, Pd, Nb, Cu and Ta. Exemplary isotopes include Tc-99m, Tc-99, In-111, Ga-67, Ga-68, Cu-64, Ru-97, Cr-51, Co-57, Re-188, I-123, I-125, I-130, I-131, I-133, Sc-47, As-72, Se-72, Y-90, Y-88, Pd-100, Rh-100 m, Sb-119, Ba-128, Hg-197, At-211, Bi-212, Pd-212, Pd-109, Cu-67, Br-75, Br-76, Br-77, C-11, N-13, O-15, F-18, Pb-203, Pb-212, Bi-212, Cu-64, Ru-97, Rh-105, Au-198, and Ag-199 and Re-186.

Further discussion of radioactive labels is found in U.S. Pat. No. 6,589,503 to Piwnica-Worms, issued Jul. 8, 2003, entitled “Membrane-permeant peptide complexes for medical imaging, diagnostics, and pharmaceutical therapy.” Radionuclides that are useful for medical imaging of activated caspases include ¹¹C (t_{1/2} 20.3 min), ¹³N (t_{1/2} 9.97 min), ¹⁸F (t_{1/2} 109.7 min), ⁶⁴Cu (t_{1/2} 12 h), ⁶⁸Ga (t_{1/2} 68 min) for positron emission tomography (PET) and ⁶⁷Ga (t_{1/2} 68 min), ^{99m}Tc (t_{1/2} 6 h), ¹²³I (t_{1/2} 13 h) and ²⁰¹Tl (t_{1/2} 73.5 h) for single photon emission computed tomography (SPECT) (Hom and Katzenellenbogen, Nucl. Med. Biol., 1997, 24:485-498. These metals are coupled to the present peptide like structures. Such coupling is done by preparing conjugated coordination complexes. The peptide metal coordination complexes can be readily prepared by methods known in the art, e.g., as described in the above-referenced patent. For example, a caspase inhibitor peptide to be conjugated to a linker and a metal chelating moiety can be admixed with a salt of the radioactive metal in the presence of a suitable reducing agent, if required, in aqueous media at temperatures from room temperature to reflux temperature, and the end-product coordination complex can be obtained and isolated in high yield at both macro (carrier added, e.g., Tc-99) concentrations and at tracer (no carrier added, e.g., Tc-99m) concentrations (typically less than 10⁻⁶ molar). As is known, when (Tc-99m) pertechnetate (TcO₄) is reduced by a reducing agent, such as stannous chloride, in the presence of chelating ligands such as, but not restricted to, those containing N₂S₂, N₂SO, N₃S and NS₃ moieties, complexes of (TcO)N₂S₂, (TcO)N₂SO, (TcO)N₃S and (TcO)NS₃ are formed (Meegalla et al., J Med. Chem., 1997, 40:9-17. Chelation sites on the present peptide-like structures may be provided as described, e.g., in U.S. Pat. No. 6,323,313 to Tait, et al., issued Nov. 27, 2001, entitled “Annexin derivative with endogenous chelation sites.” This method involves adding certain amino acid residues such as cysteine and glycine to the active sequence. Another method is disclosed in U.S. Pat. No. 5,830,431 to Srinivasan, et al., issued Nov. 3, 1998, entitled “Radiolabeled peptide compositions for site-specific targeting.” This patent discloses a radiolabeled peptide characterized by having its carboxy terminal amino acid in its carboxylic acid form whereby the peptide is coupled to a diagnostic or therapeutic radionuclide by a chelating agent. The chelating agent is capable of covalently binding a selected radionuclide thereto. Suitable chelating agents generally include those which contain a tetradentate ligand with at least one sulfur group available for binding the metal radionuclide such as the known N₃S and N₂S₂ ligands. More particularly, chelating groups that may be used in conjunction with this method and other involving the present compounds include 2,3-bis(mercaptoacetamido)propanoate (U.S. Pat. No. 4,444,690), S-benzoylmercaptoacetylglycylglycylglycine (U.S. Pat. No. 4,861,869), dicyclic dianhydrides such as DTPA and EDTA and derivatives thereof (U.S. Pat. No. 4,479,930), NS chelates containing amino groups to enhance chelation kinetics (U.S. Pat. No. 5,310,536), N₂S₂ chelates as described in U.S. Pat. No. 4,965,392, the N₃S chelates as described in U.S. Pat. No. 5,120,526, and the N.sub.2 S.sub.2 chelates containing cleavable linkers as described in U.S. Pat. No. 5,175,257. The chelating agent is coupled to the peptide-like portion of the present compounds by standard methodology known in the field of the invention and may be added at any location on the peptide provided that the specific active caspase binding activity of the peptide is not adversely affected. Preferably, the chelating group is covalently coupled to the amino terminal amino acid of the peptide. The chelating group may advantageously be attached to the peptide during solid phase peptide synthesis or added by solution phase chemistry after the peptide has been obtained. Preferred chelating groups include DTPA, carboxymethyl DTPA, tetradentate ligands containing a combination of N and S donor atoms or N donor atoms. This method is useful for a variety of radionuclides, including copper.

Also, as described in Thakur et al., “The Role of Radiolabeled Peptide-Nucleic Acid Chimeras and Peptides in Imaging Oncogene Expression,” Indian Journal of Nuclear Medicine, 2004, 19(3):98-114, ⁶⁴Cu may be chelated by methods described for ⁹⁹Tc, by adding ⁶⁴CuCl₂ in 0.1M HCL to purified inhibitor in 0.1M ammonium citrate, pH 5.5, incubation for 20 min at 90° C., quenching with EDTA and purification by size exclusion chromatography.

Labeling with ¹⁸F may be carried out as described in Schottelius et al., “First ¹⁸F-Labeled Tracer Suitable for Routine Clinical Imaging of sst Receptor-Expressing Tumors Using Positron Emission Tomography,” Clinical Cancer Research, Jun. 1, 2004, Vol. 10, 3593-3606. The chemoselective formation of an oxime bond between a radiohalogenated ketone or aldehyde, e.g., 4-[⁸F]-fluorobenzaldehyde, and a peptide functionalized with an aminooxy-functionality is disclosed. This methodology has been applied for radioiodination of antibodies (Kurth M, Pelegrin A, Rose K, et al “Site-specific conjugation of a radioiodinated phenethylamine derivative to a monoclonal antibody results in increased radioactivity localization in tumor,” J Med Chem, 1993, 36: 1255-61) and has been proposed for the radioiodination of small peptides (Thumshirn G, Hersel U, Goodman S L, Kessler H. “Multimeric cyclic RGD peptides as potential tools for tumor targeting: solid-phase peptide synthesis and chemoselective oxime ligation,” Chemistry Eur J, 2003, 9: 2717-2725).

As can be seen from the foregoing, the terminal groups R1 and R2 in Formula I and Formula II may be modified to accommodate a chelation site. R1 may be a peptide chelation site containing about 4 amino acids selected from Cys and Gly. R2 may be COOH, etc.

### Pharmaceutical Compositions

The potential of caspase inhibitors as pharmaceutical agents has been demonstrated with prototype inhibitors in several animal models. Liver diseases like alcoholic liver disease or hepatitis B and C virus infection are associated with accelerated apoptosis. In animal models, the known broad irreversible caspase inhibitor benzyloxycarbonyl-Val-Ala-Asp-fluoromethylketone (z-VAD-fmk) was protective and efficiently blocked death receptor-mediated liver injury (Rodriguez I, Matsuura K, Ody C, Nagata S, and Vassalli P (1996) Systemic injection of a tripeptide inhibits the intracellular activation of CPP32-like proteases in vivo and fully protects mice against Fas-mediated fulminant liver destruction and death. J Exp Med 184: 2067-2072; Kunstle G, Leist M, Uhlig S, Revesz L, Feifel R, MacKenzie A, and Wendel A (1997) ICE-protease inhibitors block murine liver injury and apoptosis caused by CD95 or by TNF-alpha. Immunol Lett 55: 5-10). In arthritis models, repression of proinflammatory cytokine release (IL-1{beta}, IL-18) by blocking its caspase-1-dependent maturation led to efficient reduction of disease severity (Miller B E, Krasney P A, Gauvin D M, Holbrook K B, Koonz D J, Abruzzese R V, Miller R E, Pagani K A, Dolle R E, and Ator M A (1995) Inhibition of mature IL-1 beta production in murine macrophages and a murine model of inflammation by WIN 67694, an inhibitor of IL-1 beta converting enzyme. J Immunol 154: 1331-1338; Ku G, Faust T, Lauffer L L, Livingston D J, and Harding M W (1996) Interleukin-1 beta converting enzyme inhibition blocks progression of type II collagen-induced arthritis in mice. Cytokine 8: 377-386). Myocardial infarction and the resulting death of myocytes was shown to have been ameliorated by z-VAD-fmk and related peptide inhibitors in animal models (Yaoita H, Ogawa K, Maehara K, and Maruyama Y (1998) Attenuation of ischemia/reperfusion injury in rats by a caspase inhibitor. Circulation 97: 276-281.). Also, sepsis that is associated with massive apoptosis of lymphocytes and lethal in approximately 29% of human cases was efficiently reduced in a mouse model by z-VAD-fmk, resulting in increased survival (Hotchkiss R S, Chang K C, Swanson P E, Tinsley K W, Hui J J, Klender P, Xanthoudakis S, Roy S, Black C, Grimm E, et al., (2000) Caspase inhibitors improve survival in sepsis: a critical role of the lymphocyte. Nat Immunol 1: 496-501). In addition, caspase inhibitors reduced neuronal death and infarct size in stroke models (Cheng Y, Deshmukh M, D'Costa A, Demaro J A, Gidday J M, Shah A, Sun Y, Jacquin M F, Johnson E M, and Holtzman D M “Caspase inhibitor affords neuroprotection with delayed administration in a rat model of neonatal hypoxic-ischemic brain injury,” J Clin Investig, 1998, 101: 1992-199). Activation of extrinsic and intrinsic apoptotic pathways has been demonstrated in animal models after spinal cord injury, which was efficiently blocked by z-VAD-fmk, leading to reduced lesion size and improved motor function (Springer J E, Azbill R D, and Knapp P E “Activation of the caspase-3 apoptotic cascade in traumatic spinal cord injury,” Nat Med, 1999, 5: 943-946).

Given the above-described art, one would treat an apotosis-related disease through administration of a compound as described herein, based on present in vivo data, and according to a suitable formulation as described below.

Therapeutic compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal,—or o-cresol, formalin and benzyl alcohol. Standard formulations can either be liquid injectables or solids, which can be taken up in a suitable liquid as a suspension or solution for injection. Thus, in a non-liquid formulation, the excipient can comprise dextrose, human serum albumin, preservatives, etc., to which sterile water or saline can be added prior to administration. One embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Further guidance on formulation and administration of the present compounds may be obtained from U.S. Pat. No. 6,906,037 to Little, I I, et al., issued Jun. 14, 2005, entitled “Therapeutic peptide-based constructs.” As described there, a peptide formulation may be prepared having he BPI protein product may be administered without or in conjunction with known surfactants or other therapeutic agents. A stable pharmaceutical composition containing BPI protein products (e.g., rBPI.sub.23) comprises the BPI protein product at a concentration of 1 mg/ml in citrate buffered saline (5 or 20 mM citrate, 150 mM NaCl, pH 5.0) comprising 0.1% by weight of poloxamer 188 (Pluronic F-68, BASF Wyandotte, Parsippany, N.J.) and 0.002% by weight of polysorbate 80 (Tween 80, ICI Americas Inc., Wilmington, Del.). Another stable pharmaceutical composition containing an active polypeptide at a concentration of 2 mg/ml in 5 mM citrate, 150 mM NaCl, 0.2% poloxamer 188 and 0.002% polysorbate 80. When given parenterally, the product compositions are generally injected in doses ranging from 1 μg/kg to 100 mg/kg per day, preferably at doses ranging from 0.1 mg/kg to 20 mg/kg per day. The treatment may continue by continuous infusion or intermittent injection or infusion, at the same, reduced or increased dose per day for, e.g., 1 to 3 days, and additionally as determined by the treating physician.

### In Vivo Imaging

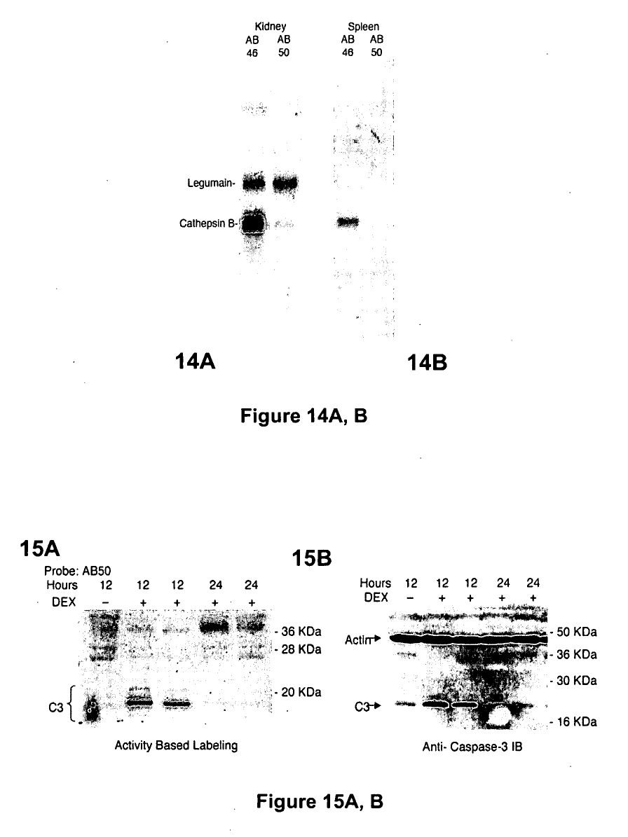

The present selective caspase inhibitors may be used for in vivo imaging. Cy5-fluorescent labeled versions of AB46 and AB50 were injected into normal mice and labeling of cathepsins and legumain in kidney and spleen was monitored (FIG. 14). As anticipated, the probe AB46 showed significant labeling of both legumain and cathepsin B while AB50 only labeled legumain. These results are particularly encouraging because they indicate that the probes are highly selective in vivo (i.e., very low background labeling) and the specificity patterns observed in vitro are retained in vivo.

FIGS. 14 A and B shows in vivo labeling of cathepsin B and legumain by cy5-labeled versions of AB46 and AB50. Normal mice were intravenously injected with the probes and tissues were collected 2 hrs after injection. Tissues were homogenized and total protein samples were analyzed by SDS-PAGE followed by scanning of the gel using a fluorescent scanner.

FIG. 14 shows in vivo labeling of legumain in kidney by AB46 and 50. These compounds may be useful in detecting active legumain in conditions such as atherosclerosis. Further details may be found at US PGPUB 2006/0135410 entitled “Targeted delivery to legumain-expressing cells.”

See also, Papaspyridonos et al., “Novel Candidate Genes in Unstable Areas of Human Atherosclerotic Plaques,” Arteriosclerosis, Thrombosis, and Vascular Biology, 2006; 26:1837.

The following example utilizes dexamethazone-induced apoptosis in the thymus. See, Cristina et al., “Dexamethasone-induced apoptosis of thymocytes: role of glucocorticoid receptor-associated Src kinase and caspase-8 activation,” prepublished online as a Blood First Edition Paper on Aug. 29, 2002; DOI 10.1182/blood-2002-06-1779. This is a particularly simple model that allows one to activate apoptosis specifically in the thymus by injection of normal mice with low doses of dexamethazone. This model allows one to monitor caspase activity in a specific tissue undergoing apoptosis and compare this labeling to tissues that do not contain activated caspases. Also, one can monitor changes in activation of caspases with time after induction of apoptosis.

FIG. 15 shows results from in vivo labeling of caspase-3 in the thymus of dexamethazone treated mice. Wild Type Balb-c mice were injected with dexamethazone (40 mg/kg) by IP injection. After 12 or 24 hrs the general caspase probe AB50 was injected by tail vein and the probe allowed to circulate for 3 hrs, at which time tissues from thymus, lung, liver, and kidney were collected. We removed the thymus, kidney and lung and imaged the whole organs using the IVIS2000 system. The tissues were then analyzed by SDS-PAGE and fluorescent scanning of the gels. For comparison we monitored the levels of cleaved, active caspase-3 using a specific antibody. As shown in FIG. 15, representing results from the thymus, total protein samples from homogenized tissue were analyzed by SDS-PAGE followed by scanning for fluorescence (FIG. 15A) or with a laser flatbed scanner or (FIG. 15B) blotted using antiserum specific for the cleaved form of caspase-3. An antibody against actin was used as a loading control. Bands can be clearly seen at the caspase-3 estimated MW (indicated as “C3”).

AB53 is also caspase-3 specific and is expected to be serum stable and suitable for in vivo use.

These results show that the caspase probes can efficiently and selectively label caspase-3 in the thymus and show little or no labeling in other tissues that are not undergoing apoptosis. The probes may also be used in human breast cancer xenografts treated with chemotherapeutic agents.

The present caspase probes may be used to image tissue undergoing apoptosis as a result of cancer treatment. See Shah et al., “In Vivo Imaging of S-TRAIL-Mediated Tumor Regression and Apoptosis,” Molecular Therapy, June 2005, Vol. 11, No. 926 6. This paper teaches methods for imaging using caspase-3 specific substrates. One may also use probes as disclosed here that are specific for other caspases, for example caspase 7. Caspase 7 is associated with traumatic brain injury. See Zhang et al., “Proteolysis Consistent with Activation of Caspase-7 after Severe Traumatic Brain Injury in Humans,” Journal of Neurotrauma, November 2006, Vol. 23, No. 11: 1583-1590.

### Kits

The present caspase inhibitors may be provided in kits for measuring specific caspase activity in apoptosis and cell signaling. They may also be used to identify other inhibitory drugs. AFC (7-Amino-Trifluoromethyl Coumarin) based substrates yield blue fluorescence upon protease cleavage. A kit is provided which contains a series of AFC-based peptide substrates according to the present description as fluorogenic indicators for assaying caspase protease activities. The kit contains a 96, 384 or other size well plate in which a series of AFC-based caspase substrates are coated with both positive and negative controls. It provides the best solution for profiling caspases or caspase inhibitors. The kit may also contain a cell lysis buffer; assay buffer; AFC (fluorescence reference standard for calibration); and a detailed protocol.

Another kit format utilizes the fact that both caspase-3 and caspase-7 have substrate selectivity for the amino acid sequence Asp-Glu-Val-Asp (DEVD). This kit uses caspase 3-7 selective substrates as the fluorogenic indicator for assaying caspase-3/7 activities. Upon caspase-3/7 cleavage, the substrate generates the coumarin (e.g., AFC fluorophore) which has bright blue fluorescence and can be detected at excitation/emission=380 nm/500 nm. A bi-function assay buffer in this kit is designed to lyze the cells and measure the enzyme activity at the same time. Thus, this kit can measure caspase-3/7 activity in cell culture directly in a 96-well or 384-well plate. The kit may also contain a caspase 8 or 9 substrate.

Another kit format provides active caspases, which cleave the present substrates to release free AFC, which can then be quantified using a microtiter plate reader. Potential inhibitory compounds to be screened can directly be added to the reaction and the level of inhibition of caspases can then be determined. The assays can be performed directly in microtiter plates.

Another kit format comprises an assortment of inhibitors, one selective for caspase 3 and 7, one for caspase 8, one for caspase 9, and one general inhibitor, according to the compound descriptions given above. The compounds are formulated for consistent results and provided with negative controls.

### Modified and Substituted Amino Acids

With regard to the amino acids used in the present compounds, any naturally occurring amino acid may be used. In addition, the amino acids of the peptides of the present invention may also be modified. For example, amino groups may be acylated, alkylated or arylated. Benzyl groups may be halogenated, nitrosylated, alkylated, sulfonated or acylated.

The following residues are preferred for use in the present selective inhibitors: aspartate, valine, glutamate, threonine, proline, leucine, isoleucine, and phenylalanine, as well as specified non-natural side chains 3, 6, 8, 23, 26, 29, 31, 34, 38. Selectivity may be determined by testing with different cysteine proteases as described above. The natural side chains may be further modified. For example, one may use chemically modified amino acids may be incorporated into the present compounds:

### Acetylated

N-acetyl-L-alanine, N-acetyl-L-arginine; N-acetyl-L-asparagine; N-acetyl-L-aspartic acid; N-acetyl-L-cysteine; N-acetyl-L-glutamine; N-acetyl-L-glutamic acid; N-acetylglycine; N-acetyl-L-histidine; N-acetyl-L-isoleucine; N-acetyl-L-leucine; N2-acetyl-L-lysine; N6-acetyl-L-lysine; N-acetyl-L-methionine; N-acetyl-L-phenylalanine; N-acetyl-L-proline; N-acetyl-L-serine;
N-acetyl-L-threonine; N-acetyl-L-tryptophan; N-acetyl-L-tyrosine; N-acetyl-L-valine.

### Amidated

L-alanine amide, L-arginine amide

### Formylated

N-formyl-L-methionine

### Hydroxylated

4-hydroxy-L-proline

### Methylated

N-methyl-L-alanine, N,N,N-trimethyl-L-alanine, omega-N,omega-N-dimethyl-L-arginine, L-beta-methylthioaspartic acid, N5-methyl-L-glutamine, L-glutamic acid 5-methyl ester, 3′-methyl-L-histidine, N6-methyl-L-lysine, N6,N6-dimethyl-L-lysine, N6,N6,N6-trimethyl-L-lysine, N-methyl-L-methionine, N-methyl-L-phenylalanine.

### Phosphorylated

omega-N-phospho-L-arginine, L-aspartic 4-phosphoric anhydride, S-phospho-L-cysteine
1′-phospho-L-histidine, 3′-phospho-L-histidine, O-phospho-L-serine, O-phospho-L-threonine
O4′-phospho-L-tyrosine.

### Other

2′-[3-carboxamido-3-(trimethylammonio)propyl]-L-histidine (diphthamide)
N6-biotinyl-L-lysine
N6-(4-amino-2-hydroxybutyl)-L-lysine (hypusine)

Thus it is to be understood that, for example, “A” refers to naturally occurring Ala, but may also include amidated Ala, as exemplified in the table above. The following amino acids are known to be similar and therefore may be useful in preparing active derivatives of the exemplified compounds. To be “active,” a derivative should have a Ki(app) of at least 500,000, preferably at least 1,000,000. The following substitutions are based on D. Bordo and P. Argos, “Suggestions for ‘Safe’ Residue Substitutions in Site-Directed Mutagensis,” J. Mol. Biol., 1991, 217, 721-729:

A—S, K, P, E

D—N, E

E—D, Q, A

F—Y

I—V, L

L—I,V

P—A

T—S,K

V—I, L

### CONCLUSION

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but which would be understood by workers in the field Such patents or publications are hereby incorporated by reference to the same extent as if each was specifically and individually incorporated by reference, as needed for the purpose of describing and enabling the method or material referred to.

### REFERENCES

1. Amstad, P. A., Yu, G., Johnson, G. L., Lee, B. W., Dhawan, S., and Phelps, D. J. (2001). “Detection of caspase activation in situ by fluorochrome-labeled caspase inhibitors,” Biotechniques 31, 608-610, 612, 614, passim.
2. Backes, B. J., Harris, J. L., Leonetti, F., Craik, C. S., and Ellman, J. A. (2000). “Synthesis of positional-scanning libraries of fluorogenic peptide substrates to define the extended substrate specificity of plasmin and thrombin,” Nat Biotechnol, 2000, 18, 187-193.
3. Berger, A., Sexton, K., Bogyo M., “Commonly used caspase inhibitors designed based on substrate specificity profiles lack selectivity,” Cell Res. (2006): 1-3.
4. Blum, G., Mullins, S. R., Keren, K., Fonovic, M., Jedeszko, C., Rice, M. J., Sloane, B. F., and Bogyo, M. “Dynamic imaging of protease activity with fluorescently quenched activity-based probes,” Nat Chem Biol, 2005, 1, 203-209.
5. Boatright, K. M., Renatus, M., Scott, F. L., Sperandio, S., Shin, H., Pedersen, I. M., Ricci, J. E., Edris, W. A., Sutherlin, D. P., Green, D. R., and Salvesen, G. S. “A unified model for apical caspase activation,” Mol Cell, 2003, 11, 529-541.
6. Boatright, K. M., and Salvesen, G. S. “Mechanisms of caspase activation,” Curr Opin Cell Biol, 2003, 15, 725-731.
7. Chai, J., Wu, Q., Shiozaki, E., Srinivasula, S. M., Alnemri, E. S., and Shi, Y. “Crystal structure of a procaspase-7 zymogen: mechanisms of activation and substrate binding,” Cell, 2001, 107, 399-407.
8. Denault, J. B., and Salvesen, G. S. “Caspases: keys in the ignition of cell death,” Chem Rev, 2002, 102, 4489-4500.
9. Denault, J. B., and Salvesen, G. S. “Human caspase-7 activity and regulation by its N-terminal peptide,” J Biol Chem, 2003, 278, 34042-34050.
10. Deveraux, Q. L., Stennicke, H. R., Salvesen, G. S., and Reed, J. C. “Endogenous inhibitors of caspases,” J Clin Immunol, 1999, 19, 388-398.
11. Eisen, M. B., Spellman, P. T., Brown, P. O., and Botstein, D. “Cluster analysis and display of genome-wide expression patterns,” Proc Natl Acad Sci USA, 1998, 95, 14863-14868.
12. Greenbaum, D., Medzihradszky, K. F., Burlingame, A., and Bogyo, M. “Epoxide electrophiles as activity-dependent cysteine protease profiling and discovery tools,” Chem Biol, 2000, 7, 569-581.
13. Greenbaum, D. C., Arnold, W. D., Lu, F., Hayrapetian, L., Baruch, A., Krumrine, J., Toba, S., Chehade, K., Bromme, D., Kuntz, I. D., and Bogyo, M. “Small molecule affinity fingerprinting. A tool for enzyme family subclassification, target identification, and inhibitor design,” Chem Biol, 2002, 9, 1085-1094.
14. James, K. E., Asgian, J. L., Li, Z. Z., Ekici, 0. D., Rubin, J. R., Mikolajczyk, J., Salvesen, G. S., and Powers, J. C. “Design, synthesis, and evaluation of aza-peptide epoxides as selective and potent inhibitors of caspases-1, -3, -6, and ±8,” J Med Chem, 2004, 47, 1553-1574.
15. Janicke, R. U., Engels, I. H., Dunkern, T., Kaina, B., Schulze-Osthoff, K., and Porter, A. G. “Ionizing radiation but not anticancer drugs causes cell cycle arrest and failure to activate the mitochondrial death pathway in MCF-7 breast carcinoma cells,” Oncogene, 2001, 20, 5043-5053.
16. Joyce, J. A., Baruch, A., Chehade, K., Meyer-Morse, N., Giraudo, E., Tsai, F. Y., Greenbaum, D. C., Hager, J. H., Bogyo, M., and Hanahan, D. “Cathepsin cysteine proteases are effectors of invasive growth and angiogenesis during multistage tumorigenesis,” Cancer Cell, 2004, 5, 443-453.
17. Kato, D., Boatright, K. M., Berger, A. B., Nazif, T., Blum, G., Ryan, C., Chehade, K. A., Salvesen, G. S., and Bogyo, M. “Activity-based probes that target diverse cysteine protease families,” Nat Chem Biol, 2005, 1, 33-38.
18. Liu, X., Kim, C. N., Yang, J., Jemmerson, R., and Wang, X. “Induction of apoptotic program in cell-free extracts: requirement for dATP and cytochrome c,” Cell, 1996, 86, 147-157.
19. Nazif, T., and Bogyo, M. “Global analysis of proteasomal substrate specificity using positional-scanning libraries of covalent inhibitors,” Proc Natl Acad Sci USA, 2001, 98, 2967-2972.
20. Orth, K., O′Rourke, K., Salvesen, G. S., and Dixit, V. M. “Molecular ordering of apoptotic mammalian CED-3/ICE-like proteases,” J Biol Chem, 1996, 271, 20977-20980.
21. Pop, C., Timmer, J., Sperandio, S., Salvesen, G. “The Apoptosome Activates Caspase-9 By Dimerization,” Mol Cell In Press, 2006.
22. Reed, J. C. “Dysregulation of apoptosis in cancer,” Cancer J Sci Am, 1998, 4 Suppl 1, S8-14.
23. Riedl, S. J., Fuentes-Prior, P., Renatus, M., Kairies, N., Krapp, S., Huber, R., Salvesen, G. S., and Bode, W. “Structural basis for the activation of human procaspase-7,” Proc Natl Acad Sci USA, 2001, 98, 14790-14795.
24. Rodriguez, J., and Lazebnik, Y. “Caspase-9 and APAF-1 form an active holoenzyme,” Genes Dev, 1999, 13, 3179-3184.
25. Salvesen, G. S. “Caspases and apoptosis,” Essays Biochem, 2002, 38, 9-19.
26. Salvesen, G. S. a. N., H. “Proteolytic Enzymes: a Practical Approach,” Oxford, IRL Press, 1989.
27. Slee, E. A., Harte, M. T., Kluck, R. M., Wolf, B. B., Casiano, C. A., Newmeyer, D. D., Wang, H. G., Reed, J. C., Nicholson, D. W., Alnemri, E. S., et al., “Ordering the cytochrome c-initiated caspase cascade: hierarchical activation of caspases-2, -3, -6, -7, -8, and -10 in a caspase-9-dependent manner,” J Cell Biol, 1999, 144, 281-292.
28. Srinivasula, S. M., Ahmad, M., Fernandes-Alnemri, T., and Alnemri, E. S. “Autoactivation of procaspase-9 by Apaf-1-mediated oligomerization,” Mol Cell, 1998, 1, 949-957.
29. Srinivasula, S. M., Hegde, R., Saleh, A., Datta, P., Shiozaki, E., Chai, J., Lee, R. A., Robbins, P. D., Fernandes-Alnemri, T., Shi, Y., and Alnemri, E. S. “A conserved XIAP-interaction motif in caspase-9 and Smac/DIABLO regulates caspase activity and apoptosis,” Nature, 2001, 410, 112116.
30. Stennicke, H. R., Deveraux, Q. L., Humke, E. W., Reed, J. C., Dixit, V. M., and Salvesen, G. S. “Caspase-9 can be activated without proteolytic processing,” J Biol Chem, 1999, 274, 8359-8362.
31. Stennicke, H. R., Renatus, M., Meldal, M., and Salvesen, G. S. “Internally quenched fluorescent peptide substrates disclose the subsite preferences of human caspases 1, 3, 6, 7 and 8,” Biochem J, 2000, 350 Pt 2, 563-568.
32. Thornberry, N. A., and Lazebnik, Y. “Caspases: enemies within,” Science, 1998, 281, 1312-1316.
33. Thornberry, N. A., Peterson, E. P., Zhao, J. J., Howard, A. D., Griffin, P. R., and Chapman, K. T. “Inactivation of interleukin-1 beta converting enzyme by peptide (acyloxy)methyl ketones,” Biochemistry, 1994, 33, 3934-3940.
34. Thornberry, N. A., Rano, T. A., Peterson, E. P., Rasper, D. M., Timkey, T., Garcia-Calvo, M., Houtzager, V. M., Nordstrom, P. A., Roy, S., Vaillancourt, J. P., et al., “A combinatorial approach defines specificities of members of the caspase family and granzyme B. Functional relationships established for key mediators of apoptosis,” J Biol Chem, 1997, 272, 17907-17911.
35. Tu, S., McStay, G. P., Boucher, L. M., Mak, T., Beere, H. M., and Green, D. R. “In situ trapping of activated initiator caspases reveals a role for caspase-2 in heat shock-induced apoptosis,” Nat Cell Biol, 2006, 8, 72-77.
36. Yang, X., Stennicke, H. R., Wang, B., Green, D. R., Janicke, R. U., Srinivasan, A., Seth, P., Salvesen, G. S., and Froelich, C. J. “Granzyme B mimics apical caspases. Description of a unified pathway for trans-activation of executioner caspase-3 and -7,” J Biol Chem, 1998, 273, 3427834283.

1. A selective cysteine protease inhibitor represented by the following formula: 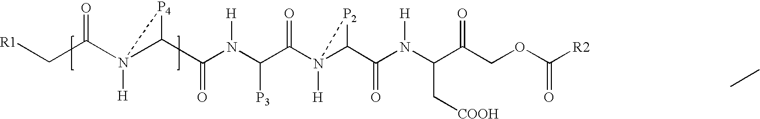
where:
lines P4-N and P2-N indicate bonds which exist only if P4 or P2 are Proline as set forth below;
R1 and R2 are independently selected from the group consisting of H, aminocarbonyl, aryl, substituted aryl (including 2-nitro, 3-hydroxy benzyl), amino, aminocarbonyl, lower alkyl, cycloalkyl, a chelating group for a label or a label,
and P2, P3 and P4 are each a group independently selected from the possible P2, P3 and P4 groups within each caspase as set forth below:
P2 is selected from the group consisting of 8, P, V, T, 23, H, A and 38;
P3 is selected from the group consisting of 16, E, 34, 29, L and F;
P4 is selected from the group consisting of 6, D, 29, 31, L, I and P, with the provisos that
(a) if P4 is 6 or D; P3 is E, 34 or 29 and P2 is V, 8 or P;
(b) if P4 is 29 or 31; P3 is E, and P2 is T or 23;
(c) if P4 is L, I or P; P3 is E, L or F, and P2 is H, A, P or 38,
further provided that P4 in brackets may be omitted (e) if P3 is E or 16 and P2 is 8 or P.
and further providing that A, D, E, H, I, L, P, T, and V are amino acid side chains in standard amino letter code, including side chains which are acetylated, amidated, hydroxylated, methylated, phosphorylated, or oxylated, and where the following substitutions may be made: for A—S, K, P, or E; for D—N, or E; for E—D, Q, or A; for I—V, or L; for L—I, or V; for P—A; for T—S, or K, for 16-17, 26, Y, W, or F, and further providing that the amino acids recited as 6, 8, 16, 17, 23, 26, 29, 31, 34 and 38 have the following structures:

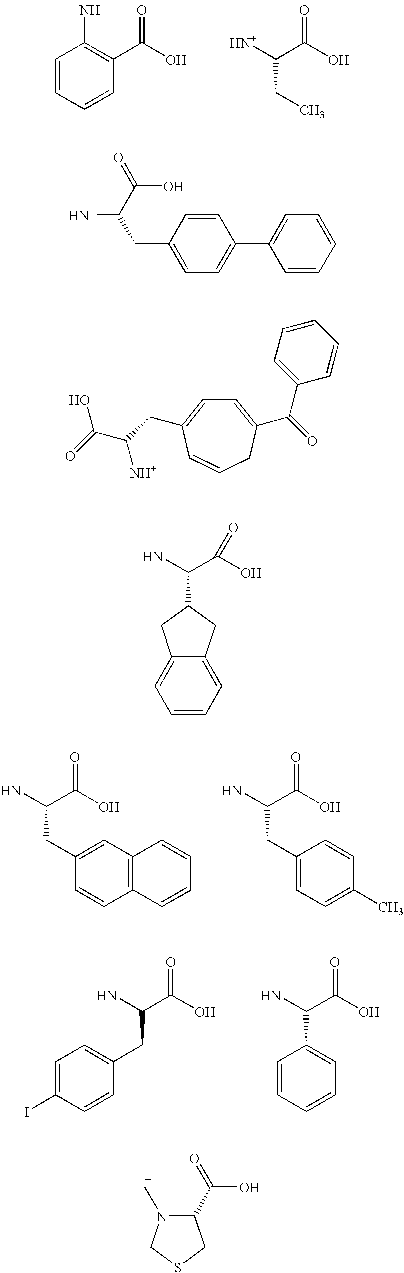

2. A selective cysteine protease inhibitor according to claim 1 having a formula selected from the group (a) through (o) consisting of:

| | P4 | P3 | P2 |
|---|---|---|---|
| (a) | 6 | E | 8 |
| (b) | D | E | P |
| (c) | D | 3 | V |
| (d) | D | 34 | V |
| (e) | D | 29 | V |
| (f) | 29 | E | T |
| (g) | 31 | E | 23 |
| (h) | 31 | E | T |
| (i) | L | E | H |
| (j) | P | L | A |
| (k) | I | F | P |
| (l) | I | L | 38 |
| (m) | omitted | E | 8 |
| (n) | omitted | E | P |
| (o) | omitted | 16 | P |

3. A selective caspase inhibitor according to claim 1 which is selective for one of (a) caspase 3, (b) caspase 7, and (c) caspases 3 and 7.

4. A selective caspase inhibitor according to claim 1 which is selective for caspase 8.

5. A selective caspase inhibitor according to claim 1 which is selective for caspase 9.

6. A selective caspase inhibitor according to claim 1 having a Ki(app) [M⁻¹s⁻¹] greater than 600,000 for a first caspase and a Ki(app) [M⁻¹s⁻¹] less than 150,000 for a second caspase, said first and second caspases being different members of the group consisting of: caspase 3, 7, 8 and 9.

7. A selective caspase inhibitor having a formula of claim 6 wherein P4, P3 and P2, respectively are one of:
none, 16 and P, wherein 16 and P may be substituted as provided in claim 1.

8. A selective caspase inhibitor having a formula of claim 6 wherein P4, P3 and P2, respectively are one of:
D, 3 and V, substituted as provided in claim 1.

9. A selective caspase inhibitor having a formula of claim 6 wherein P4, P3 and P2, respectively are one of:
D, 34 and V, substituted as provided in claim 1.

10. A selective cysteine protease inhibitor having a formula according to claim 1 wherein P4, P3 and P2, respectively are one of: none, E and P.

11. The selective cysteine protease inhibitor of claim 1 wherein the R2 label is selected from a fluorescent dye and a chelated radionuclide.

12. A method of inhibiting a caspase selected from the group consisting of caspase 3, 7, 8 and 9, comprising the step of contacting the caspase with an inhibitory compound according to claim 1.

13. The method of claim 12 wherein the caspase is a human caspase.

14. The method of claim 12 wherein the caspase is inside a cell.

15. The method of claim 12 comprising the step of adding more than one caspase inhibitor.

16. The method of claim 15 wherein caspases 3 and 7 are first inhibited, then caspase 8 or caspase 9 is inhibited.

17. The method of claim 16 wherein the caspase is in an animal.

18. The method of claim 16 wherein the inhibitor is in a pharmaceutical preparation.

19. The method of claim 16 further comprising the step of imaging locations where the inhibitor has inhibited said caspase.

20. A selective, fluorogenic cysteine protease substrate of the formula: 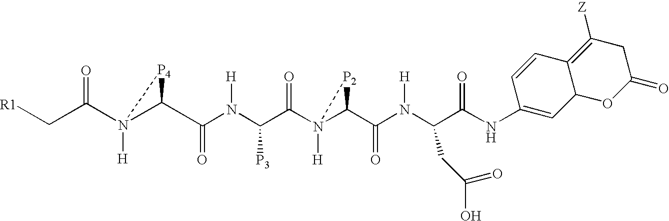
where:
lines N—P2 and N—P4 indicate bonds which exist only if P4 or P2 are P as set forth below;
R1 is H, NH2, aminocarbonyl, aryl, substituted aryl, amino, aminocarbonyl, lower alkyl, or cycloalkyl,
P2, P3 and P4 are each a group independently selected from the possible P2, P3 and P4 groups (a) through (o) listed below,

| | P4 | P3 | P2 |
|---|---|---|---|
| (a) | 6 | E | 8 |
| (b) | D | E | P |
| (c) | D | 3 | V |
| (d) | D | 34 | V |
| (e) | D | 29 | V |
| (f) | 29 | E | T |
| (g) | 31 | E | 23 |
| (h) | 31 | E | T |
| (i) | L | E | H |
| (j) | P | L | A |
| (k) | I | F | P |
| (l) | I | L | 38 |
| (m) | omitted | E | 8 |
| (n) | omitted | E | P |
| (o) | omitted | 16 | P |

the brackets represent a P4 which may be omitted and Z is selected from the group consisting of H, methyl trifluoromethyl and methyl acetamide [—CH2-C(═O)—NH2].

21. A substrate of claim 20 wherein R1 is selected from the group consisting of nitrophenol and biotin-C₄H₈—C(O)—NH—, and benzyl.

22. A method of labeling an active cysteine protease in a cell, comprising the step of administering to the cell a labeled compound having a formula according to claim 1.

23. The method of claim 22 wherein the active cysteine protease is selected from the group consisting of: caspase 3, caspase 7, caspase 8, caspase 9 and legumain.

24. The method of claim 23 wherein the caspase is one of 3, 7, 8 and 9, and the cell is apoptotic.

25. The method of claim 23 wherein the cell is cancerous.

26. The method of claim 22 wherein the cysteine protease is legumain.


SEQUENCE LISTING (11 sequences)

SEQ ID NO: 1 - protein, 4 aa, Artificial Sequence
  Synthetic Construct
DEVD

SEQ ID NO: 2 - protein, 4 aa, Artificial Sequence
  Synthetic Construct
YVAD

SEQ ID NO: 3 - protein, 4 aa, Artificial Sequence
  Synthetic Construct
LETD

SEQ ID NO: 4 - protein, 4 aa, Artificial Sequence
  Synthetic Construct
LEHD

SEQ ID NO: 5 - protein, 4 aa, Artificial Sequence
  Synthetic Construct
PLAD

SEQ ID NO: 6 - protein, 4 aa, Artificial Sequence
  Synthetic Construct
ILAD

SEQ ID NO: 7 - protein, 4 aa, Artificial Sequence
  Synthetic Construct
IFPD

SEQ ID NO: 8 - protein, 4 aa, Artificial Sequence
  Synthetic Construct
DEPD

SEQ ID NO: 9 - protein, 4 aa, Artificial Sequence
  Synthetic Construct
PIED

SEQ ID NO: 10 - protein, 4 aa, Artificial Sequence
  Synthetic Construct
WEHD

SEQ ID NO: 11 - protein, 4 aa, Artificial Sequence
  Synthetic Construct
IETD